(19) United States
(12) Patent Application Publication
(10) Pub. No.: US 2011/0059941 A1
(43) Pub. Date: Mar. 10, 2011

(54) 2-PHENYL SUBSTITUTED IMIDAZOL [4,5B] PYRIDINE/PYRAZINE AND PURINE DERIVATIVES AS GLUCOKINASE MODULATORS

(76) Inventors: Peter William Rodney Caulkett, Cheshire (GB); Darren McKerrecher, Cheshire (GB); Nicholas John Newcombe, Cheshire (GB); Kurt Gordon Pike, Cheshire (GB); Michael James Waring, Cheshire (GB)

(21) Appl. No.: 11/913,219

(22) PCT Filed: May 19, 2006
(86) PCT No.: PCT/GB2006/001842

§ 371 Date: Jul. 31, 2008

(30) Foreign Application Priority Data
May 24, 2005 (GB) 0510503.6
Feb. 22, 2006 (GB) 0603495.3

(51) Int. Cl. A61K 31/437; C07D 471/04; C07D 473/00; A61K 31/52; C07D 487/04; A61K 31/4985; A61K 31/496; A61P 3/10

(52) U.S. Cl. 514/210.18; 546/118; 514/303; 544/264; 514/263.1; 544/350; 514/249; 514/255.05; 544/362; 514/253.04

(57) ABSTRACT

Compounds of Formula (I), wherein R¹-R¹⁰, A and X¹ to X³ are as described in the specification, and their salts and pro-drugs, are activators of glucokinase (GLK) and are thereby useful in the treatment of, for example, type 2 diabetes. Processes for preparing compounds of formula (I) are also described.

The present invention relates to a group of fused imidazo-containing bicyclic compounds which are useful in the treatment or prevention of a disease or medical condition mediated through glucokinase (GLK or GK), leading to a decreased glucose threshold for insulin secretion. In addition the compounds are predicted to lower blood glucose by increasing hepatic glucose uptake. Such compounds may have utility in the treatment of Type 2 diabetes and obesity. The invention also relates to pharmaceutical compositions comprising said compounds and to methods of treatment of diseases to mediated by GLK using said compounds.

In the pancreatic β-cell and liver parenchymal cells the main plasma membrane glucose transporter is GLUT2. Under physiological glucose concentrations the rate at which GLUT2 transports glucose across the membrane is not rate limiting to the overall rate of glucose uptake in these cells. The rate of glucose uptake is limited by the rate of is phosphorylation of glucose to glucose-6-phosphate (G-6-P) which is catalysed by glucokinase (GLK) [1]. GLK has a high (6-10 mM) Km for glucose and is not inhibited by physiological concentrations of G-6-P [1]. GLK expression is limited to a few tissues and cell types, most notably pancreatic β-cells and liver cells (hepatocytes) [1]. In these cells GLK activity is rate limiting for glucose utilisation and therefore regulates the extent of glucose induced insulin secretion and hepatic glycogen synthesis. These processes are critical in the maintenance of whole body glucose homeostasis and both are dysfunctional in diabetes [2].

In one sub-type of diabetes, Maturity-Onset Diabetes of the Young Type 2 (MODY-2), the diabetes is caused by GLK loss of function mutations [3, 4]. Hyperglycaemia in MODY-2 patients results from defective glucose utilisation in both the pancreas and liver [5]. Defective glucose utilisation in the pancreas of MODY-2 patients results in a raised threshold for glucose stimulated insulin secretion. Conversely, rare activating mutations of GLK reduce this threshold resulting in familial hyperinsulinism [6, 6a, 7]. In addition to the reduced GLK activity observed in MODY-2 diabetics, hepatic glucokinase activity is also decreased in type 2 diabetics [8]. Importantly, global or liver selective overexpression of GLK prevents or reverses the development of the diabetic phenotype in both dietary and genetic models of the disease [9-12]. Moreover, acute treatment of type 2 diabetics with fructose improves glucose tolerance through stimulation of hepatic glucose utilisation [13]. This effect is believed to be mediated through a fructose induced increase in cytosolic GLK activity in the hepatocyte by the mechanism described below [13].

Hepatic GLK activity is inhibited through association with GLK regulatory protein (GLKRP). The GLK/GLKRP complex is stabilised by fructose-6-phosphate (F6P) binding to the GLKRP and destabilised by displacement of this sugar phosphate by fructose-1-phosphate (F1P). F1P is generated by fructokinase mediated phosphorylation of dietary fructose. Consequently, GLK/GLKRP complex integrity and hepatic GLK activity is regulated in a nutritionally dependent manner as F6P is dominant in the post-absorptive state whereas F1P predominates in the post-prandial state. In contrast to the hepatocyte, the pancreatic β-cell expresses GLK in the absence of GLKRP. Therefore, β-cell GLK activity is regulated extensively by the availability of its substrate, glucose. Small molecules may activate GLK either directly or through destabilising the GLK/GLKRP complex. The former class of compounds are predicted to stimulate glucose utilisation in both the liver and the pancreas whereas the latter are predicted to act selectively in the liver. However, compounds with either profile are predicted to be of therapeutic benefit in treating Type 2 diabetes as this disease is characterised by defective glucose utilisation in both tissues.

GLK, GLKRP and the K_{ATP} channel are expressed in neurones of the hypothalamus, a region of the brain that is important in the regulation of energy balance and the control of food intake [14-18]. These neurones have been shown to express orectic and anorectic neuropeptides [15, 19, 20] and have been assumed to be the glucose-sensing neurones within the hypothalamus that are either inhibited or excited by changes in ambient glucose concentrations [17, 19, 21, 22]. The ability of these neurones to sense changes in glucose levels is defective in a variety of genetic and experimentally induced models of obesity [23-28]. Intracerebroventricular (icv) infusion of glucose analogues, that are competitive inhibitors of glucokinase, stimulate food intake in lean rats [29, 30]. In contrast, icv infusion of glucose suppresses feeding [31]. Thus, small molecule activators of GLK may decrease food intake and weight gain through central effects on GLK. Therefore, GLK activators may be of therapeutic use in treating eating disorders, including obesity, in addition to diabetes. The hypothalamic effects will be additive or synergistic to the effects of the same compounds acting in the liver and/or pancreas in normalising glucose homeostasis, for the treatment of Type 2 diabetes. Thus the GLK/GLKRP system can be described as a potential “Diabesity” target (of benefit in both Diabetes and Obesity).

GLK is also expressed in specific entero-endocrine cells where it is believed to control the glucose sensitive secretion of the incretin peptides GIP (glucose-dependent insulinotropic polypeptide) and GLP-1 (Glucagon-Like Peptide-1) from gut K-cells and L-cells respectively (32, 33, 34). Therefore, small molecule activators of GLK may have additional beneficial effects on insulin secretion, b-cell function and survival and body weight as a consequence of stimulating GIP and GLP-1 secretion from these entero-endocrine cells.

In WO00/58293 and WO01/44216 (Roche), a series of benzylcarbamoyl compounds are described as glucokinase activators. The mechanism by which such compounds activate GLK is assessed by measuring the direct effect of such compounds in an assay in which GLK activity is linked to NADH production, which in turn is measured optically—see details of the in vitro assay described hereinafter. Compounds of the present invention may activate GLK directly or may activate GLK by inhibiting the interaction of GLKRP with GLK.

Further GLK activators have been described in WO03/095438 (substituted phenylacetamides, Roche), WO03/055482 (carboxamide and sulphonamide derivatives, Novo Nordisk), WO2004/002481 (arylcarbonyl derivatives, Novo Nordisk), and in WO03/080585 (amino-substituted benzoylaminoheterocycles, Banyu).

Our International application Number: WO03/000267 describes a group of benzoyl amino pyridyl carboxylic acids which are activators of the enzyme glucokinase (GLK).

Our International application Number: WO03/015774 describes compounds of the Formula (A):

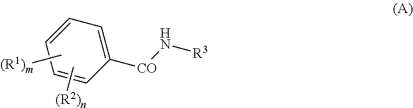

wherein R³ is a substituted heterocycle other than a carboxylic acid substituted pyridyl. One example was included having R³ as a bicyclic heterocycle (benzothiazolyl).

The amide functionality is a common feature of all of the above mentioned compounds.

International application WO 2004/016611 describes the use of imidazopyridine compounds as Inducible T cell kinase inhibitors. Such compounds were known for other uses (see inter alia EP 209707, U.S. Pat. No. 3,985,891 and WO 01/96336), but not as activators of glucokinase. International application WO 2005/63738 (Banyu) describes 2-heteroaryl substituted fused imidazole derivatives (such as 2-heteroaryl substituted benzimidazole compounds) which are glucokinase activators.

We have surprisingly found that fused imidazo-containing bicyclic compounds such as imidazopyridine and imidazopyrazine, not containing central amide functionality are GLK activators. The compounds of the invention have generally good potency for the GLK enzyme, and may have advantageous toxicological and/or physical properties (including, for example, higher aqueous solubility, higher permeability, and/or lower plasma protein binding) which may make them particularly suitable for use in the treatment or prevention of a disease or medical condition mediated through GLK.

Thus, according to the first aspect of the invention there is provided a compound of Formula (I):

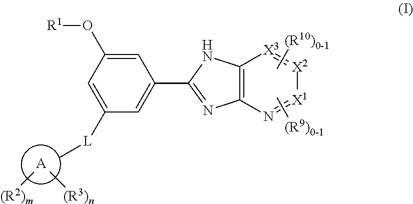

wherein:
Ring A is selected from phenyl and HET-1;
X¹, X² and X³ are each independently CH or N, with the proviso that only one of X¹, X² and X³ may be N;
L is a linker selected from —O— and -(1-3C)alkylO- (wherein the oxygen is directly attached to the benzene ring which is substituted by —OR¹);
R¹ is selected from (1-6C)alkyl, (2-6C)alkenyl, (2-6C)alkynyl, (3-6C)cycloalkyl, (3-6C)cycloalkyl(1-6C)alkyl, aryl(1-6C)alkyl, HET-1 and HET-1-(1-6C)alkyl;
wherein any alkyl, alkenyl, alkynyl, cycloalkyl, aryl or HET-1 group in any definition of R¹ may optionally be substituted on an available carbon atom with a substituent selected from hydroxy, (1-4C)alkoxy, halo, (1-6C)alkylamino, di(1-6C)alkylamino, (CₙH₂ₙ₊₂₋ₐFₐ)—O— (wherein n=1 to 4 and a=1 to 3), (1-6C)alkylsulfonyl, (1-6C)alkylsulfonylamino, (1-6C)alkylsulfonyl-N-[(1-6C)alkyl]amino, (1-6C)alkylaminosulfonyl, di(1-6C)alkylaminosulfonyl, (1-6C)alkylcarbonylamino, (1-6C)alkylcarbonyl-N-[(1-6C)alkyl]amino, (1-6C)alkylaminocarbonyl, di(1-6C)alkylaminocarbonyl, carboxy and cyano; and/or substituted on an available nitrogen atom with a substituent selected from (1-6C)alkylsulfonyl, (1-6C)alkylaminosulfonyl, di(1-6C)alkylaminosulfonyl, (1-6C)alkylaminocarbonyl and di(1-6C)alkylaminocarbonyl;
HET-1 is a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group;
R² is selected from —C(O)NR⁴R⁵, —SO₂NR⁴R⁵, —S(O)ₚR⁴ and HET-2;
HET-2 is a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available nitrogen atom by a substituent selected from R⁶ and/or on an available carbon atom by 1 or 2 substituents independently selected from R⁷;
R³ is selected from halo, fluoromethyl, difluoromethyl, trifluoromethyl, methyl, (1-4C)alkoxy, carboxy and cyano;

R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷), cyano, —NR^{4′}R^{5′} and —C(O)NR⁵R⁵], (3-6C)cycloalkyl (optionally substituted with 1 group selected from R⁷), (2-4C)alkenyl (optionally substituted with 1 group selected from R⁷), (2-4C)alkynyl (optionally substituted with 1 group selected from R⁷), and HET-2;

R⁵ is (independently at each occurrence) selected from hydrogen, (1-4C)alkyl and (3-6C)cycloalkyl;
or R⁴ and R⁵ together with the nitrogen atom to which they are attached may form a heterocyclyl ring system as defined by HET-3;
R⁴′ and R^{5′} are independently selected from hydrogen and (1-4C)alkyl; or
R⁴′ and R^{5′} together with the nitrogen atom to which they are attached may form a 4- to 6-membered saturated ring;
R⁶ is selected from (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
HET-3 is an N-linked, 4 to 7 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸;
R⁸ is selected from —OR⁵, (1-4C)alkyl, (2-4C)alkenyl, (2-4C)alkynyl, trifluoromethyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;
R⁹ is selected from (1-4C)alkyl, halo, cyano, hydroxy(1-4C)alkyl, dihydroxy(2-4C)alkyl, (1-4C)alkoxy(1-4C)alkyl, di(1-4C)alkoxy(2-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl, (1-4C)alkylcarbonylamino, (1-4C)alkylcarbonyl-N-[(1-4C)alkyl]amino, (1-4C)alkylaminocarbonyl, and di(1-4C)alkylaminocarbonyl;
R¹⁰ is selected from methoxy, methyl and halo;
p is (independently at each occurrence) 0, 1 or 2;
m is 0 or 1;
n is 0, 1 or 2;
or a salt or pro-drug thereof;
with the proviso that:
i) neither R⁹ nor R¹⁰ is a substituent on X³;
ii) when R¹ is unsubstituted (1-6C)alkyl then L is —O—.

According to another aspect of the invention, there is provided a compound of formula (I) as hereinbefore defined, wherein

R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷), and —C(O)NR⁵R⁵], (3-6C)cycloalkyl (optionally substituted with 1 group selected from R⁷), and HET-2;
R⁵ is hydrogen or (1-4C)alkyl;
R⁸ is selected from —OR⁵, (1-4C)alkyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;
R⁹ is selected from (1-4C)alkyl, halo, hydroxy(1-4C)alkyl, dihydroxy(2-4C)alkyl, (1-4C)alkoxy(1-4C)alkyl, di(1-4C)alkoxy(2-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl, (1-4C)alkylcarbonylamino, (1-4C)alkylcarbonyl-N-[(1-4C)alkyl]amino, (1-4C)alkylaminocarbonyl, and di(1-4C)alkylaminocarbonyl;
or a salt or pro-drug thereof.
Ring A is selected from phenyl and HET-1;
X¹, X² and X³ are each independently CH or N, with the proviso that only one of X¹, X² and X³ may be N;
L is a linker selected from —O— and -(1-3C)alkylO- (wherein the oxygen is directly attached to the benzene ring which is substituted by —OR¹);
R¹ is selected from (1-6C)alkyl, (2-6C)alkenyl, (2-6C)alkynyl, (3-6C)cycloalkyl, (3-6C)cycloalkyl(1-6C)alkyl, aryl(1-6C)alkyl, HET-1 and HET-1-(1-6C)alkyl;
wherein any alkyl, alkenyl, alkynyl, cycloalkyl, aryl or HET-1 group in any definition of R¹ may optionally be substituted (on an available carbon or nitrogen atom) with a substituent selected from hydroxy, (1-4C)alkoxy, halo, (1-6C)alkylamino, di(1-6C)alkylamino, (CₙH₂ₙ₊₂₋ₐFₐ)—O— (wherein n=1 to 4 and a=1 to 3), (1-6C)alkylsulfonyl, (1-6C)alkylsulfonylamino, (1-6C)alkylsulfonyl-N-[(1-6C)alkyl]amino, (1-6C)alkylaminosulfonyl, di(1-6C)alkylaminosulfonyl, (1-6C)alkylcarbonylamino, (1-6C)alkylcarbonyl-N-[(1-6C)alkyl]amino, (1-6C)alkylaminocarbonyl, di(1-6C)alkylaminocarbonyl, carboxy and cyano;
HET-1 is a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group;
R² is selected from —C(O)NR⁴R⁵, —SO₂NR⁴R⁵, —S(O)ₚR⁴ and HET-2;
HET-2 is a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available nitrogen atom by a substituent selected from R⁶ and/or on an available carbon atom by 1 or 2 substituents independently selected from R⁷;
R³ is selected from halo, fluoromethyl, difluoromethyl, trifluoromethyl, methyl, (1-4C)alkoxy, carboxy and cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷), cyano, —NR^{4′}R^{5′} and —C(O)NR⁵R⁵], (3-6C)cycloalkyl (optionally substituted with 1 group selected from R⁷), (2-4C)alkenyl (optionally substituted with 1 group selected from R⁷), (2-4C)alkynyl (optionally substituted with 1 group selected from R⁷), and HET-2;
R⁵ is (independently at each occurrence) selected from hydrogen, (1-4C)alkyl and (3-6C)cycloalkyl;
or R⁴ and R⁵ together with the nitrogen atom to which they are attached may form a heterocyclyl ring system as defined by HET-3;
R⁴′ and R^{5′} are independently selected from hydrogen and (1-4C)alkyl; or
R⁴′ and R^{5′} together with the nitrogen atom to which they are attached may form a 4- to 6-membered saturated ring;
R⁶ is selected from (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
HET-3 is an N-linked, 4 to 7 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸;
R⁸ is selected from —OR⁵, (1-4C)alkyl, (2-4C)alkenyl, (2-4C)alkynyl, trifluoromethyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;
R⁹ is selected from (1-4C)alkyl, halo, cyano, hydroxy(1-4C)alkyl, dihydroxy(2-4C)alkyl, (1-4C)alkoxy(1-4C)alkyl, di(1-4C)alkoxy(2-4C)alkyl, (1-4C)alkylS(O)ₚ (1-4C)alkyl, amino(1-4C)alkyl, (1-4C)alkylamino(1-4C)alkyl, di(1-4C)alkylamino(1-4C)alkyl, (1-4C)alkylcarbonylamino, (1-4C)alkylcarbonyl-N-[(1-4C)alkyl]amino, (1-4C)alkylaminocarbonyl, and di(1-4C)alkylaminocarbonyl;
R¹⁰ is selected from methoxy, methyl and halo;
p is (independently at each occurrence) 0, 1 or 2;
m is 0 or 1;
n is 0, 1 or 2;
or a salt or pro-drug thereof;
with the proviso that:
i) neither R⁹ nor R¹⁰ is a substituent on X³;
ii) when R¹ is unsubstituted (1-6C)alkyl then L is —O—.

It will be understood that where L is -(1-3C)alkylO-, the alkyl chain may be linear or branched; this definition of L thus encompasses, for example —CH₂—CH₂—O— and —CH₂—CH(Me)-O—.

It will be understood that a (1-6C)alkyl, alkenyl or alkynyl chain in any definition of R¹ may be linear or branched.

It will be understood that when R⁴ is —C(O)NR⁵R⁵, each R⁵ is independently selected from hydrogen and (1-4C)alkyl, and therefore this definition of R⁴ includes (but is not limited to) —CONH₂, —CONHMe, —CONMe₂ and -CONMeEt.

It will be understood that where a compound of the formula (I) contains more than one HET-2 ring, they may be the same or different.

It will be understood that where a compound of the formula (I) contains more than one HET-3 ring, they may be the same or different.

It will be understood that where a compound of the formula (I) contains more than one group R⁴, they may be the same or different.

It will be understood that where a compound of the formula (I) contains more than one group R⁵, they may be the same or different.

It will be understood that where a compound of the formula (I) contains more than one group R⁷, they may be the same or different.

It will be understood that where a compound of the formula (I) contains more than one group R⁸, they may be the same or different.

A similar convention applies for all other groups and substituents on a compound of formula (I) as hereinbefore defined.

It will be understood that R⁹ and R¹⁰ may only be substituents on a ring carbon atom (ie where X═C).

Compounds of Formula (I) may form salts which are within the ambit of the invention. Pharmaceutically acceptable salts are preferred although other salts may be useful in, for example, isolating or purifying compounds.

In another aspect, the invention relates to compounds of formula (I) as hereinabove defined or to a pharmaceutically acceptable salt.

In another aspect, the invention relates to compounds of formula (I) as hereinabove defined or to a pro-drug thereof. Suitable examples of pro-drugs of compounds of formula (I) are in-vivo hydrolysable esters of compounds of formula (I). Therefore in another aspect, the invention relates to compounds of formula (I) as hereinabove defined or to an in-vivo hydrolysable ester thereof.

In this specification the generic term “alkyl” includes both straight-chain and branched-chain alkyl groups. However references to individual alkyl groups such as “propyl” are specific for the straight chain version only and references to individual branched-chain alkyl groups such as t-butyl are specific for the branched chain version only. For example,

“(1-4C)alkyl” includes methyl, ethyl, propyl, isopropyl and t-butyl. An analogous convention applies to other generic terms.

It will be appreciated that, where definitions of heterocylyl groups HET-1-HET-3 encompass heteroaryl rings which may be substituted on nitrogen, such substitution may not result in charged quaternary nitrogen atoms. It will be appreciated that the definitions of HET-1 to HET-3 are not intended to include any O—O, O—S or S—S bonds. It will be appreciated that the definitions of HET-1 to HET-3 are not intended to include unstable structures.

Examples of (1-4C)alkyl include methyl, ethyl, propyl, isopropyl, butyl and tert-butyl; examples of (1-6C)alkyl include (1-4C)alkyl, pentyl and hexyl; examples of (2-4C)alkenyl and (2-6C)alkenyl include vinyl, prop-2-enyl, prop-1-enyl, but-2-enyl and isobutenyl; examples of (2-4C)alkynyl and (2-6C)alkynyl include ehtynyl, prop-1pynyl, prop-2-ynyl, and but-2-ynyl; examples of (3-6C)cycloalkyl include cyclopropyl, cyclobutyl, cyclopentyl and cyclohexyl; examples of (3-6C)cycloalkyl(1-6C)alkyl include cyclopropylmethyl, cyclobutylethyl, cyclopentylpropyl and cyclohexylmethyl; examples of halo include fluoro, chloro, bromo and iodo; examples of hydroxy(1-4C)alkyl include hydroxymethyl, 1-hydroxyethyl, 2-hydroxyethyl, 2-hydroxypropyl, 3-hydroxypropyl, 1-hydroxyisopropyl and 4-hydroxybutyl; examples of dihydroxy(2-4C)alkyl include 1,2-dihydroxyethyl, 1,2-dihydroxypropyl, 1,3-dihydroxypropyl, 2,3-dihydroxypropyl, 1,2-dihydroxybutyl, 1,3-dihydroxybutyl, 2,3-dihydroxybutyl, 3-4-dihydroxybutyl and 2,4-dihydroxybutyl; examples of (1-4C)alkoxy(1-4C)alkyl include methoxymethyl, ethoxymethyl, tert-butoxymethyl, 2-methoxyethyl, 2-ethoxyethyl, methoxypropyl, 2-methoxypropyl and methoxybutyl; examples of di(1-4C)alkoxy(2-4C)alkyl include 1,2-dimethoxyethyl, 1-methoxy-2-ethoxy-ethyl, 1,2-dimethoxypropyl, 1,3-dimethoxypropyl, 2,3-dimethoxypropyl, 1,2-dimethoxybutyl, 2,3-dimethoxybutyl, 2,4-dimethoxybutyl and 3,4-diemthoxybutyl; examples of (1-4C)alkylS(O)p(1-4C)alkyl include methylsulfinylmethyl, ethylsulfinylmethyl, ethylsulfinylethyl, methylsulfinylpropyl, methylsulfinylbutyl, methylsulfonylmethyl, ethylsulfonylmethyl, ethylsulfonylethyl, methylsulfonylpropyl, methylsulfonylbutyl, methylthiomethyl, ethylthiomethyl, ethylthioethyl, methylthiopropyl, and methylthiobutyl; examples of amino(1-4C)alkyl include aminomethyl, aminoethyl, 2-aminopropyl, 3-aminopropyl, 1-aminoisopropyl and 4-aminobutyl; examples of (1-4C)alkylamino(1-4C)alkyl include (N-methyl)aminomethyl, (N-ethyl)aminomethyl, 1-((N-methyl)amino)ethyl, 2-((N-methyl)amino)ethyl, (N-ethyl)aminoethyl, (N-methyl)aminopropyl, and 4-((N-methyl)amino)butyl; examples of di(1-4C)alkylamino(1-4C)alkyl include dimethylaminomethyl, methyl(ethyl)aminomethyl, methyl(ethyl)aminoethyl, (N,N-diethyl)aminoethyl, (N,N-dimethyl)aminopropyl and (N,N-dimethyl)aminobutyl; examples of (1-4C)alkylamino include methylamino, ethylamino, propylamino, isopropylamino, butylamino and tent-butylamino; examples of di(1-4C)alkylamino include dimethylamino, methyl(ethyl)amino, diethylamino, dipropylamino, di-isopropylamino and dibutylamino; examples of —C(O)(1-4C)alkyl include methylcarbonyl, ethylcarbonyl, propylcarbonyl and tert-butyl carbonyl; examples of (1-6C)alkylsulfonyl include methylsulfonyl, ethylsulfonyl, propylsulfonyl, isopropylsulfonyl and tert-butylsulfonyl; examples of (1-6C)alkylsulfonylamino include methylsulfonylamino, ethylsulfonylamino, propylsulfonylamino, isopropylsulfonylamino and tert-butylsulfonylamino; examples of (1-6C)alkylsulfonyl-N-[(1-6C)alkyl]amino include methylsulfonyl-N-(methyl)amino, ethylsulfonyl-N-(methyl)amino, propylsulfonyl-N-(methyl)amino, isopropylsulfonyl-N-(methyl)amino and tert-butylsulfonyl-N-(methyl)amino; examples of (1-6C)alkylaminosulfonyl include methylaminosulfonyl, ethylaminosulfonyl, propylaminosulfonyl, isopropylaminosulfonyl and tert-butylaminosulfonyl; examples of di(1-6C)alkylaminosulfonyl include dimethylaminosulfonyl, diethylaminosulfonyl, methyl(propyl)aminosulfonyl, diisopropylaminosulfonyl and tert-butyl(methyl)aminosulfonyl; examples of (1-6C)alkylaminocarbonyl include (1-4C)alkylaminocarbonyl such as methylaminocarbonyl, ethylaminocarbonyl, propylaminocarbonyl, isopropylaminocarbonyl and tert-butylaminocarbonyl; examples of di(1-6C)alkylaminocarbonyl include di(1-4C)alkylaminocarbonyl such as dimethylaminocarbonyl, diethylaminocarbonyl, methyl(propyl)aminocarbonyl, diisopropylaminocarbonyl and tert-butyl(methyl)aminocarbonyl; examples of (1-6C)alkylcarbonylamino include (1-4C)alkylcarbonylamino such as methylcarbonylamino, ethylcarbonylamino, propylcarbonylamino, isopropylcarbonylamino and tert-butylcarbonylamino; examples of (1-6C)alkylcarbonyl-N-[(1-6C)alkyl]amino include (1-4C)alkylcarbonyl-N-[(1-4C)alkyl]amino methylcarbonyl-N-(methyl)amino, ethylcarbonyl-N-(methyl)amino, propylcarbonyl-N-(methyl)amino, isopropylcarbonyl-N-(methyl)amino and tert-butylcarbonyl-N-(methyl)amino.

Aryl is phenyl or naphthyl, preferably phenyl.

Examples of aryl(1-6C)alkyl include benzyl, phenethyl, phenylpropyl and naphthylmethyl.

Suitable examples of HET-1 as a 5- or 6-membered, C-linked heteroaryl ring as hereinbefore defined, include thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl and triazolyl.

When A is HET-1, further suitable values include thiazolyl, isothiazolyl, thiadiazolyl, pyridyl, pyrazinyl, pyridazinyl, pyrazolyl, imidazolyl, oxazolyl, isoxazolyl and oxadiazolyl.

When A is HET-1, further suitable values include thiazolyl, pyridyl and pyrazinyl.

Suitable examples of HET-1-(1-6C)alkyl include any of the above values for HET-1 in combination with any of the above values for (1-6C)alkyl.

It will be understood that HET-2 can be a saturated, or partially or fully unsaturated ring.

Suitable examples of HET-2 include azetidinyl, furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxazolidinonyl, 2-oxotetrahydrofuranyl, tetrahydrofuranyl, tetrahydropyranyl, 1,1-dioxothiomorpholino, 1,3-dioxolanyl, 1,2,4-triazolyl, 1,2,3-triazolyl, pyranyl, and 4-pyridonyl.

It will be understood that HET-2 may be linked by any appropriate available C or N atom, therefore for example, for HET-2 as “imidazolyl” includes 1-, 2-, 4- and 5-imidazolyl.

Suitable examples of HET-3 as a 4-6 membered saturated or partially unsaturated heterocyclic ring are morpholino, thiomorpholino (and versions thereof wherein the sulfur is oxidised to an SO or S(O)₂ group), piperidinyl, piperazinyl, pyrrolidinyl and azetidinyl. A suitable example of HET-3 as a 7-membered saturated or partially unsaturated heterocyclic ring is homopiperazinyl, homo-morpholino, homo-thiomorpholino (and versions thereof wherein the sulfur is oxidised to an SO or S(O)₂ group) and homo-piperidinyl.

It is to be understood that, insofar as certain of the compounds of Formula (I) defined above may exist in optically active or racemic forms by virtue of one or more asymmetric carbon atoms, the invention includes in its definition any such optically active or racemic form which possesses the property of stimulating GLK directly or inhibiting the GLK/GLKRP interaction. The synthesis of optically active forms may be carried out by standard techniques of organic chemistry well known in the art, for example by synthesis from optically active starting materials or by resolution of a racemic form. It is also to be understood that certain compounds may exist in tautomeric forms and that the invention also relates to any and all tautomeric forms of the compounds of the invention which activate GLK.

It is also to be understood that certain compounds of the formula (I) and salts thereof can exist in solvated as well as unsolvated forms such as, for example, hydrated forms. It is to be understood that the invention encompasses all such solvated forms which activate GLK.

In one aspect, there is provided a compound'of formula (IA) or a salt or pro-drug thereof;

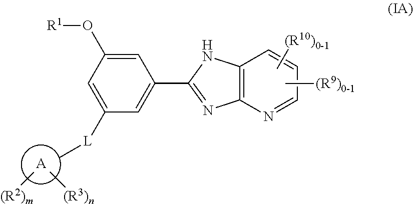

wherein R¹, R², R³, R⁹, R¹⁰, m, n, A and L are as defined for formula (I). It will be understood that the compound of formula (IA) is a compound of formula (I) wherein X¹, X² and X³ are all CH.

In another aspect, there is provided a compound of formula (IB) or a salt or pro-drug thereof;

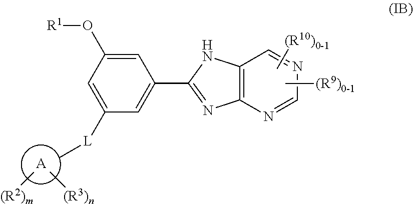

wherein R¹, R², R³, R⁹, R¹⁰, m, n, A and L are as defined for formula (I). It will be understood that the compound of formula (IB) is a compound of formula (I) wherein X¹ and X³ are both CH and X² is N.

In another aspect, there is provided a compound of formula (IC) or a salt or pro-drug thereof;

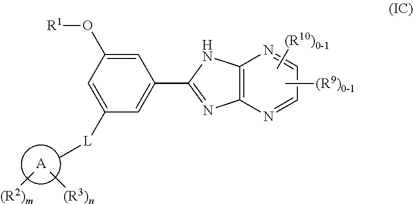

wherein R¹, R², R³, R⁹, R¹⁰, m, n, A and L are as defined for formula (I). It will be understood that the compound of formula (IB) is a compound of formula (I) wherein X¹ and X² are both CH and X³ is N.

It will be appreciated that any aspect or embodiment hereinbefore or after referring to a compound of formula (I) is intended to apply equally to a compound of formula (IA) or a compound of formula (IB) or a compound of formula (IC), even where not explicitly stated.

In one embodiment of the invention are provided compounds of formula (I), in an alternative embodiment are provided pharmaceutically-acceptable salts of compounds of formula (I), (IA), (IB) and (IC), in a further alternative embodiment are provided in-vivo hydrolysable esters of compounds of formula (I), (IA), (IB) and (IC), and in a further alternative embodiment are provided pharmaceutically-acceptable salts of in-vivo hydrolysable esters of compounds of formula (I), (IA), (IB) and (IC).

Preferred values of each variable group are as follows. Such values may be used where appropriate with any of the values, definitions, claims, aspects or embodiments defined hereinbefore or hereinafter. In particular, each may be used as an individual limitation on the broadest definition of formula (I), (IA), (IB) and/or (IC). Further, each of the following values may be used in combination with one or more of the other following values to limit the broadest defintion of formula (I), (IA), (IB) and/or (IC).

(1) R¹ is optionally substituted (1-6C)alkyl, preferably optionally substituted branched (1-6C)alkyl
(2) R¹ is optionally substituted (2-6C)alkenyl
(3) R¹ is optionally substituted (2-6C)alkynyl
(4) R¹ is optionally substituted (3-6C)cycloalkyl
(5) R¹ is optionally substituted (3-6C)cycloalkyl(1-6C)alkyl
(6) R¹ is optionally substituted aryl(1-6C)alkyl
(7) R¹ is optionally substituted HET-1
(8) R¹ is optionally substituted HET-1-(1-6C)alkyl
(9) R¹ is optionally substituted on a carbon atom by hydroxy
(10) R¹ is optionally substituted on a carbon atom by (1-4C)alkoxy
(11) R¹ is optionally substituted on a carbon atom by halo or (CₙH₂ₙ₊₂₋ₐFₐ)—O— (wherein n=1 to 4 and a=1 to 3)
(12) R¹ is optionally substituted on a carbon atom by (1-6C)alkylamino or di(1-6C)alkylamino
(13) R¹ is optionally substituted on a carbon atom by carboxy or cyano
(14) R¹ is optionally substituted on a carbon atom by a substituent selected from (1-6C)alkylsulfonyl, (1-6C)alkylsulfonylamino, (1-6C)alkylsulfonyl-N-[(1-6C)alkyl]amino, (1-6C)alkylaminosulfonyl, di(1-6C)alkylaminosulfonyl, (1-6C)alkylcarbonylamino, (1-6C)alkylcarbonyl-N-[(1-6C)alkyl]amino, (1-6C)aminocarbonyl and di(1-6C)alkylaminocarbonyl
(15) R¹ is hydroxyisopropyl and the configuration is preferably (S), that is R¹ is the group:

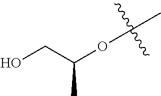

(16) R¹ is methoxyisopropyl and the configuration is preferably (S), that is R¹ is the group:

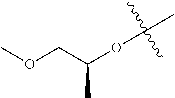

(17) R¹ is isopropyl
(18) R¹ is selected from (1-4C)alkyl (optionally substituted by hydroxy or (1-4C)alkoxy) and HET-1
(19) R¹ is selected from (1-4C)alkyl (optionally substituted by hydroxy or (1-4C)alkoxy) and HET-1 wherein HET-1 is a saturated 5- or 6-membered heterocyclic ring, for example tetrahydrofuranyl or tetrahydropyranyl, particularly tetrahydrofuranyl
(20) R¹ is selected from hydroxyisopropyl, methoxyisopropyl, isopropyl, tert-butoxyisopropyl and tetrahydrofuran-2-yl
(21) Ring A is phenyl

### (22) Ring A is HET-1

(23) Ring A is HET-1 and HET-1 is a fully unsaturated (aromatic) heterocyclic ring
(24) Ring A is phenyl or HET-1 and HET-1 is a fully unsaturated (aromatic) heterocyclic ring
(25) Ring A is HET-1 and HET-1 is selected from pyridyl, pyrimidinyl, pyrazinyl and pyridazinyl
(26) Ring A is HET-1 and HET-1 is selected from pyridyl, pyrimidinyl and pyrazinyl
(27) Ring A is HET-1 and HET-1 is selected from pyridyl and pyrazinyl
(28) Ring A is selected from phenyl, pyridyl, pyrimidinyl and pyrazinyl
(29) Ring A is selected from phenyl, pyridyl and pyrazinyl
(30) Ring A is HET-1 and HET-1 is selected from thiazolyl, pyridyl and pyrazinyl
(31) Ring A is phenyl or HET-1 and HET-1 is a fully unsaturated (aromatic) heterocyclic ring;
and R¹ is selected from (1-4C)alkyl (optionally substituted by hydroxy or (1-4C)alkoxy) and HET-1 wherein HET-1 is a saturated 5- or 6-membered heterocyclic ring, for example tetrahydrofuranyl or tetrahydropyranyl, particularly tetrahydrofuranyl
(32) Ring A is phenyl, pyridyl or pyrazinyl; R¹ is selected from (1-4C)alkyl (optionally substituted by hydroxy or (1-4C)alkoxy) and HET-1 wherein HET-1 is a saturated 5- or 6-membered heterocyclic ring, for example tetrahydrofuranyl or tetrahydropyranyl, particularly tetrahydrofuranyl

### (33) L is —O—

### (34) L is —O—CH₂—

### (35) L is —O—CH₂—CH₂—

### (36) L is —O—CH₂—CH₂—CH₂—

### (37) L is —O—CH(Me)-CH₂—

### (38) L is —O— or —O—CH₂—

(39) Ring A is phenyl or HET-1 and HET-1 is a fully unsaturated (aromatic) heterocyclic ring;
R¹ is selected from (1-4C)alkyl (optionally substituted by hydroxy or (1-4C)alkoxy) and HET-1 wherein HET-1 is a saturated 5- or 6-membered heterocyclic ring, for example tetrahydrofuranyl or tetrahydropyranyl, particularly tetrahydrofuranyl; and L is —O— or —O—CH₂—
(40) Ring A is phenyl, pyridyl or pyrazinyl; R¹ is selected from (1-4C)alkyl (optionally substituted by hydroxy or (1-4C)alkoxy) and HET-1 wherein HET-1 is a saturated 5- or 6-membered heterocyclic ring, for example tetrahydrofuranyl or tetrahydropyranyl, particularly tetrahydrofuranyl; and L is —O— or —O—CH₂—
(41) HET-1 is a 4-membered heterocyclyl ring
(42) HET-1 is a 5- or 6-membered heterocyclyl ring
(43) HET-1 is a 5-membered heterocyclyl ring
(44) HET-1 is a 6-membered heterocyclyl ring

### (45) HET-1 is N-linked

### (46) HET-1 is C-linked

### (47) R² is —C(O)NR⁴R⁵

(48) R² is —C(O)NR⁴R⁵, R⁴ and R⁵ together with the nitrogen atom to which they are attached form a heterocyclyl ring system as defined by HET-3
(49) R² is —C(O)NR⁴R⁵, R⁴ and R⁵ together with the nitrogen atom to which they are attached form a heterocyclyl ring system as defined by HET-3,
selected from morpholino, thiomorpholino (and versions thereof wherein the sulfur is oxidised to an SO or S(O)₂ group), piperidinyl, piperazinyl, pyrrolidinyl and azetidinyl

### (50) R² is —SO₂NR⁴R⁵

### (51) R² is —S(O)ₚR⁴

### (52) R² is HET-2

(53) HET-2 is a 4-membered heterocyclyl ring
(54) HET-2 is a 5- or 6-membered heterocyclyl ring
(55) HET-2 is a 5-membered heterocyclyl ring
(56) HET-2 is a 6-membered heterocyclyl ring

### (57) HET-2 is N-linked

### (58) HET-2 is C-linked

(59) HET-2 is unsubstituted
(60) HET-2 is substituted on a carbon atom with 1 substituent selected from R⁷
(61) HET-2 is substituted on a nitrogen atom with 1 substituent selected from R⁶
(62) R² is HET-2 and HET-2 is a 5- or 6-membered ring, selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, 1,2,4-triazolyl, 1,2,3-triazolyl and pyranyl
(63) R² is HET-2 and HET-2 is a 5- or 6-membered ring, selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl and pyranyl
(64) R² is HET-2 and HET-2 is selected from furyl, thienyl, thiazolyl, isothiazolyl, thiadiazolyl, pyrazolyl, imidazolyl, oxazolyl, isoxazolyl and oxadiazolyl
(65) R² is HET-2 and HET-2 is oxadiazolyl
(66) R³ is selected from halo, (1-4C)alkoxy (such as methoxy) and methyl
(67) R³ is selected from fluoromethyl, difluoromethyl and trifluoromethyl
(68) R³ is selected from carboxy and cyano
(69) R³ is selected from halo, (1-4C)alkoxy (such as methoxy), carboxy and cyano
(70) R⁴ is hydrogen
(71) R⁴ is optionally substituted (1-4C)alkyl
(72) R⁴ is (1-4C)alkyl substituted by HET-2
(73) R⁴ is (1-4C)alkyl substituted by HET-2 and HET-2 is selected from azetidinyl, furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxazolidinonyl, 2-oxotetrahydrofuranyl, tetrahydrofuranyl, tetrahydropyranyl, 1,1-dioxothiomorpholino, 1,3-dioxolanyl, 1,2,4-triazolyl, 1,2,3-triazolyl, pyranyl, and 4-pyridonyl
(74) R⁴ is (1-4C)alkyl substituted by HET-2 and HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxotetrahydrofuranyl, tetrahydrofuranyl, tetrahydropyranyl, 1,1-dioxothiomorpholino, 1,3-dioxolanyl, pyranyl
(75) R⁴ is (1-4C)alkyl substituted by HET-2 and HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, 2,5-dioxopyrrolidinyl, tetrahydrothienyl, 1-oxotetrahydrothienyl, 1,1-dioxotetrahydrothienyl, 2-oxotetrahydrofuranyl, tetrahydrofuranyl, tetrahydropyranyl, 1,1-dioxothiomorpholino
(76) R⁴ is (1-4C)alkyl substituted by HET-2 and HET-2 is selected from thienyl and pyrrolidinyl
(77) R⁴ is (1-4C)alkyl substituted with —OR⁵
(78) R⁴ is (1-4C)alkyl substituted with —SO₂R⁵
(79) R⁴ is (1-4C)alkyl substituted with (3-6C)cycloalkyl (optionally substituted with 1 group selected from R⁷)
(80) R⁴ is (1-4C)alkyl substituted with —C(O)NR⁵R⁵
(81) R⁴ is (1-4C)alkyl substituted with —NR^{4′}R^{5′}
(82) R⁴ is (1-4C)alkyl substituted with —NR^{4′}R^{5′} and R^{4′} and R^{5′} are each independently hydrogen or (1-4C)alkyl, particularly hydrogen or methyl
(83) R⁴ is (1-4C)alkyl substituted with cyano

### (84) R⁴ is (2-4C)alkenyl

### (85) R⁴ is (2-4C)alkynyl

(86) R⁴ is (3-6C)cycloalkyl (optionally substituted with 1 group selected from R⁷)

### (87) R⁴ is HET-2

(88) R⁴ is HET-2 and HET-2 is selected from azetidinyl, furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxazolidinonyl, 2-oxotetrahydrofuranyl, tetrahydrofuranyl, tetrahydropyranyl, 1,1-dioxothiomorpholino, 1,3-dioxolanyl, 1,2,4-triazolyl, 1,2,3-triazolyl, pyranyl, and 4-pyridonyl
(89) R⁴ is HET-2 and HET-2 is selected from azetidinyl, morpholino, morpholinyl, piperidinyl, piperazinyl, thiomorpholino, thiomorpholinyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxazolidinonyl, 2-oxotetrahydrofuranyl, tetrahydrofuranyl, tetrahydropyranyl, 1,1-dioxothiomorpholino, 1,3-dioxolanyl, 1,2,4-triazolyl, 1,2,3-triazolyl, pyranyl, and 4-pyridonyl
(90) R⁴ is HET-2 and HET-2 is selected from piperidinyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxotetrahydrofuranyl, tetrahydrofuranyl, tetrahydropyranyl, 1,1-dioxothiomorpholino and 1,3-dioxolanyl
(91) R⁴ is HET-2 and HET-2 is selected from tetrahydrothienyl, 1-oxotetrahydrothienyl, 1,1-dioxotetrahydrothienyl, 2-oxotetrahydrofuranyl, tetrahydrofuranyl and tetrahydropyranyl
(92) R⁴ is HET-2 and HET-2 is selected from tetrahydrothienyl, 1-oxotetrahydrothienyl, 1,1-dioxotetrahydrothienyl and tetrahydropyranyl
(93) R⁵ is hydrogen

### (94) R⁵ is (1-4C)alkyl

(95) R⁵ is hydrogen or (1-4C)alkyl

### (96) R⁵ is (3-6C)cycloalkyl

(97) R⁴ and R⁵ together with the nitrogen atom to which they are attached form a heterocyclyl ring system as defined by HET-3
(98) R⁴ and R⁵ together with the nitrogen atom to which they are attached form a heterocyclyl ring system as defined by HET-3, selected from morpholino, thiomorpholino (and versions thereof wherein the sulfur is oxidised to an SO or S(O)₂ group), piperidinyl, piperazinyl, pyrrolidinyl, azetidinyl, homopiperazinyl, homo-morpholino, homo-thiomorpholino (and versions thereof wherein the sulfur is oxidised to an SO or S(O)₂ group) and homo-piperidinyl
(99) R⁴ and R⁵ together with the nitrogen atom to which they are attached form a heterocyclyl ring system as defined by HET-3, selected from morpholino, thiomorpholino (and versions thereof wherein the sulfur is oxidised to an SO or S(O)₂ group), piperidinyl, piperazinyl, pyrrolidinyl and azetidinyl
(100) R⁴ and R⁵ together with the nitrogen atom to which they are attached form a heterocyclyl ring system as defined by HET-3, selected from piperidinyl, piperazinyl, pyrrolidinyl and azetidinyl
(101) R⁶ is selected from (1-4C)alkyl, (1-4C)alkoxy(1-4C)alkyl and hydroxy(1-4C)alkyl
(102) R⁶ is selected from —C(O)(1-4C)alkyl, —C(O)NR⁴R⁵, and —S(O)pR⁵
(103) R⁷ is selected from (1-4C)alkyl, (1-4C)alkoxy(1-4C)alkyl and hydroxy(1-4C)alkyl
(104) R⁷ is selected from —OR⁵, —C(O)(1-4C)alkyl, —C(O)NR⁴R⁵, and —S(O)pR⁵
(105) R⁷ is selected from —OR⁵ (wherein R⁵ is hydrogen or (1-4C)alkyl) and hydroxy(1-4C)alkyl
(106) HET-3 is 4-membered ring
(107) HET-3 is a 5-membered ring
(108) HET-3 is a 6-membered ring
(109) HET-3 is a 7-membered ring
(110) HET-3 is unsubstituted
(111) HET-3 is substituted with 1 substituent R⁸
(112) R⁸ is selected from —OR⁵, (1-4C)alkyl, (1-4C)alkoxy(1-4C)alkyl and hydroxy(1-4C)alkyl
(113) R⁸ is selected from —C(O)(1-4C)alkyl, —C(O)NR⁴R⁵, (1-4C)alkylamino and di(1-4C)alkylamino
(114) R⁸ is selected from HET-3 (wherein said ring is unsubstituted)

### (115) R⁸ is —S(O)pR⁵

(116) R⁸ is selected from methoxy and methyl
(117) R⁸ is selected from —OR⁵, (1-4C)alkyl, (2-4C)alkenyl, trifluoromethyl, —C(O)NR⁴R⁵ and hydroxy(1-4C)alkyl
(118) R⁸ is selected from hydroxy, methoxy, (1-4C)alkyl, allyl, trifluoromethyl, methylaminocarbonyl, dimethylaminocarbonyl and hydroxy(1-4C)alkyl (such as hydroxyethyl)
(119) when R⁸ is a substituent on nitrogen, it is particularly selected from (1-4C)alkyl, allyl, methylaminocarbonyl, dimethylaminocarbonyl and hydroxy(1-4C)alkyl (such as hydroxyethyl)
(120) R⁹ is selected from (1-4C)alkyl and halo
(121) R⁹ is selected from hydroxy(1-4C)alkyl, dihydroxy(2-4C)alkyl, (1-4C)alkoxy(1-4C)alkyl and di(1-4C)alkoxy(2-4C)alkyl
(122) R⁹ is selected from (1-4C)alkylS(O)p(1-4C)alkyl
(123) R⁹ is selected from amino(1-4C)alkyl, (1-4C)alkylamino(1-4C)alkyl and di(1-4C)alkylamino(1-4C)alkyl
(124) R⁹ is selected from (1-4C)alkylcarbonylamino, (1-4C)alkylcarbonyl-N-[(1-4C)alkyl]amino, di(1-4C)alkylaminocarbonyl and (1-4C)alkylaminocarbonyl
(125) R⁹ is selected from halo, (1-4C)alkyl and cyano
(126) R⁹ is selected from chloro, fluoro, bromo, methyl and cyano
(127) R⁹ is selected from halo and cyano
(128) R⁹ is selected from chloro, fluoro, bromo and cyano
(129) R⁹ is selected from chloro, fluoro and cyano
(130) R¹⁰ is methoxy
(131) R¹⁰ is methyl
(132) R¹⁰ is halo
(133) R¹⁰ is methoxy, methyl or halo
(134) R¹⁰ is methoxy or halo
(135) one of R⁹ or R¹⁰ is halo and the other is absent
(136) one of R⁹ or R¹⁰ is fluoro and the other is absent
(137) X² is C—R⁹, where R⁹ is fluoro, and R¹⁰ is absent
(138) in a compound of formula (IA), X² is C—R⁹, where R⁹ is fluoro, and R¹⁰ is absent
(139) both R⁹ and R¹⁰ are absent
(140) m is 0
(141) m is 1
(142) n is 0
(143) n is 1
(144) n is 2

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —C(O)NR⁴R⁵;

R³ is halo, methoxy or cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;
R⁵ is hydrogen or (1-4C)alkyl;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁹ is halo, methyl or methoxy;
R¹⁰ is absent;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —C(O)NR⁴R⁵;

R³ is halo, methoxy or cyano;
R⁴ is selected from (1-4C)alkyl;
R⁵ is hydrogen or (1-4C)alkyl;
R⁹ is halo, methyl or methoxy;
R¹⁰ is absent;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —C(O)NR⁴R⁵;

R³ is halo, methoxy or cyano;
R⁴ and R⁵ together with the nitrogen atom to which they are attached may form a heterocyclyl ring system as defined by HET-3;
HET-3 is an azetidine, pyrrolidine or piperidine ring, and is optionally substituted by methoxy, hydroxy or methyl;
R⁹ is halo, methyl or methoxy;
R¹⁰ is absent;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —SO₂NR⁴R⁵;

R³ is halo, methoxy or cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;
R⁵ is hydrogen or (1-4C)alkyl;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁹ is halo, methyl or methoxy;
R¹⁰ is absent;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —SO₂NR⁴R⁵;

R³ is halo, methoxy or cyano;
R⁴ is selected from (1-4C)alkyl;
R⁵ is hydrogen or (1-4C)alkyl;
R⁹ is halo, methyl or methoxy;
R¹⁰ is absent;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —SO₂NR⁴R⁵;

R³ is halo, methoxy or cyano;
R⁴ and R⁵ together with the nitrogen atom to which they are attached may form a heterocyclyl ring system as defined by HET-3;
HET-3 is an azetidine, pyrrolidine or piperidine ring, and is optionally substituted by methoxy, hydroxy or methyl;
R⁹ is halo, methyl or methoxy;
R¹⁰ is absent;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —S(O)ₚR⁴;

R³ is halo, methoxy or cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁹ is halo, methyl or methoxy;
R¹⁰ is absent;
p is independently at each occurrence 0, 1 or 2;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —S(O)ₚR⁴;

R³ is halo, methoxy or cyano;

### R⁴ is (1-4C)alkyl;

R⁹ is halo, methyl or methoxy;
R¹⁰ is absent;
p is independently at each occurrence 0, 1 or 2;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1;

### L is —O— or -(1-3C)alkylO-;

R¹ is (1-6C)alkyl, optionally substituted by a substituent selected from hydroxy and (1-4C)alkoxy;

### R² is HET-2;

R³ is halo, methoxy or cyano;
HET-2 is a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available nitrogen atom by a substituent selected from R⁶ and/or on an available carbon atom by 1 or 2 substituents independently selected from R⁷;
R⁶ is selected from (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁹ is halo, methyl or methoxy;
R¹⁰ is absent;
p is independently at each occurrence 0, 1 or 2;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is phenyl;

### L is —O— or -(1-3C)alkylO-;

R¹ is (1-6C)alkyl, optionally substituted by a substituent selected from hydroxy and (1-4C)alkoxy;
R² is selected from methylsulfonyl, ethylsulfonyl, methylsulfinyl, azetidinylcarbonyl, pyrrolidinylmethyl, dimethylaminocarbonyl, and oxadiazolyl;
R³ is selected from fluoro, chloro, cyano, methoxy and carboxy;
R⁹ is halo, methyl or methoxy;
R¹⁰ is absent;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1 wherein HET-1 is a fully unsaturated (aromatic) heterocyclic ring;

### L is —O— or -(1-3C)alkylO-;

R¹ is (1-6C)alkyl or HET-1 (wherein HET-1 is a saturated 5- or 6-membered heterocyclic ring, for example tetrahydrofuranyl or tetrahydropyranyl, particularly tetrahydrofuranyl), and R¹ is optionally substituted by a substituent selected from hydroxy and (1-4C)alkoxy;
R² is selected from —C(O)NR⁴R⁵, —S(O)ₚR⁴ and HET-2 (wherein HET-2 is selected from furyl, thienyl, thiazolyl, isothiazolyl, thiadiazolyl, pyrazolyl, imidazolyl, oxazolyl, isoxazolyl and oxadiazolyl; and HET-2 is optionally substituted on carbon by R⁷);
R³ is selected from fluoro, chloro, cyano, methoxy and carboxy;
R⁴ is selected from hydrogen, (1-4C)alkyl [optionally substituted by 1 or 2 substituents independently selected from HET-2 (wherein HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, 2,5-dioxopyrrolidinyl, tetrahydrothienyl, 1-oxotetrahydrothienyl, 1,1-dioxotetrahydrothienyl, 2-oxotetrahydrofuranyl, tetrahydrofuranyl, tetrahydropyranyl and 1,1-dioxothiomorpholino), OR⁵, cyano, —NR^{4′}R^{5′} and —C(O)NR⁵R⁵], (3-6C)cycloalkyl (optionally substituted with 1 group selected from R⁷), (2-4C)alkenyl, (2-4C)alkynyl and HET-2 (wherein HET-2 is selected from piperidinyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxotetrahydrofuranyl, tetrahydropyranyl, tetrahydropyranyl, 1,1-dioxothiomorpholino and 1,3-dioxolanyl);
R⁵ is (independently at each occurrence) selected from hydrogen, (1-4C)alkyl and (3-6C)cycloalkyl;
or R⁴ and R⁵ together with the nitrogen atom to which they are attached form a heterocyclyl ring system as defined by HET-3;
HET-3 is selected from morpholino, thiomorpholino (and versions thereof wherein the sulfur is oxidised to an SO or S(O)₂ group), piperidinyl, piperazinyl, pyrrolidinyl and azetidinyl; and HET-3 is optionally substituted by a substituent selected from hydroxy, methoxy, (1-4C)alkyl, allyl, trifluoromethyl, methylaminocarbonyl, dimethylaminocarbonyl and hydroxy(1-4C)alkyl (such as hydroxyethyl);
R⁴′ and R^{5′} are independently selected from hydrogen and (1-4C)alkyl;
R⁷ is selected from —OR⁵ (wherein R⁵ is hydrogen or (1-4C)alkyl) and hydroxy(1-4C)alkyl;
R⁹ is selected from halo, (1-4C)alkyl and cyano;
R¹⁰ is methoxy, methyl or halo;
p is (independently at each occurrence) 0, 1 or 2;
m is 0 or 1;
n is 0 or 1.

In a further aspect of the invention is provided a compound of the formula (IA) as hereinbefore defined or a pharmaceutically-acceptable salt or pro-drug thereof wherein:

Ring A is selected from phenyl and HET-1 wherein HET-1 is a fully unsaturated (aromatic) heterocyclic ring;

### L is —O— or -(1-3C)alkylO-;

R¹ is (1-6C)alkyl or HET-1 (wherein HET-1 is a saturated 5- or 6-membered heterocyclic ring, for example tetrahydrofuranyl or tetrahydropyranyl, particularly tetrahydrofuranyl), and R¹ is optionally substituted by a substituent selected from hydroxy and (1-4C)alkoxy;
R² is selected from —C(O)NR⁴R⁵, —S(O)ₚR⁴ and HET-2 (wherein HET-2 is selected from furyl, thienyl, thiazolyl, isothiazolyl, thiadiazolyl, pyrazolyl, imidazolyl, oxazolyl, isoxazolyl and oxadiazolyl; and HET-2 is optionally substituted on carbon by R⁷);
R³ is selected from fluoro, chloro, cyano, methoxy and carboxy;
R⁴ is selected from hydrogen, (1-4C)alkyl [optionally substituted by 1 or 2 substituents independently selected from HET-2 (wherein HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, 2,5-dioxopyrrolidinyl, tetrahydrothienyl, 1-oxotetrahydrothienyl, 1,1-dioxotetrahydrothienyl, 2-oxotetrahydrofuranyl, tetrahydrofuranyl, tetrahydropyranyl and 1,1-dioxothiomorpholino), —OR⁵, cyano, —NR^{4′}R^{5′} and —C(O)NR⁵R⁵], (3-6C)cycloalkyl (optionally substituted with 1 group selected from R⁷), (2-4C)alkenyl, (2-4C)alkynyl and HET-2 (wherein HET-2 is selected from piperidinyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxotetrahydrofuranyl, tetrahydrofuranyl, tetrahydropyranyl, 1,1-dioxothiomorpholino and 1,3-dioxolanyl);
R⁵ is (independently at each occurrence) selected from hydrogen, (1-4C)alkyl and (3-6C)cycloalkyl;
or R⁴ and R⁵ together with the nitrogen atom to which they are attached form a heterocyclyl ring system as defined by HET-3;
HET-3 is selected from morpholino, thiomorpholino (and versions thereof wherein the sulfur is oxidised to an SO or S(O)₂ group), piperidinyl, piperazinyl, pyrrolidinyl and azetidinyl; and HET-3 is optionally substituted by a substituent selected from hydroxy, methoxy, (1-4C)alkyl, allyl, trifluoromethyl, methylaminocarbonyl, dimethylaminocarbonyl and hydroxy(1-4C)alkyl (such as hydroxyethyl);
R⁴′ and R^{5′} are independently selected from hydrogen and (1-4C)alkyl;
R⁷ is selected from —OR⁵ (wherein R⁵ is hydrogen or (1-4C)alkyl) and hydroxy(1-4C)alkyl;
R⁹ is fluoro;
R¹⁰ is absent;
p is (independently at each occurrence) 0, 1 or 2;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1 wherein HET-1 is a fully unsaturated (aromatic) heterocyclic ring;

### L is —O— or —CH₂O—;

R¹ is (1-6C)alkyl or HET-1 wherein HET-1 is 5- or 6-membered fully saturated heterocyclic ring, for example tetrahydrofuranyl or tetrahydropyranyl, particularly tetrahydrofuranyl, and R¹ is optionally substituted by a substituent selected from hydroxy and (1-4C)alkoxy;
R² is selected from —C(O)NR⁴R⁵, —S(O)ₚR⁴ and HET-2 (wherein HET-2 is selected from furyl, thienyl, thiazolyl, isothiazolyl, thiadiazolyl, pyrazolyl, imidazolyl, oxazolyl, isoxazolyl and oxadiazolyl; and HET-2 is optionally substituted on carbon by R⁷);
R³ is selected from fluoro, chloro, cyano, methoxy and carboxy;
R⁴ is selected from hydrogen, (1-4C)alkyl [optionally substituted by 1 or 2 substituents independently selected from HET-2 (wherein HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, 2,5-dioxopyrrolidinyl, tetrahydrothienyl, 1-oxotetrahydrothienyl, 1,1-dioxotetrahydrothienyl, 2-oxotetrahydrofuranyl, tetrahydrofuranyl, tetrahydropyranyl and 1,1-dioxothiomorpholino), —OR⁵, cyano, —NR^{4′}R^{5′} and —C(O)NR⁵R⁵], (3-6C)cycloalkyl (optionally substituted with 1 group selected from R⁷), (2-4C)alkenyl, (2-4C)alkynyl and HET-2 (wherein HET-2 is selected from piperidinyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxotetrahydrofuranyl, tetrahydrofuranyl, tetrahydropyranyl, 1,1-dioxothiomorpholino and 1,3-dioxolanyl);
R⁵ is (independently at each occurrence) selected from hydrogen, (1-4C)alkyl and (3-6C)cycloalkyl;
or R⁴ and R⁵ together with the nitrogen atom to which they are attached form a heterocyclyl ring system as defined by HET-3;
HET-3 is selected from morpholino, thiomorpholino (and versions thereof wherein the sulfur is oxidised to an SO or S(O)₂ group), piperidinyl, piperazinyl, pyrrolidinyl and azetidinyl; and HET-3 is optionally substituted by a substituent selected from hydroxy, methoxy, (1-4C)alkyl, allyl, trifluoromethyl, methylaminocarbonyl, dimethylaminocarbonyl and hydroxy(1-4C)alkyl (such as hydroxyethyl);
R⁴′ and R^{5′} are independently selected from hydrogen and (1-4C)alkyl;
R⁷ is selected from —OR⁵ (wherein R⁵ is hydrogen or (1-4C)alkyl) and hydroxy(1-4C)alkyl;
R⁹ is selected from halo, (1-4C)alkyl and cyano;
R¹⁰ is methoxy, methyl or halo;
p is (independently at each occurrence) 0, 1 or 2;
m is 0 or 1, particularly 1;
n is 0 or 1.

In a further aspect of the invention is provided a compound of the formula (IA) as hereinbefore defined or a pharmaceutically-acceptable salt or pro-drug thereof wherein:

Ring A is selected from phenyl and HET-1 wherein HET-1 is a fully unsaturated (aromatic) heterocyclic ring;

### L is —O— or —CH₂O—;

R¹ is (1-6C)alkyl or HET-1 wherein HET-1 is 5- or 6-membered fully saturated heterocyclic ring, for example tetrahydrofuranyl or tetrahydropyranyl, particularly tetrahydrofuranyl, and R¹ is optionally substituted by a substituent selected from hydroxy and (1-4C)alkoxy;
R² is selected from —C(O)NR⁴R⁵, —S(O)ₚR⁴ and HET-2 (wherein HET-2 is selected from furyl, thienyl, thiazolyl, isothiazolyl, thiadiazolyl, pyrazolyl, imidazolyl, oxazolyl, isoxazolyl and oxadiazolyl; and HET-2 is optionally substituted on carbon by R⁷);
R³ is selected from fluoro, chloro, cyano, methoxy and carboxy;
R⁴ is selected from hydrogen, (1-4C)alkyl [optionally substituted by 1 or 2 substituents to independently selected from HET-2 (wherein HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, 2,5-dioxopyrrolidinyl, tetrahydrothienyl, 1-oxotetrahydrothienyl, 1,1-dioxotetrahydrothienyl, 2-oxotetrahydrofuranyl, tetrahydrofuranyl, tetrahydropyranyl and 1,1-dioxothiomorpholino), —OR⁵, cyano, —NR^{4′}R^{5′} and —C(O)NR⁵R⁵], (3-6C)cycloalkyl (optionally substituted with 1 group selected from R⁷), (2-4C)alkenyl, (2-4C)alkynyl and HET-2 (wherein HET-2 is selected from piperidinyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxotetrahydrofuranyl, tetrahydrofuranyl, tetrahydropyranyl, 1,1-dioxothiomorpholino and 1,3-dioxolanyl);
R⁵ is (independently at each occurrence) selected from hydrogen, (1-4C)alkyl and (3-6C)cycloalkyl;
or R⁴ and R⁵ together with the nitrogen atom to which they are attached form a heterocyclyl ring system as defined by HET-3;
HET-3 is selected from morpholino, thiomorpholino (and versions thereof wherein the sulfur is oxidised to an SO or S(O)₂ group), piperidinyl, piperazinyl, pyrrolidinyl and azetidinyl; and HET-3 is optionally substituted by a substituent selected from hydroxy, methoxy, (1-4C)alkyl, allyl, trifluoromethyl, methylaminocarbonyl, dimethylaminocarbonyl and hydroxy(1-4C)alkyl (such as hydroxyethyl);
R⁴′ and R^{5′} are independently selected from hydrogen and (1-4C)alkyl;
R⁷ is selected from —OR⁵ (wherein R⁵ is hydrogen or (1-4C)alkyl) and hydroxy(1-4C)alkyl;
R⁹ is selected from fluoro;
R¹⁰ is absent;
p is (independently at each occurrence) 0, 1 or 2;
m is 0 or 1, particularly 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1 wherein HET-1 is a fully unsaturated (aromatic) heterocyclic ring (suitably pyridyl or pyrazinyl);

### L is —O— or —CH₂O—;

R¹ is (1-6C)alkyl or HET-1 wherein HET-1 is 5- or 6-membered fully saturated heterocyclic ring, for example tetrahydrofuranyl or tetrahydropyranyl, particularly tetrahydrofuranyl, and R¹ is optionally substituted by a substituent selected from hydroxy and (1-4C)alkoxy;

R² is selected from —C(O)NR⁴R⁵, —S(O)ₚR⁴ and HET-2 (wherein HET-2 is selected from furyl, thienyl, thiazolyl, isothiazolyl, thiadiazolyl, pyrazolyl, imidazolyl, oxazolyl, isoxazolyl and oxadiazolyl; and HET-2 is optionally substituted on carbon by R⁷);

R³ is selected from fluoro, chloro, cyano, methoxy and carboxy;
R⁴ is selected from hydrogen, (1-4C)alkyl [optionally substituted by 1 or 2 substituents independently selected from HET-2 (wherein HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, 2,5-dioxopyrrolidinyl, tetrahydrothienyl, 1-oxotetrahydrothienyl, 1,1-dioxotetrahydrothienyl, 2-oxotetrahydrofuranyl, tetrahydrofuranyl, tetrahydropyranyl and 1,1-dioxothiomorpholino), —OR⁵, cyano, —NR^{4′}R^{5′} and —C(O)NR⁵R⁵], (3-6C)cycloalkyl (optionally substituted with 1 group selected from R⁷), (2-4C)alkenyl, (2-4C)alkynyl and HET-2 (wherein HET-2 is selected from piperidinyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxotetrahydrofuranyl, tetrahydrofuranyl, tetrahydropyranyl, 1,1-dioxothiomorpholino and 1,3-dioxolanyl);
R⁵ is (independently at each occurrence) selected from hydrogen, (1-4C)alkyl and (3-6C)cycloalkyl;
or R⁴ and R⁵ together with a nitrogen atom to which they are attached form a heterocyclyl ring system as defined by HET-3;
HET-3 is selected from morpholino, thiomorpholino (and versions thereof wherein the sulfur is oxidised to an SO or S(O)₂ group), piperidinyl, piperazinyl, pyrrolidinyl and azetidinyl; and HET-3 is optionally substituted by a substituent selected from hydroxy, methoxy, (1-4C)alkyl, allyl, trifluoromethyl, methylaminocarbonyl, dimethylaminocarbonyl and hydroxy(1-4C)alkyl (such as hydroxyethyl);
R⁴′ and R^{5′} are independently selected from hydrogen and (1-4C)alkyl;
R⁷ is selected from —OR⁵ (wherein R⁵ is hydrogen or (1-4C)alkyl) and hydroxy(1-4C)alkyl;
R⁹ and R¹⁰ are both absent, or one is absent and the other is halo, suitably fluoro;
p is (independently at each occurrence) 0, 1 or 2;
m is 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1 wherein HET-1 is a fully unsaturated (aromatic) heterocyclic ring (suitably pyridyl or pyrazinyl);

### L is —O— or —CH₂O—;

R¹ is (1-6C)alkyl or HET-1 wherein HET-1 is 5- or 6-membered fully saturated heterocyclic ring, for example tetrahydrofuranyl or tetrahydropyranyl, particularly tetrahydrofuranyl, and R¹ is optionally substituted by a substituent selected from hydroxy and (1-4C)alkoxy;

### R² is —C(O)NR⁴R⁵ or —S(O)ₚR⁴;

R³ is selected from fluoro, chloro, cyano, methoxy and carboxy;
R⁴ is selected from hydrogen, (1-4C)alkyl [optionally substituted by 1 or 2 substituents independently selected from HET-2 (wherein HET-2 is selected from furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, 2,5-dioxopyrrolidinyl, tetrahydrothienyl, 1-oxotetrahydrothienyl, 1,1-dioxotetrahydrothienyl, 2-oxotetrahydrofuranyl, tetrahydrofuranyl, tetrahydropyranyl and 1,1-dioxothiomorpholino), —OR⁵, cyano, —NR^{4′}R^{5′} and —C(O)NR⁵R⁵], (3-6C)cycloalkyl (optionally substituted with 1 group selected from R⁷), and HET-2 (wherein HET-2 is selected from piperidinyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxotetrahydrofuranyl, tetrahydrofuranyl, tetrahydropyranyl, 1,1-dioxothiomorpholino and 1,3-dioxolanyl);
R⁵ is (independently at each occurrence) selected from hydrogen, (1-4C)alkyl and (3-6C)cycloalkyl;
or R⁴ and R⁵ together with the nitrogen atom to which they are attached form a heterocyclyl ring system as defined by HET-3;
HET-3 is selected from morpholino, thiomorpholino (and versions thereof wherein the sulfur is oxidised to an SO or S(O)₂ group), piperidinyl, piperazinyl, pyrrolidinyl and azetidinyl; and HET-3 is optionally substituted by a substituent selected from hydroxy, methoxy, (1-4C)alkyl, allyl, trifluoromethyl, methylaminocarbonyl, dimethylaminocarbonyl and hydroxy(1-4C)alkyl (such as hydroxyethyl);
R⁴′ and R^{5′} are independently selected from hydrogen and (1-4C)alkyl;
R⁹ and R¹⁹ are both absent, or one is absent and the other is halo, suitably fluoro;
p is (independently at each occurrence) 0, 1 or 2;
m is 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl and HET-1 wherein HET-1 is a fully unsaturated (aromatic) heterocyclic ring;

### L is —O— or —CH₂O—;

R¹ is (1-6C)alkyl or HET-1 wherein HET-1 is 5- or 6-membered fully saturated heterocyclic ring, for example tetrahydrofuranyl or tetrahydropyranyl, particularly tetrahydrofuranyl, and R¹ is optionally substituted by a substituent selected from hydroxy and (1-4C)alkoxy;

### R² is —C(O)NR⁴R⁵ or —S(O)ₚ(1-4C)alkyl;

R³ is selected from fluoro, chloro, cyano, methoxy and carboxy;
R⁴ and R⁵ together with the nitrogen atom to which they are attached form a heterocyclyl ring system as defined by HET-3;
HET-3 is selected from morpholino, thiomorpholino (and versions thereof wherein the sulfur is oxidised to an SO or S(O)₂ group), piperidinyl, piperazinyl, pyrrolidinyl and azetidinyl; and HET-3 is optionally substituted by a substituent selected from hydroxy, methoxy, (1-4C)alkyl, allyl, trifluoromethyl, methylaminocarbonyl, dimethylaminocarbonyl and hydroxy(1-4C)alkyl (such as hydroxyethyl);
R⁹ and R¹⁰ are both absent, or one is absent and the other is halo, suitably fluoro;
p is (independently at each occurrence) 0, 1 or 2;
m is 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl, pyridyl and pyrazinyl;

### L is —O— or —CH₂O—;

R¹ is (1-6C)alkyl or HET-1 wherein HET-1 is 5- or 6-membered fully saturated heterocyclic ring, for example tetrahydrofuranyl or tetrahydropyranyl, particularly tetrahydrofuranyl, and R¹ is optionally substituted by a substituent selected from hydroxy and (1-4C)alkoxy;

### R² is —C(O)NR⁴R⁵ or —S(O)ₚ(1-4C)alkyl;

R³ is selected from fluoro, chloro, cyano, methoxy and carboxy;
R⁴ and R⁵ together with the nitrogen atom to which they are attached form a heterocyclyl ring system as defined by HET-3;
HET-3 is selected from morpholino, thiomorpholino (and versions thereof wherein the sulfur is oxidised to an SO or S(O)₂ group), piperidinyl, piperazinyl, pyrrolidinyl and azetidinyl; and HET-3 is optionally substituted by a substituent selected from hydroxy, methoxy, (1-4C)alkyl, allyl, trifluoromethyl, methylaminocarbonyl, dimethylaminocarbonyl and hydroxy(1-4C)alkyl (such as hydroxyethyl);
R⁹ and R¹⁹ are both absent, or one is absent and the other is halo, suitably fluoro;
p is (independently at each occurrence) 0, 1 or 2;
m is 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl, pyridyl and pyrazinyl;

### L is —O— or —CH₂O—;

R¹ is selected from hydroxyisopropyl, methoxyisopropyl, isopropyl, tert-butoxyisopropyl and tetrahydrofuran-2-yl;

### R² is —C(O)NR⁴R⁵ or —S(O)ₚ(1-4C)alkyl;

R³ is selected from fluoro, chloro, cyano, methoxy and carboxy;
R⁴ and R⁵ together with the nitrogen atom to which they are attached form a heterocyclyl ring system as defined by HET-3;
HET-3 is selected from morpholino, thiomorpholino (and versions thereof wherein the sulfur is oxidised to an SO or S(O)₂ group), piperidinyl, piperazinyl, pyrrolidinyl and azetidinyl; and HET-3 is optionally substituted by a substituent selected from hydroxy, methoxy, (1-4C)alkyl, allyl, trifluoromethyl, methylaminocarbonyl, dimethylaminocarbonyl and hydroxy(1-4C)alkyl (such as hydroxyethyl);
R⁹ and R¹⁰ are both absent, or one is absent and the other is halo, suitably fluoro;
p is (independently at each occurrence) 0, 1 or 2;
m is 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl, pyridyl, pyrimidinyl and pyrazinyl;

### L is —O— or -(1-3C)alkylO-;

R¹ is selected from hydroxyisopropyl, methoxyisopropyl, isopropyl, tert-butoxyisopropyl and tetrahydrofuran-2-yl;
R² is selected from methylsulfonyl, methylsulfonyl, ethylsulfonyl, prop-1-yn-3-ylaminocarbonyl, methylaminocarbonyl, dimethylaminocarbonyl, N-(cyanomethyl)-N-methylaminocarbonyl, cyclobutylaminocarbonyl, 1-(dimethylamino)ethylaminocarbonyl, methoxyethylaminocarbonyl, N-(hydroxyethyl)-N-methylaminocarbonyl, N-(N-methylpiperidin-4-yl)-N-(methyl)aminocarbonyl, aminocarbonylethylaminocarbonyl, 1-(dimethylamino)prop-2-ylaminocarbonyl, N-(hydroxyethyl)-N-(cyclobutyl)aminocarbonyl, pyrrolidinopropylamino, (2-hydroxytetrahydrothien-3-yl)aminocarbonyl, [4-(hydroxymethyptetrahydropyran-4-yl]aminocarbonyl, azetidinylcarbonyl, pyrrolidinylcarbonyl, (4-hydroxypiperidin-1-yl)carbonyl, (trifluoromethylpyrrolidinyl)carbonyl, N-methylpiperazinocarbonyl, 4-(methylaminocarbonyl)piperidin-1-ylcarbonyl, 4-(hydroxyethyl)piperazin-1-ylcarbonyl, 4-(isopropyl)piperazin-1-ylcarbonyl, (4-allyl)piperazin-1-ylcarbonyl, 1,1-dioxotetrahydrothien-3-ylaminocarbonyl, 1-(thien-2-yl)ethylaminocarbonyl, oxadiazolyl,
R³ is selected from fluoro, chloro, cyano, methoxy and carboxy;
R⁹ is selected from halo, (1-4C)alkyl and cyano;
R¹⁰ is methoxy, methyl or halo;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl, pyridyl and pyrazinyl;

### L is —O— or —CH₂O—;

R¹ is selected from hydroxyisopropyl, methoxyisopropyl, isopropyl, tert-butoxyisopropyl and tetrahydrofuran-2-yl;
R² is selected from methylsulfinyl, methylsulfonyl, ethylsulfonyl, prop-1-yn-3-ylaminocarbonyl, methylaminocarbonyl, dimethylaminocarbonyl, N-(cyanomethyl)-N-methylaminocarbonyl, cyclobutylaminocarbonyl, 1-(dimethylamino)ethylaminocarbonyl, methoxyethylaminocarbonyl, N-(hydroxyethyl)-N-methylaminocarbonyl, N-(N-methylpiperidin-4-yl)-N-(methyl)aminocarbonyl, aminocarbonylethylaminocarbonyl, 1-(dimethylamino)prop-2-ylaminocarbonyl, N-(hydroxyethyl)-N-(cyclobutyl)aminocarbonyl, pyrrolidinopropylamino, (2-hydroxytetrahydrothien-3-yl)aminocarbonyl, [4-(hydroxymethyptetrahydropyran-4-yl]aminocarbonyl, azetidinylcarbonyl, pyrrolidinylcarbonyl, (4-hydroxypiperidin-1-yl)carbonyl, (trifluoromethylpyrrolidinyl)carbonyl, N-methylpiperazinocarbonyl, 4-(methylaminocarbonyl)piperidin-1-ylcarbonyl, 4-(hydroxyethyl)piperazin-1-ylcarbonyl, 4-(isopropyl)piperazin-1-ylcarbonyl, (4-allyl)piperazin-1-ylcarbonyl, 1,1-dioxotetrahydrothien-3-ylaminocarbonyl, 1-(thien-2-yl)ethylaminocarbonyl, oxadiazolyl,
R³ is selected from fluoro, chloro, cyano, methoxy and carboxy;
R⁹ is selected from halo and cyano;
R¹⁰ is methoxy or halo;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl, pyridyl and pyrazinyl;

### L is —O— or —CH₂O—;

R¹ is selected from hydroxyisopropyl, methoxyisopropyl, isopropyl, tert-butoxyisopropyl and tetrahydrofuran-2-yl;
R² is selected from methylsulfinyl, methylsulfonyl, ethylsulfonyl, prop-1-yn-3-ylaminocarbonyl, methylaminocarbonyl, dimethylaminocarbonyl, N-(cyanomethyl)-N-methylaminocarbonyl, cyclobutylaminocarbonyl, 1-(dimethylamino)ethylaminocarbonyl, methoxyethylaminocarbonyl, N-(hydroxyethyl)-N-methylaminocarbonyl, N-(N-methylpiperidin-4-yl)-N-(methyl)aminocarbonyl, aminocarbonylethylaminocarbonyl, 1-(dimethylamino)prop-2-ylaminocarbonyl, N-(hydroxyethyl)-N-(cyclobutyl)aminocarbonyl, pyrrolidinopropylamino, (2-hydroxytetrahydrothien-3-yl)aminocarbonyl, [4-(hydroxymethyl)tetrahydropyran-4-yl]aminocarbonyl, azetidinylcarbonyl, pyrrolidinylcarbonyl, (4-hydroxypiperidin-1-yl)carbonyl, (trifluoromethylpyrrolidinyl)carbonyl, N-methylpiperazinocarbonyl, 4-(methylaminocarbonyl)piperidin-1-ylcarbonyl, 4-(hydroxyethyl)piperazin-1-ylcarbonyl, 4-(isopropyl)piperazin-1-ylcarbonyl, (4-allyl)piperazin-1-ylcarbonyl, 1,1-dioxotetrahydrothien-3-ylaminocarbonyl, 1-(thien-2-yl)ethylaminocarbonyl, oxadiazolyl,
R³ is selected from fluoro, chloro, cyano, methoxy and carboxy;
R⁹ is fluoro;
R¹⁰ is absent;
m is 0 or 1;
n is 0 or 1.

In a further aspect of the invention is provided a compound of the formula (I), (IA), (IB), and/or (IC) as hereinbefore defined or a pharmaceutically-acceptable salt or pro-drug thereof wherein:

Ring A is selected from phenyl, pyridyl and pyrazinyl;

### L is —O— or —CH₂O—;

R¹ is selected from hydroxyisopropyl, methoxyisopropyl, isopropyl, tert-butoxyisopropyl and tetrahydrofuran-2-yl;
R² is selected from methylsulfinyl, methylsulfonyl, ethylsulfonyl, prop-1-yn-3-ylaminocarbonyl, methylaminocarbonyl, dimethylaminocarbonyl, N-(cyanomethyl)-N-methylaminocarbonyl, cyclobutylaminocarbonyl, 1-(dimethylamino)ethylaminocarbonyl, methoxyethylaminocarbonyl, N-(hydroxyethyl)-N-methylaminocarbonyl, N-(N-methylpiperidin-4-yl)-N-(methyl)aminocarbonyl, aminocarbonylethylaminocarbonyl, 1-(dimethylamino)prop-2-ylaminocarbonyl, N-(hydroxyethyl)-N-(cyclobutyl)aminocarbonyl, pyrrolidinopropylamino, (2-hydroxytetrahydrothien-3-yl)aminocarbonyl, [4-(hydroxymethyptetrahydropyran-4-yl]aminocarbonyl, azetidinylcarbonyl, pyrrolidinylcarbonyl, (4-hydroxypiperidin-1-yl)carbonyl, (trifluoromethylpyrrolidinyl)carbonyl, N-methylpiperazinocarbonyl, 4-(methylaminocarbonyl)piperidin-1-ylcarbonyl, 4-(hydroxyethyl)piperazin-1-ylcarbonyl, 4-(isopropyl)piperazin-1-ylcarbonyl, (4-allyl)piperazin-1-ylcarbonyl, 1,1-dioxotetrahydrothien-3-ylaminocarbonyl, 1-(thien-2-yl)ethylaminocarbonyl, oxadiazolyl,
R³ is selected from fluoro, chloro, cyano, methoxy and carboxy;
R⁹ is selected from halo and cyano;
R¹⁰ is methoxy or halo;
m is 1;
n is 0 or 1.

In a further aspect of the invention is provided a compound of the formula (I), (IA), (IB), and/or (IC) (particularly (IA)) as hereinbefore defined or a pharmaceutically-acceptable salt or pro-drug thereof wherein:

Ring A is selected from phenyl, pyridyl and pyrazinyl;

### L is —O— or —CH₂O—;

R¹ is selected from hydroxyisopropyl, methoxyisopropyl, isopropyl, tert-butoxyisopropyl and tetrahydrofuran-2-yl;
R² is selected from methylsulfinyl, methylsulfonyl, ethylsulfonyl, prop-1-yn-3-ylaminocarbonyl, methylaminocarbonyl, dimethylaminocarbonyl, N-(cyanomethyl)-N-methylaminocarbonyl, cyclobutylaminocarbonyl, 1-(dimethylamino)ethylaminocarbonyl, methoxyethylaminocarbonyl, N-(hydroxyethyl)-N-methylaminocarbonyl, N-(N-methylpiperidin-4-yl)-N-(methyl)aminocarbonyl, aminocarbonylethylaminocarbonyl, 1-(dimethylamino)prop-2-ylaminocarbonyl, N-(hydroxyethyl)-N-(cyclobutyl)aminocarbonyl, pyrrolidinopropylamino, (2-hydroxytetrahydrothien-3-yl)aminocarbonyl, [4-(hydroxymethyl)tetrahydropyran-4-yl]aminocarbonyl, azetidinylcarbonyl, pyrrolidinylcarbonyl, (4-hydroxypiperidin-1-yl)carbonyl, (trifluoromethylpyrrolidinyl)carbonyl, N-methylpiperazinocarbonyl, 4-(methylaminocarbonyl)piperidin-1-ylcarbonyl, 4-(hydroxyethyl)piperazin-1-ylcarbonyl, 4-(isopropyl)piperazin-1-ylcarbonyl, (4-allyl)piperazin-1-ylcarbonyl, 1,1-dioxotetrahydrothien-3-ylaminocarbonyl, 1-(thien-2-yl)ethylaminocarbonyl, oxadiazolyl,
R³ is selected from fluoro, chloro, cyano, methoxy and carboxy;
R⁹ is fluoro;
R¹⁰ is absent;
m is 1;
n is 0 or 1.

Further preferred compounds of the invention are each of the Examples, each of which provides a further independent aspect of the invention. In further aspects, the present invention also comprises any two or more compounds of the Examples.

In one aspect, particular compounds of the invention comprise any one or more of:
2-{3-(benzyloxy)-5-[(1S)-2-methoxy-1-methylethoxy]phenyl}-3H-imidazo[4,5-b]pyridine;
8-{3-(benzyloxy)-5-[(1S)-2-methoxy-1-methylethoxy]phenyl}-9H-purine;
6-chloro-2-{3-[(1S)-2-methoxy-1-methylethoxy]-5-[4-(methylsulfonyl)phenoxy]phenyl}-3H-imidazo[4,5-b]pyridine;
8-{3-[(1S)-2-methoxy-1-methylethoxy]-5-[4-(methylsulfonyl)phenoxy]phenyl}-9H-purine;
2-{3-isopropoxy-5-[(1S)-1-methyl-2-phenylethoxy]phenyl}-3H-imidazo[4,5-b]pyridine;
6-bromo-2-{3-[(1S)-2-methoxy-1-methylethoxy]-5-[4-(methylsulfonyl)phenoxy]phenyl}-3H-imidazo[4,5-b]pyridine;
6-bromo-2-{3-[(1S)-2-methoxy-1-methylethoxy]-5-[4-(methylsulfonyl)phenoxy]phenyl}-3H-imidazo[4,5-b]pyridine;
2-{3-(benzyloxy)-5-[(1S)-2-methoxy-1-methylethoxy]phenyl}-5-bromo-1H-imidazo[4,5-b]pyrazine;
2-{3-[(1S)-2-methoxy-1-methylethoxy]-5-[4-(methylsulfonyl)phenoxy]phenyl}-3H-imidazo[4,5-b]pyridine;
2-{3-[4-(azetidin-1-ylcarbonyl)-2-fluorophenoxy]-5-[(1S)-2-methoxy-1-methylethoxy]phenyl}-3H-imidazo[4,5-b]pyridine;
3-chloro-4-{3-(3H-imidazo[4,5-b]pyridin-2-yl)-5-[(1S)-2-methoxy-1-methylethoxy]phenoxy}-N,N-dimethylbenzamide;
2-{3-[2-fluoro-4-(pyrrolidin-1-yl carbonyl)phenoxy]-5-[(1S)-2-methoxy-1-methylethoxy]phenyl}-3H-imidazo[4,5-b]pyridine;
2-{3-[2-chloro-4-(pyrrolidin-1-ylcarbonyl)phenoxy]-5-[(1S)-2-methoxy-1-methylethoxy]phenyl}-3H-imidazo[4,5-b]pyridine;
2-{3-[2-fluoro-4-(methylsulfonyl)phenoxy]-5-[(1S)-2-methoxy-1-methylethoxy]phenyl}-3H-imidazo[4,5-b]pyridine;
3-fluoro-4-{3-(3H-imidazo[4,5-b]pyridin-2-yl)-5-[(1S)-2-methoxy-1-methylethoxy]phenoxy}-N,N-dimethylbenzamide;
4-{3-(3H-imidazo[4,5-b]pyridin-2-yl)-5-[(1S)-2-methoxy-1-methylethoxy]phenoxy}benzonitrile;
2-{3-(3H-imidazo[4,5-b]pyridin-2-yl)-5-[(1S)-2-methoxy-1-methylethoxy]phenoxy}benzonitrile;
2-{3-isopropoxy-5-[4-(methylsulfonyl)phenoxy]phenyl}-3H-imidazo[4,5-b]pyridine;
3-{3-(3H-imidazo[4,5-b]pyridin-2-yl)-5-[(1S)-2-methoxy-1-methylethoxy]phenoxy}-N,N-dimethylbenzamide;
2-{3-[(1S)-2-methoxy-1-methylethoxy]-5-[3-(methylsulfinyl)phenoxy]phenyl}-3H-imidazo[4,5-b]pyridine;
4-({3-(3H-imidazo[4,5-b]pyridin-2-yl)-5-[(1S)-2-methoxy-1-methylethoxy]phenoxy}methyl)benzonitrile;
2-({3-(3H-imidazo[4,5-b]pyridin-2-yl)-5-[(1S)-2-methoxy-1-methylethoxy]phenoxy}methyl)benzonitrile;
2-{3-[(3-methoxybenzyl)oxy]-5-[(1S)-2-methoxy-1-methylethoxy]phenyl}-3H-imidazo[4,5-b]pyridine;
2-{3-[(2-fluorobenzyl)oxy]-5-[(1S)-2-methoxy-1-methylethoxy]phenyl}-3H-imidazo[4,5-b]pyridine;
2-(3-[(1S)-2-methoxy-1-methylethoxy]-5-{[4-(methylsulfonyl)benzyl]oxy}phenyl)-3H-imidazo[4,5-b]pyridine;
4-({3-(3H-imidazo[4,5-b]pyridin-2-yl)-5-[(1S)-2-methoxy-1-methylethoxy]phenoxy}methyl)-N,N-dimethylbenzamide;
(2S)-2-{3-(3H-imidazo[4,5-b]pyridin-2-yl)-5-[4-(methylsulfonyl)phenoxy]phenoxy}propan-1-ol; and
(2S)-2-{3-(6-bromo-3H-imidazo[4,5-b]pyridin-2-yl)-5-[4-(methylsulfonyl)phenoxy]phenoxy}propan-1-ol; and/or
2-{3-[(1S)-2-methoxy-1-methylethoxy]-5-[(1S)-1-methyl-2-phenylethoxy]phenyl}-3H-imidazo[4,5-b]pyridine;
2-{3-[(1S)-2-methoxy-1-methylethoxy]-5-[4-(methylsulfonyl)phenoxy]phenyl}-5-methyl-3H-imidazo[4,5-b]pyridine;
2-{3-[(1S)-2-methoxy-1-methylethoxy]-5-[4-(methylsulfonyl)phenoxy]phenyl}-6-methyl-3H-imidazo[4,5-b]pyridine;
2-{3-[(1S)-2-tert-butoxy-1-methylethoxy]-5-[4-(methylsulfonyl)phenoxy]phenyl}-5-methyl-3H-imidazo[4,5-b]pyridine;
2-{3-[(1S)-2-tert-butoxy-1-methylethoxy]-5-[4-(methylsulfonyl)phenoxy]phenyl}-6-methyl-3H-imidazo[4,5-b]pyridine;
2-{3-(benzyloxy)-5-[(1S)-2-methoxy-1-methylethoxy]phenyl}-5-methyl-3H-imidazo[4,5-b]pyridine;
(2S)-2-[3-[4-(azetidin-1-ylcarbonyl)-2-fluorophenoxy]-5-(3H-imidazo[4,5-b]pyridin-2-yl)phenoxy]propan-1-ol;
(2S)-2-[3-[4-(azetidin-1-ylcarbonyl)-2-fluorophenoxy]-5-(6-chloro-3H-imidazo[4,5-b]pyridin-2-yl)phenoxy]propan-1-ol;
8-{3-{[2-(azetidin-1-ylcarbonyl)pyrimidin-5-yl]oxy}-5-[(1S)-2-methoxy-1-methylethoxy]phenyl}-9H-purine;
5-{3-(3H-imidazo[4,5-b]pyridin-2-yl)-5-[(1S)-2-methoxy-1-methylethoxy]phenoxy}-N,N-dimethylpyrimidine-2-carboxamide;
5-[3-[(1S)-2-methoxy-1-methylethoxy]-5-(9H-purin-8-yl)phenoxy]-N,N-dimethylpyrimidine-2-carboxamide;
6-chloro-2-{3-[2-fluoro-4-(methylsulfonyl)phenoxy]-5-[(1S)-2-methoxy-1-methylethoxy]phenyl}-3H-imidazo[4,5-b]pyridine;
6-fluoro-2-{3-[(1S)-2-methoxy-1-methylethoxy]-5-[4-(methylsulfonyl)phenoxy]phenyl}-3H-imidazo[4,5-b]pyridine;
2-{3-{[5-(azetidin-1-ylcarbonyl)pyrazin-2-yl]oxy}-5-[(3S)-tetrahydrofuran-3-yloxy]phenyl}-1H-imidazo[4,5-b]pyridine;
8-{3-{[5-(azetidin-1-ylcarbonyl)pyrazin-2-yl]oxy}-5-[(3S)-tetrahydrofuran-3-yloxy]phenyl}-9H-purine;
8-{3-{[6-(azetidin-1-ylcarbonyl)pyridin-3-yl]oxy}-5-[(3S)-tetrahydrofuran-3-yloxy]phenyl}-9H-purine;
(2S)-2-{3-(6-chloro-3H-imidazo[4,5-b]pyridin-2-yl)-5-[4-(methylsulfonyl)phenoxy]phenoxy}propan-1-ol;
(2S)-2-{3-(5-methyl-3H-imidazo[4,5-b]pyridin-2-yl)-5-[4-(methylsulfonyl)phenoxy]phenoxy}propan-1-ol;
(2S)-2-(3-(3H-imidazo[4,5-b]pyridin-2-yl)-5-{4-[(4-methylpiperazin-1-yl)carbonyl]phenoxy}phenoxy)propan-1-ol;
(2S)-2-{3-(6-chloro-3H-imidazo[4,5-b]pyridin-2-yl)-5-[2-fluoro-4-(methylsulfonyl)phenoxy]phenoxy}propan-1-ol;
(2S)-2-{3-(6-fluoro-3H-imidazo[4,5-b]pyridin-2-yl)-5-[4-(methylsulfonyl)phenoxy]phenoxy}propan-1-ol;
5-[3-[(1S)-2-hydroxy-1-methylethoxy]-5-(9H-purin-8-yl)phenoxy]-N,N-dimethylpyrimidine-2-carboxamide;
5-[3-[(1S)-2-hydroxy-1-methylethoxy]-5-(9H-purin-8-yl)phenoxy]-N,N-dimethylpyrazine-2-carboxamide;
2-{3-[4-(ethylsulfonyl)-2-fluorophenoxy]-5-[(1S)-2-methoxy-1-methylethoxy]phenyl}-3H-imidazo[4,5-b]pyridine;
2-{3-[4-(azetidin-1-ylcarbonyl)-2-fluorophenoxy]-5-isopropoxyphenyl}-3H-imidazo[4,5-b]pyridine;
2-{3-[2-fluoro-4-(pyrrolidin-1-ylcarbonyl)phenoxy]-5-isopropoxyphenyl}-3H-imidazo[4,5-b]pyridine;
3-chloro-4-[3-(3H-imidazo[4,5-b]pyridin-2-yl)-5-isopropoxyphenoxy]-N,N-dimethylbenzamide;
5-chloro-6-{3-(3H-imidazo[4,5-b]pyridin-2-yl)-5-[(1S)-2-methoxy-1-methylethoxy]phenoxy}-N,N-dimethylnicotinamide;
5-methoxy-2-{3-[(1S)-2-methoxy-1-methylethoxy]-5-[4-(methylsulfonyl)phenoxy]phenyl}-1H-imidazo[4,5-b]pyrazine;
2-{3-[(1S)-2-methoxy-1-methylethoxy]-5-[4-(1,2,4-oxadiazol-3-yl)phenoxy]phenyl}-3H-imidazo[4,5-b]pyridine;
2-{3-[(1S)-2-methoxy-1-methylethoxy]-5-[4-(methylsulfonyl)phenoxy]phenyl}-3H-imidazo[4,5-b]pyridine-6-carbonitrile;
N-[2-(dimethylamino)ethyl]-6-{3-(3H-imidazo[4,5-b]pyridin-2-yl)-5-[(1S)-2-methoxy-1-methylethoxy]phenoxyl}nicotinamide;
4-{3-(3H-imidazo[4,5-b]pyridin-2-yl)-5-[(1S)-2-methoxy-1-methylethoxy]phenoxy}benzoic acid;
2-{3-[(1S)-2-methoxy-1-methylethoxy]-5-[4-(pyrrolidin-1-ylcarbonyl)phenoxy]phenyl}-3H-imidazo[4,5-b]pyridine;

2-[3-[(1S)-2-methoxy-1-methylethoxy]-5-(4-{[2-(trifluoromethyl)pyrrolidin-1-yl]carbonyl}phenoxy)phenyl]-3H-imidazo[4,5-b]pyridine;
4-{3-(3H-imidazo[4,5-b]pyridin-2-yl)-5-[(1S)-2-methoxy-1-methylethoxy]phenoxy}-N-[1-(2-thienyl)ethyl]benzamide;
N-(2-hydroxyethyl)-4-{3-(3H-imidazo[4,5-b]pyridin-2-yl)-5-[(1S)-2-methoxy-1-methylethoxy]phenoxy}-N-methylbenzamide;
4-{3-(3H-imidazo[4,5-b]pyridin-2-yl)-5-[(1S)-2-methoxy-1-methylethoxy]phenoxy}-N-methyl-N-(1-methylpiperidin-4-yl)benzamide;
N-(3-amino-3-oxopropyl)-4-{3-(3H-imidazo[4,5-b]pyridin-2-yl)-5-[(1S)-2-methoxy-1-methylethoxy]phenoxy}benzamide;
N-[4-(hydroxymethyl)tetrahydro-2H-pyran-4-yl]-4-{3-(3H-imidazo[4,5-b]pyridin-2-yl)-5-[(1S)-2-methoxy-1-methylethoxy]phenoxy}benzamide;
N-cyclobutyl-4-{3-(3H-imidazo[4,5-b]pyridin-2-yl)-5-[(1S)-2-methoxy-1-methylethoxy]phenoxy}benzamide;
4-{3-(3H-imidazo[4,5-b]pyridin-2-yl)-5-[(1S)-2-methoxy-1-methylethoxy]phenoxy}-N-prop-2-yn-1-ylbenzamide;
N-(1,1-dioxidotetrahydro-3-thienyl)-4-{3-(3H-imidazo[4,5-b]pyridin-2-yl)-5-[(1S)-2-methoxy-1-methylethoxy]phenoxy}benzamide;
1-(4-{3-(3H-imidazo[4,5-b]pyridin-2-yl)-5-[(1S)-2-methoxy-1-methylethoxy]phenoxy}benzoyl)-N-methylpiperidine-4-carboxamide;
2-[4-(4-{3-(3H-imidazo[4,5-b]pyridin-2-yl)-5-[(1S)-2-methoxy-1-methylethoxy]phenoxy}benzoyl)piperazin-1-yl]ethanol;
4-{3-(3H-imidazo[4,5-b]pyridin-2-yl)-5-[(1S)-2-methoxy-1-methylethoxy]phenoxy}-N-(2-methoxyethyl)benzamide;
1-(4-{3-(3H-imidazo[4,5-b]pyridin-2-yl)-5-[(1S)-2-methoxy-1-methylethoxy]phenoxy}benzoyl)piperidin-4-ol;
4-{3-(3H-imidazo[4,5-b]pyridin-2-yl)-5-[(1S)-2-methoxy-1-methylethoxy]phenoxy}-N-methylbenzamide;
2-{3-{4-[(4-isopropylpiperazin-1-yl)carbonyl]phenoxy}-5-[(1S)-2-methoxy-1-methylethoxy]phenyl}-3H-imidazo[4,5-b]pyridine;
N-(cyanomethyl)-4-{3-(3H-imidazo[4,5-b]pyridin-2-yl)-5-[(1S)-2-methoxy-1-methylethoxy]phenoxy}-N-methylbenzamide;
N-(4-hydroxytetrahydro-3-thienyl)-4-{3-(3H-imidazo[4,5-b]pyridin-2-yl)-5-[(1S)-2-methoxy-1-methylethoxy]phenoxy}benzamide;
N-cyclobutyl-N-(2-hydroxyethyl)-4-{3-(3H-imidazo[4,5-b]pyridin-2-yl)-5-[(1S)-2-methoxy-1-methylethoxy]phenoxy}benzamide;
2-{3-{4-[(4-allylpiperazin-1-yl)carbonyl]phenoxy}-5-[(1S)-2-methoxy-1-methylethoxy]phenyl}-3H-imidazo[4,5-b]pyridine;
4-{3-(3H-imidazo[4,5-b]pyridin-2-yl)-5-[(1S)-2-methoxy-1-methylethoxy]phenoxy}-N-(3-pyrrolidin-1-ylpropyl)benzamide;
N-[2-(dimethylamino)-1-methylethyl]-4-{3-(3H-imidazo[4,5-b]pyridin-2-yl)-5-[(1S)-2-methoxy-1-methylethoxy]phenoxy}benzamide;
4-{3-(3H-imidazo[4,5-b]pyridin-2-yl)-5-[(18)-2-methoxy-1-methylethoxy]phenoxy}-N,N-dimethylbenzamide;
2-(3-[(1S)-2-methoxy-1-methylethoxy]-5-{4-[(4-methylpiperazin-1-yl)carbonyl]phenoxy}phenyl)-3H-imidazo[4,5-b]pyridine; and
2-{3-[(1S)-2-methoxy-1-methylethoxy]-5-[4-(methylsulfonyl)phenoxy]phenyl}-1H-imidazo[4,5-b]pyrazine; and/or
2-{3-{[6-(azetidin-1-ylcarbonyl)pyridin-3-yl]oxy}-5-[(3S)-tetrahydrofuran-3-yloxy]phenyl}-6-fluoro-3H-imidazo[4,5-b]pyridine;
:N,N-dimethyl-5-({3-(9H-purin-8-yl)-5-[(3S)-tetrahydrofuran-3-yloxy]phenyl}oxy)pyrazine-2-carboxamide;
   or a pharmaceutically-acceptable salt or pro-drug thereof.

In another aspect, particular compounds of the invention comprise any one or more of:
2-{3-(benzyloxy)-5-[(1S)-2-methoxy-1-methylethoxy]phenyl}-3H-imidazo[4,5-b]pyridine;
8-{3-(benzyloxy)-5-[(1S)-2-methoxy-1-methylethoxy]phenyl}-9H-purine;
6-chloro-2-{3-[(1S)-2-methoxy-1-methylethoxy]-5-[4-(methylsulfonyl)phenoxy]phenyl}-3H-imidazo[4,5-b]pyridine;
8-{3-[(1S)-2-methoxy-1-methylethoxy]-5-[4-(methylsulfonyl)phenoxy]phenyl}-9H-purine;
6-bromo-2-{3-[(1S)-2-methoxy-1-methylethoxy]-5-[4-(methylsulfonyl)phenoxy]phenyl}-3H-imidazo[4,5-b]pyridine;
6-bromo-2-{3-[(1S)-2-methoxy-1-methylethoxy]-5-[4-(methylsulfonyl)phenoxy]phenyl}-3H-imidazo[4,5-b]pyridine;
2-{3-(benzyloxy)-5-[(1S)-2-methoxy-1-methylethoxy]phenyl}-5-bromo-1H-imidazo[4,5-b]pyrazine;
2-{3-[(1S)-2-methoxy-1-methylethoxy]-5-[4-(methylsulfonyl)phenoxy]phenyl}-3H-imidazo[4,5-b]pyridine;
2-{3-[4-(azetidin-1-ylcarbonyl)-2-fluorophenoxy]-5-[(1S)-2-methoxy-1-methylethoxy]phenyl}-3H-imidazo[4,5-b]pyridine;
3-chloro-4-{3-(3H-imidazo[4,5-b]pyridin-2-yl)-5-[(1S)-2-methoxy-1-methylethoxy]phenoxy}-N,N-dimethylbenzamide;
2-{3-[2-fluoro-4-(pyrrolidin-1-ylcarbonyl)phenoxy]-5-[(1S)-2-methoxy-1-methylethoxy]phenyl}-3H-imidazo[4,5-b]pyridine;
2-{3-[2-chloro-4-(pyrrolidin-1-ylcarbonyl)phenoxy]-5-[(1S)-2-methoxy-1-methylethoxy]phenyl}-3H-imidazo[4,5-b]pyridine;
2-{3-[2-fluoro-4-(methylsulfonyl)phenoxy]-5-[(1S)-2-methoxy-1-methylethoxy]phenyl}-3H-imidazo[4,5-b]pyridine;
3-fluoro-4-{3-(3H-imidazo[4,5-b]pyridin-2-yl)-5-[(1S)-2-methoxy-1-methylethoxy]phenoxy}-N,N-dimethylbenzamide;
4-{3-(3H-imidazo[4,5-b]pyridin-2-yl)-5-[(1S)-2-methoxy-1-methylethoxy]phenoxy}benzonitrile;
2-{3-(3H-imidazo[4,5-b]pyridin-2-yl)-5-[(1S)-2-methoxy-1-methylethoxy]phenoxy}benzonitrile;
2-{3-isopropoxy-5-[4-(methylsulfonyl)phenoxy]phenyl}-3H-imidazo[4,5-b]pyridine;
3-{3-(3H-imidazo[4,5-b]pyridin-2-yl)-5-[(1S)-2-methoxy-1-methylethoxy]phenoxy}-N,N-dimethylbenzamide;
2-{3-[(1S)-2-methoxy-1-methylethoxy]-5-[3-(methylsulfinyl)phenoxy]phenyl}-3H-imidazo[4,5-b]pyridine;
4-({3-(3H-imidazo[4,5-b]pyridin-2-yl)-5-[(1S)-2-methoxy-1-methylethoxy]phenoxy}methyl)benzonitrile;
2-({3-(3H-imidazo[4,5-b]pyridin-2-yl)-5-[(1S)-2-methoxy-1-methylethoxy]phenoxy}methyl)benzonitrile;
2-{3-[(3-methoxybenzyl)oxy]-5-[(1S)-2-methoxy-1-methylethoxy]phenyl}-3H-imidazo[4,5-b]pyridine;
2-{3-[(2-fluorobenzyl)oxy]-5-[(1S)-2-methoxy-1-methylethoxy]phenyl}-3H-imidazo[4,5-b]pyridine;
2-(3-[(1S)-2-methoxy-1-methylethoxy]-5-{[4-(methylsulfonyl)benzyl]oxy}phenyl)-3H-imidazo[4,5-b]pyridine;
4-({3-(3H-imidazo[4,5-b]pyridin-2-yl)-5-[(1S)-2-methoxy-1-methylethoxy]phenoxy}methyl)-N,N-dimethylbenzamide;
(2S)-2-{3-(3H-imidazo[4,5-b]pyridin-2-yl)-5-[4-(methylsulfonyl)phenoxy]phenoxy}propan-1-ol; and
(2S)-2-{3-(6-bromo-3H-imidazo[4,5-b]pyridin-2-yl)-5-[4-(methylsulfonyl)phenoxy]phenoxy}propan-1-ol; and/or
(2S)-2-[3-[4-(azetidin-1-ylcarbonyl)-2-fluorophenoxy]-5-(3H-imidazo[4,5-b]pyridin-2-yl)phenoxy]propan-1-ol;
(2S)-2-[3-[4-(azetidin-1-ylcarbonyl)-2-fluorophenoxy]-5-(6-chloro-3H-imidazo[4,5-b]pyridin-2-yl)phenoxy]propan-1-ol;
6-chloro-2-{3-[2-fluoro-4-(methylsulfonyl)phenoxy]-5-[(1S)-2-methoxy-1-methylethoxy]phenyl}-3H-imidazo[4,5-b]pyridine;
6-fluoro-2-{3-[(1S)-2-methoxy-1-methylethoxy]-5-[4-(methylsulfonyl)phenoxy]phenyl}-3H-imidazo[4,5-b]pyridine;
2-{3-{[5-(azetidin-1-ylcarbonyl)pyrazin-2-yl]oxy}-5-[(3S)-tetrahydrofuran-3-yloxy]phenyl}-1H-imidazo[4,5-b]pyridine;
8-{3-{[5-(azetidin-1-ylcarbonyl)pyrazin-2-yl]oxy}-5-[(3S)-tetrahydrofuran-3-yloxy]phenyl}-9H-purine;
8-{3-{[6-(azetidin-1-ylcarbonyl)pyridin-3-yl]oxy}-5-[(3S)-tetrahydrofuran-3-yloxy]phenyl}-9H-purine;
(2S)-2-{3-(6-chloro-3H-imidazo[4,5-b]pyridin-2-yl)-5-[4-(methylsulfonyl)phenoxy]phenoxy}propan-1-ol;
(2S)-2-{3-(5-methyl-3H-imidazo[4,5-b]pyridin-2-yl)-5-[4-(methylsulfonyl)phenoxy]phenoxy}propan-1-ol;
(2S)-2-(3-(3H-imidazo[4,5-b]pyridin-2-yl)-5-{4-[(4-methylpiperazin-1-yl)carbonyl]phenoxy}phenoxy)propan-1-ol;
(2S)-2-{3-(6-chloro-3H-imidazo[4,5-b]pyridin-2-yl)-5-[2-fluoro-4-(methylsulfonyl)phenoxy]phenoxy}propan-1-ol;
(2S)-2-{3-(6-fluoro-3H-imidazo[4,5-b]pyridin-2-yl)-5-[4-(methylsulfonyl)phenoxy]phenoxy}propan-1-ol;
5-[3-[(1S)-2-hydroxy-1-methylethoxy]-5-(9H-purin-8-yl)phenoxy]-N,N-dimethylpyrazine-2-carboxamide;
2-{3-[4-(ethylsulfonyl)-2-fluorophenoxy]-5-[(1S)-2-methoxy-1-methylethoxy]phenyl}-3H-imidazo[4,5-b]pyridine;
2-{3-[4-(azetidin-1-ylcarbonyl)-2-fluorophenoxy]-5-isopropoxyphenyl}-3H-imidazo[4,5-b]pyridine;
2-{3-[2-fluoro-4-(pyrrolidin-1-ylcarbonyl)phenoxy]-5-isopropoxyphenyl}-3H-imidazo[4,5-b]pyridine;
3-chloro-4-[3-(3H-imidazo[4,5-b]pyridin-2-yl)-5-isopropoxyphenoxy]-N,N-dimethylbenzamide;
5-chloro-6-{3-(3H-imidazo[4,5-b]pyridin-2-yl)-5-[(1S)-2-methoxy-1-methylethoxy]phenoxy}-N,N-dimethylnicotinamide;
5-methoxy-2-{3-[(1S)-2-methoxy-1-methylethoxy]-5-[4-(methylsulfonyl)phenoxy]phenyl}-1H-imidazo[4,5-b]pyrazine;
2-{3-[(1S)-2-methoxy-1-methylethoxy]-5-[4-(1,2,4-oxadiazol-3-yl)phenoxy]phenyl}-3H-imidazo[4,5-b]pyridine;
2-{3-[(1S)-2-methoxy-1-methylethoxy]-5-[4-(methylsulfonyl)phenoxy]phenyl}-3,1-imidazo[4,5-b]pyridine-6-carbonitrile;
N-[2-(dimethylamino)ethyl]-6-{3-(3H-imidazo[4,5-b]pyridin-2-yl)-5-[(1S)-2-methoxy-1-methylethoxy]phenoxy}nicotinamide;
4-{3-(3H-imidazo[4,5-b]pyridin-2-yl)-5-[(1S)-2-methoxy-1-methylethoxy]phenoxy}benzoic acid;
2-{3-[(1S)-2-methoxy-1-methylethoxy]-5-[4-(pyrrolidin-1-ylcarbonyl)phenoxy]phenyl}-3H-imidazo[4,5-b]pyridine;
2-[3-[(1S)-2-methoxy-1-methylethoxy]-5-(4-{[2-(trifluoromethyl)pyrrolidin-1-yl]carbonyl}phenoxy)phenyl]-3H-imidazo[4,5-b]pyridine;
4-{3-(3H-imidazo[4,5-b]pyridin-2-yl)-5-[(1S)-2-methoxy-1-methylethoxy]phenoxy}-N-[1-(2-thienyl)ethyl]benzamide;
N-(2-hydroxyethyl)-4-{3-(3H-imidazo[4,5-b]pyridin-2-yl)-5-[(1S)-2-methoxy-1-methylethoxy]phenoxy}-N-methylbenzamide;
4-{3-(3H-imidazo[4,5-b]pyridin-2-yl)-5-[(1S)-2-methoxy-1-methylethoxy]phenoxy}-N-methyl-N-(1-methylpiperidin-4-yl)benzamide;
N-(3-amino-3-oxopropyl)-4-{3-(3H-imidazo[4,5-b]pyridin-2-yl)-5-[(1S)-2-methoxy-1-methylethoxy]phenoxy}benzamide;
N-[4-(hydroxymethyl)tetrahydro-2H-pyran-4-yl]-4-{3-(3H-imidazo[4,5-b]pyridin-2-yl)-5-[(1S)-2-methoxy-1-methylethoxy]phenoxy}benzamide;
N-cyclobutyl-4-{3-(3H-imidazo[4,5-b]pyridin-2-yl)-5-[(1S)-2-methoxy-1-methylethoxy]phenoxy}benzamide;
4-{3-(3H-imidazo[4,5-b]pyridin-2-yl)-5-[(1S)-2-methoxy-1-methylethoxy]phenoxy}-N-prop-2-yn-1-ylbenzamide;
N-(1,1-dioxidotetrahydro-3-thienyl)-4-{3-(3H-imidazo[4,5-b]pyridin-2-yl)-5-[(1S)-2-methoxy-1-methylethoxy]phenoxy}benzamide;
1-(4-{3-(3H-imidazo[4,5-b]pyridin-2-yl)-5-[(1S)-2-methoxy-1-methylethoxy]phenoxy}benzoyl)-N-methylpiperidine-4-carboxamide;
2-[4-(4-{3-(3H-imidazo[4,5-b]pyridin-2-yl)-5-[(1S)-2-methoxy-1-methylethoxy]phenoxy}benzoyl)piperazin-1-yl]ethanol;
4-{3-(3H-imidazo[4,5-b]pyridin-2-yl)-5-[(1S)-2-methoxy-1-methylethoxy]phenoxy}-N-(2-methoxyethyl)benzamide;
1-(4-{3-(3H-imidazo[4,5-b]pyridin-2-yl)-5-[(1S)-2-methoxy-1-methylethoxy]phenoxy}benzoyl)piperidin-4-ol;
4-{3-(3H-imidazo[4,5-b]pyridin-2-yl)-5-[(1S)-2-methoxy-1-methylethoxy]phenoxy}-N-methylbenzamide;
2-{3-{4-[(4-isopropylpiperazin-1-yl)carbonyl]phenoxy}-5-[(1S)-2-methoxy-1-methylethoxy]phenyl}-3H-imidazo[4,5-b]pyridine;
N-(cyanomethyl)-4-{3-(3H-imidazo[4,5-b]pyridin-2-yl)-5-[(1S)-2-methoxy-1-methylethoxy]phenoxy}-N-methylbenzamide;
N-(4-hydroxytetrahydro-3-thienyl)-4-{3-(3H-imidazo[4,5-b]pyridin-2-yl)-5-[(1S)-2-methoxy-1-methylethoxy]phenoxy}benzamide;
N-cyclobutyl-N-(2-hydroxyethyl)-4-{3-(3H-imidazo[4,5-b]pyridin-2-yl)-5-[(1S)-2-methoxy-1-methylethoxy]phenoxy}benzamide;
2-{3-{4-[(4-allylpiperazin-1-yl)carbonyl]phenoxy}-5-[(1S)-2-methoxy-1-methylethoxy]phenyl}-3H-imidazo[4,5-b]pyridine;
4-{3-(3H-imidazo[4,5-b]pyridin-2-yl)-5-[(1S)-2-methoxy-1-methylethoxy]phenoxy}-N-(3-pyrrolidin-1-ylpropyl)benzamide;
N-[2-(dimethylamino)-1-methylethyl]-4-{3-(3H-imidazo[4,5-b]pyridin-2-yl)-5-[(1S)-2-methoxy-1-methylethoxy]phenoxy}benzamide;
4-{3-(3H-imidazo[4,5-b]pyridin-2-yl)-5-[(1S)-2-methoxy-1-methylethoxy]phenoxy}-N,N-dimethylbenzamide;
2-(3-[(1S)-2-methoxy-1-methylethoxy]-5-{4-[(4-methylpiperazin-1-yl)carbonyl]phenoxy}phenyl)-3H-imidazo[4,5-b]pyridine; and
2-{3-[(1S)-2-methoxy-1-methylethoxy]-5-[4-(methylsulfonyl)phenoxy]phenyl}-1H-imidazo[4,5-b]pyrazine;
   or a pharmaceutically-acceptable salt or pro-drug thereof.

In another aspect, particular compounds of the invention comprise any one or more of:
2-{3-[4-(ethylsulfonyl)-2-fluorophenoxy]-5-[(1S)-2-methoxy-1-methylethoxy]phenyl}-3H-imidazo[4,5-b]pyridine;
2-{3-[(1S)-2-methoxy-1-methylethoxy]-5-[(1S)-1-methyl-2-phenylethoxy]phenyl}-3H-imidazo[4,5-b]pyridine;
4-{3-(3H-imidazo[4,5-b]pyridin-2-yl)-5-[(1S)-2-methoxy-1-methylethoxy]phenoxy}benzoic acid;
(2S)-2-{3-(6-chloro-3H-imidazo[4,5-b]pyridin-2-yl)-5-[4-(methylsulfonyl)phenoxy]phenoxy}propan-1-ol;
2-{3-[(1S)-2-methoxy-1-methylethoxy]-5-[4-(methylsulfonyl)phenoxy]phenyl}-1H-imidazo[4,5-b]pyrazine;
2-{3-[4-(azetidin-1-ylcarbonyl)-2-fluorophenoxy]-5-isopropoxyphenyl}-3H-imidazo[4,5-b]pyridine;
5-methoxy-2-{3-[(1S)-2-methoxy-1-methylethoxy]-5-[4-(methylsulfonyl)phenoxy]phenyl}-1H-imidazo[4,5-b]pyrazine;
2-{3-[2-fluoro-4-(pyrrolidin-1-ylcarbonyl)phenoxy]-5-isopropoxyphenyl}-3H-imidazo[4,5-b]pyridine;
3-chloro-4-[3-(3H-imidazo[4,5-b]pyridin-2-yl)-5-isopropoxyphenoxy]-N,N-dimethylbenzamide;
2-{3-[(1S)-2-methoxy-1-methylethoxy]-5-[4-(methylsulfonyl)phenoxy]phenyl}-5-methyl-3H-imidazo[4,5-b]pyridine;
2-{3-[(1S)-2-methoxy-1-methylethoxy]-5-[4-(methylsulfonyl)phenoxy]phenyl}-6-methyl-3H-imidazo[4,5-b]pyridine;
2-{3-[(1S)-2-tert-butoxy-1-methylethoxy]-5-[4-(methylsulfonyl)phenoxy]phenyl}-5-methyl-3H-imidazo[4,5-b]pyridine;
2-{3-[(1S)-2-tert-butoxy-1-methylethoxy]-5-[4-(methylsulfonyl)phenoxy]phenyl}-6-methyl-3H-imidazo[4,5-b]pyridine;
2-{3-[(1S)-2-methoxy-1-methylethoxy]-5-[4-(pyrrolidin-1-ylcarbonyl)phenoxy]phenyl}-3H-imidazo[4,5-b]pyridine; and
2-{3-[(1S)-2-methoxy-1-methylethoxy]-5-[4-(1,2,4-oxadiazol-3-yl)phenoxy]phenyl}-3H-imidazo[4,5-b]pyridine;
   or a pharmaceutically-acceptable salt or pro-drug thereof.

In a further aspect, particular compounds of the invention comprise any one or is more of:
6-fluoro-2-{3-[(1S)-2-methoxy-1-methylethoxy]-5-[4-(methylsulfonyl)phenoxy]phenyl}-3H-imidazo[4,5-b]pyridine;
(2S)-2-{3-(6-fluoro-3H-imidazo[4,5-b]pyridin-2-yl)-5-[4-(methylsulfonyl)phenoxy]phenoxy}propan-1-ol; and
2-{3-{[6-(azetidin-1-ylcarbonyl)pyridin-3-yl]oxy}-5-[(3S)-tetrahydrofuran-3-yloxy]phenyl}-6-fluoro-3H-imidazo[4,5-b]pyridine;
   or a pharmaceutically-acceptable salt or pro-drug thereof.

The compounds of the invention may be administered in the form of a pro-drug. A pro-drug is a bioprecursor or pharmaceutically acceptable compound being degradable in the body to produce a compound of the invention (such as an ester or amide of a compound of the invention, particularly an in-vivo hydrolysable ester). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen;

### c) H. Bundgaard, Chapter 5 “Design and Application of Prodrugs”, by H. Bundgaard p. 113-191 (1991);

### d) H. Bundgaard, Advanced Drug Delivery Reviews, 8, 1-38 (1992);

### e) H. Bundgaard, et al., Journal of Pharmaceutical Sciences, 77, 285 (1988); and

### f) N. Kakeya, et al., Chem Pharm Bull, 32, 692 (1984).

The contents of the above cited documents are incorporated herein by reference.

Examples of pro-drugs are as follows. An in-vivo hydrolysable ester of a compound of the invention containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁ to C₆alkoxymethyl esters for example methoxymethyl, C₁ to C₆alkanoyloxymethyl esters for example pivaloyloxymethyl, phthalidyl esters, C₃ to C₈cycloalkoxycarbonyloxyC₁ to C₆alkyl esters for example 1-cyclohexylcarbonyloxyethyl; 1,3-dioxolen-2-onylmethyl esters, for example 5-methyl-1,3-dioxolen-2-onylmethyl; and (1-6C)alkoxycarbonyloxyethyl esters.

An in-vivo hydrolysable ester of a compound of the invention containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a benzoxazinone derivative of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

A further feature of the invention is a pharmaceutical composition comprising a compound of Formula (I), (IA), (IB) or (IC) as defined above, or a salt or prodrug thereof, together with a pharmaceutically-acceptable diluent or carrier.

According to another aspect of the invention there is provided a compound of Formula (I), (IA), (IB) or (IC) as defined above or a salt or prodrug thereof for use as a medicament.

According to another aspect of the invention there is provided a compound of Formula (I), (IA), (IB) or (IC) as defined above or a salt or prodrug thereof for use as a medicament for treatment of a disease mediated through GLK, in particular type 2 diabetes.

Further according to the invention there is provided the use of a compound of Formula (I), (IA), (IB) or (IC) or a salt or prodrug thereof in the preparation of a medicament for treatment of a disease mediated through GLK, in particular type 2 diabetes.

The compound is suitably formulated as a pharmaceutical composition for use in this way.

According to another aspect of the present invention there is provided a method of treating GLK mediated diseases, especially diabetes, by administering an effective amount of a compound of Formula (I), (IA), (IB) or (IC) or salt, or pro-drug thereof, to a mammal in need of such treatment.

Specific diseases which may be treated by a compound or composition of the invention include: blood glucose lowering in Type 2 Diabetes Mellitus without a serious risk of hypoglycaemia (and potential to treat type 1), dyslipidemia, obesity, insulin resistance, metabolic syndrome X, impaired glucose tolerance.

As discussed above, thus the GLK/GLKRP system can be described as a potential “Diabesity” target (of benefit in both Diabetes and Obesity). Thus, according to another aspect of the invention there is provided the use of a compound of Formula (I), (IA), (IB) or (IC) or salt or pro-drug thereof, in the preparation of a medicament for use in the combined treatment or prevention of diabetes and obesity.

According to another aspect of the invention there is provided the use of a compound of Formula (I), (IA), (IB) or (IC) or salt or pro-drug thereof, in the preparation of a medicament for use in the treatment of diabetes and obesity.

According to another aspect of the invention there is provided the use of a compound of Formula (I), (IA), (IB) or (IC) or salt or pro-drug thereof, in the preparation of a medicament for use in the treatment or prevention of obesity.

According to another aspect of the invention there is provided a compound of Formula (I), (IA), (IB) or (IC) as defined above or a salt or prodrug thereof for use as a medicament for treatment or prevention, particularly treatment of diabetes and obesity, in particular type 2 diabetes.

According to a further aspect of the invention there is provided a method for the combined treatment of obesity and diabetes by administering an effective amount of a compound of Formula (I), (IA), (IB) or (IC) or salt or pro-drug thereof, to a mammal in need of such treatment.

According to a further aspect of the invention there is provided a method for the treatment of obesity by administering an effective amount of a compound of Formula (I), (IA), (IB) or (IC) or salt or pro-drug thereof, to a mammal in need of such treatment.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). Dosage forms suitable for oral use are preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, to calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the Formula (I), (IA), (IB) or (IC) will naturally vary according to the nature and severity of the conditions, the age and sex of the animal or patient and the route of administration, according to well known principles of medicine.

In using a compound of the Formula (I), (IA), (IB) or (IC) for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

The elevation of GLK activity described herein may be applied as a sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets. For example in the treatment of diabetes mellitus, chemotherapy may include the following main categories of treatment:

1) Insulin and insulin analogues;
2) Insulin secretagogues including sulphonylureas (for example glibenclamide, glipizide), prandial glucose regulators (for example repaglinide, nateglinide);
3) Agents that improve incretin action (for example dipeptidyl peptidase IV inhibitors, and GLP-1 agonists);
4) Insulin sensitising agents including PPARgamma agonists (for example pioglitazone and rosiglitazone), and agents with combined PPARalpha and gamma activity;
5) Agents that modulate hepatic glucose balance (for example metformin, fructose 1, 6 bisphosphatase inhibitors, glycogen phopsphorylase inhibitors, glycogen synthase kinase inhibitors);
6) Agents designed to reduce the absorption of glucose from the intestine (for example acarbose);
7) Agents that prevent the reabsorption of glucose by the kidney (SGLT inhibitors);
8) Agents designed to treat the complications of prolonged hyperglycaemia (for example aldose reductase inhibitors);
9) Anti-obesity agents (for example sibutramine and orlistat);
10) Anti-dyslipidaemia agents such as, HMG-CoA reductase inhibitors (eg statins); PPARα agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
11) Antihypertensive agents such as, β blockers (eg atenolol, inderal); ACE inhibitors (eg lisinopril); Calcium antagonists (eg. nifedipine); Angiotensin receptor antagonists (eg candesartan), α antagonists and diuretic agents (eg. furosemide, benzthiazide);
12) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin;
13) Agents which antagonise the actions of glucagon; and
14) Anti-inflammatory agents, such as non-steroidal anti-inflammatory drugs (eg. aspirin) and steroidal anti-inflammatory agents (eg. cortisone).

According to another aspect of the present invention there is provided individual compounds produced as end products in the Examples set out below and salts and pro-drugs thereof.

In the above other pharmaceutical composition, process, method, use and medicament manufacture features, the alternative and preferred embodiments of the compounds of the invention described herein also apply.

A compound of the invention, or a salt thereof, may be prepared by any process known to be applicable to the preparation of such compounds or structurally related compounds. Functional groups may be protected and deprotected using conventional methods. For examples of protecting groups such as amino and carboxylic acid protecting groups (as well as means of formation and eventual deprotection), see T. W. Greene and P. G. M. Wuts, “Protective Groups in Organic Synthesis”, Second Edition, John Wiley & Sons, New York, 1991.

Processes for the synthesis of compounds of Formula (I), (IA), (IB) or (IC) are provided as a further feature of the invention. Thus, according to a further aspect of the invention there is provided a process for the preparation of a compound of Formula (I), which comprises a process a) to f) (wherein the variables are as defined hereinbefore for compounds of Formula (I) unless otherwise defined):

(a) reaction of a compound of Formula (III) with a compound of Formula (IV),

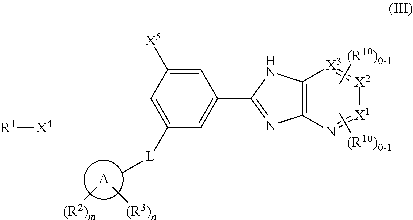

wherein X⁴ is a leaving group or an organometallic reagent and X⁵ is a hydroxyl group, or X⁴ is a hydroxyl group and X⁵ is a leaving group or an organometallic reagent, and wherein R¹ is as defined for a compound of formula (I), or is a protected version thereof; (b) reaction of a compound of Formula (V) with a compound of Formula (VI)

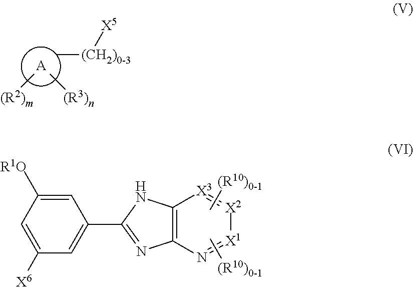

wherein X⁶ is a leaving group or an organometallic reagent and X⁷ is a hydroxyl group, or X⁶ is a hydroxyl group and X⁷ is a leaving group or an organometallic reagent, and wherein R¹ is as defined for a compound of formula (I), or a protected version thereof;

or

(c) reaction of a compound of Formula (VII) with a compound of Formula (VIII),

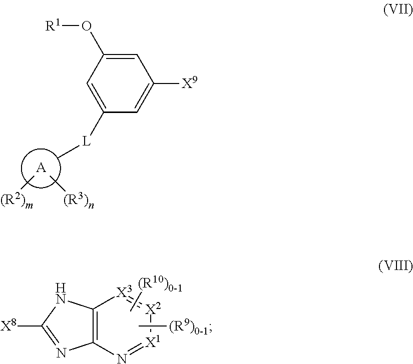

wherein X⁸ is a leaving group and X⁹ is an organometallic agent, or X⁸ is a leaving group and X⁹ is an organometallic agent; and wherein R¹ is as defined for a compound of formula (I) or a protected version thereof;
or
(d) reaction of a compound of formula (IX) with a compound of formula (X) and cyclisation in a one or two step reaction;

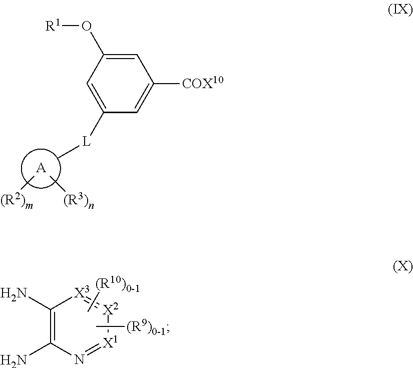

wherein X¹⁰ is hydrogen, a hydroxyl group, a halogen, or other leaving group, eg. —OR (wherein —OR represents an ester or activated ester), and wherein R¹ is as defined for a compound of formula (I) or a protected version thereof;
or
e) reaction of a compound of formula (XI) with a compound of formula (XII) and cyclisation in a one step reaction,

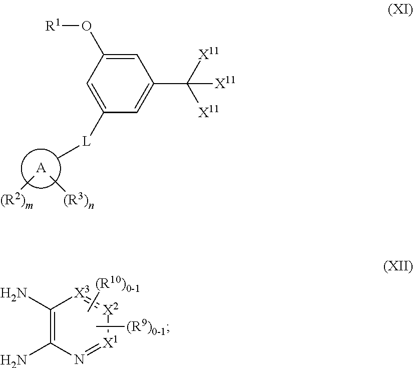

wherein each X¹¹ is a leaving group, preferably of the type O-methyl or O-ethyl, and wherein R¹ is as defined for a compound of formula (I) or a protected version thereof;
or
f) reaction of a compound of formula (XIII) with a compound of formula (XIV) and cyclisation in a one or two step reaction,

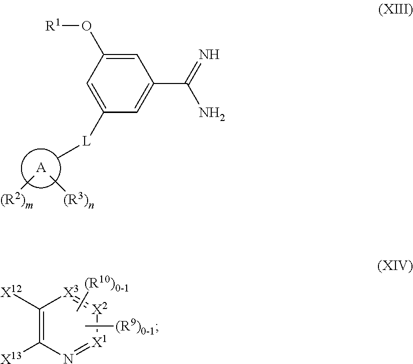

wherein X¹² and X¹³ are independently halogen or other leaving group, and wherein R¹ is as defined for a compound of formula (I) or a protected version thereof;
and thereafter, if necessary:
i) converting a compound of Formula (I) into another compound of Formula (I);
ii) removing any protecting groups; and/or
iii) forming a salt or pro-drug thereof.

Suitable leaving groups X¹ to X¹⁰ for processes a) to f) are any leaving groups known in the art for these types of reactions, for example halo, alkoxy, trifluoromethanesulfonyloxy, methanesulfonyloxy, p-toluenesulfonyloxy, or an organometallic moiety; or a group (such as a hydroxy group) that may be converted into a leaving group (such as an oxytriphenylphosphonium group) in situ.

Compounds of Formulae (III) to (XIV) are commercially available, or are known in the art, or may be made by processes known in the art, for example as shown in the accompanying Examples. For further information on processes for making such compounds, we refer to our PCT publications WO 03/000267, WO 03/015774 and WO 03/000262 and references therein.

Examples of conversions of a compound of Formula (I) into another compound of Formula (I), well known to those skilled in the art, include functional group interconversions such as hydrolysis, hydrogenation, hydrogenolysis, oxidation or reduction, and/or further functionalisation by standard reactions such as amide or metal-catalysed coupling, or nucleophilic displacement reactions;

Specific reaction conditions for the above reactions are as follows:

Processes a and b)—nucleophilic substitution reactions of alcohols or phenols (or, preferably, their anionic forms) with a suitable electrophile are well known in the art. For example,
(i) using an appropriate substitution reaction, such as an alkoxide with an aryl halide or triflate in a suitable solvent such as dimethylformamide (DMF), dimethylacetamide (DMA), N-methylpyrrolidone (NMP), or dimethyl sulfoxide (DMSO), at a temperature in the range 0 to 200° C., optionally using microwave heating, and optionally using metal catalysis such as palladium (II), palladium (0), copper (II) or copper (I); or (ii) using an appropriate substitution reaction, such as a phenoxide with an alkyl halide or triflate in a suitable solvent such as dimethylformamide (DMF), dimethylacetamide (DMA), N-methylpyrrolidone (NMP), or dimethyl sulfoxide (DMSO), at a temperature in the range 0 to 200° C., optionally using microwave heating, and optionally using metal catalysis such as palladium (II), palladium (0), copper (II) or copper (I); or
Process c)—compounds of Formula (VII) and (VIII) can be reacted together in a suitable solvent, such as DMF, THF or toluene, with a base such as sodium carbonate, potassium carbonate, or potassium tert-butoxide, at a temperature in the range 0 to 200° C., optionally using microwave heating or metal catalysis such as palladium(II), palladium(0), copper(II) or copper(I);
Process d)—reaction of a compound of Formula (IX) with a compound of Formula (X) can be performed in a one or two step reaction, exemplified but not limited by the following procedures:
i) in a polar solvent, such as DMF or a non-polar solvent such as THF with a peptide coupling agent such as EDAC, optionally with a base or bases such as triethylamine, DIPEA, or DMAP, followed by cyclisation at a temperature between 100° C. and 200° C., optionally using microwave heating and acid catalysis;
ii) from an acid chloride, followed by cyclisation at a temperature between 100° C. and 200° C., optionally using microwave heating and acid catalysis;
iii) in a one step procedure from a carboxylic acid and a coupling reagent such as carbonyl di-imidazole (CDI), followed by cyclisation at a temperature between 100° C. and 200° C., optionally using microwave heating and acid catalysis;
iv) in a one step procedure from a carboxylic acid and an acidic reagent such as polyphosphoric acid (PPA) at a temperature between 100° C. and 200° C.;
v) reaction between an appropriate aldehyde and diaminoheterocycle, in a polar or non-polar solvent such as DMF or acetonitrile, followed by an oxidation procedure using an oxidant such as atmospheric oxygen or metallic reagent such as an Fe(III) salt, at a temperature between 0° C. and 200° C., optionally using microwave heating;
Process e)—reaction of a compound of Formula (XI) with a compound of Formula (XII) can be performed in a one step reaction in a polar or non-polar solvent, preferably in the presence of an acid catalyst, at a temperature between 100° C. and 200° C., optionally using microwave heating;
Process f)—reaction of a compound of Formula (XIII) with a compound of Formula (XIV) can be performed in a one step reaction in a polar or non-polar solvent, at a temperature between 0° C. and 200° C., optionally using microwave heating;

Certain intermediates of formula (III), (IV), (V), (VI), (VII), (VIII), (IX), (X), (XI), (XII), (XIII) and/or (XIV) are believed to be novel and comprise an independent aspect of the invention.

During the preparation process, it may be advantageous to use a protecting group for a functional group within the molecule. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower” signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned is of course within the scope of the invention.

A carboxy protecting group may be the residue of an ester-forming aliphatic or araliphatic alcohol or of an ester-forming silanol (the said alcohol or silanol preferably containing 1-20 carbon atoms). Examples of carboxy protecting groups include straight or branched chain (1-12C)alkyl groups (e.g. isopropyl, t-butyl); lower alkoxy lower alkyl groups (e.g. methoxymethyl, ethoxymethyl, isobutoxymethyl; lower aliphatic acyloxy lower alkyl groups, (e.g. acetoxymethyl, propionyloxymethyl, butyryloxymethyl, pivaloyloxymethyl); lower alkoxycarbonyloxy lower alkyl groups (e.g. 1-methoxycarbonyloxyethyl, 1-ethoxycarbonyloxyethyl); aryl lower alkyl groups (e.g. p-methoxybenzyl, o-nitrobenzyl, p-nitrobenzyl, benzhydryl and phthalidyl); tri(lower alkyl)silyl groups (e.g. trimethylsilyl and t-butyldimethylsilyl); tri(lower alkyl)silyl lower alkyl groups (e.g. trimethylsilylethyl); and (2-6C)alkenyl groups (e.g. allyl and vinylethyl).

Methods particularly appropriate for the removal of carboxyl protecting groups include for example acid-, metal- or enzymically-catalysed hydrolysis.

Examples of hydroxy protecting groups include methyl, t-butyl, lower alkenyl groups (e.g. allyl); lower alkanoyl groups (e.g. acetyl); lower alkoxycarbonyl groups (e.g. t-butoxycarbonyl); lower alkenyloxycarbonyl groups (e.g. allyloxycarbonyl); aryl lower alkoxycarbonyl groups (e.g. benzoyloxycarbonyl, p-methoxybenzyloxycarbonyl, o-nitrobenzyloxycarbonyl, p-nitrobenzyloxycarbonyl); tri lower alkyl/arylsilyl groups (e.g. trimethylsilyl, t-butyldimethylsilyl, t-butyldiphenylsilyl); tetrahydropyran-2-yl; aryl lower alkyl groups (e.g. benzyl) groups; and triaryl lower alkyl groups (e.g. triphenylmethyl).

Examples of amino protecting groups include formyl, aralkyl groups (e.g. benzyl and substituted benzyl, e.g. p-methoxybenzyl, nitrobenzyl and 2,4-dimethoxybenzyl, and triphenylmethyl); di-p-anisylmethyl and furylmethyl groups; lower alkoxycarbonyl (e.g. t-butoxycarbonyl); lower alkenyloxycarbonyl (e.g. allyloxycarbonyl); aryl lower alkoxycarbonyl groups (e.g. benzyloxycarbonyl, p-methoxybenzyloxycarbonyl, o-nitrobenzyloxycarbonyl, p-nitrobenzyloxycarbonyl; trialkylsilyl (e.g. trimethylsilyl and t-butyldimethylsilyl); alkylidene (e.g. methylidene); benzylidene and substituted benzylidene groups.

Methods appropriate for removal of hydroxy and amino protecting groups include, for example, nucleophilic displacement, acid-, base, metal- or enzymically-catalysed hydrolysis, or photolytically for groups such as o-nitrobenzyloxycarbonyl, or with fluoride ions for silyl groups. For example, methylether protecting groups for hydroxy groups may be removed by trimethylsilyliodide. A tert-butyl ether protecting group for a hydroxy group may be removed by hydrolysis, for example by use of hydrochloric acid in methanol.

Examples of protecting groups for amide groups include aralkoxymethyl (e.g. benzyloxymethyl and substituted benzyloxymethyl); alkoxymethyl (e.g. methoxymethyl and trimethylsilylethoxymethyl); tri alkyl/arylsilyl (e.g. trimethylsilyl, t-butyldimethylsily, t-butyldiphenylsilyl); tri alkyl/arylsilyloxymethyl (e.g. t-butyldimethylsilyloxymethyl, t-butyldiphenylsilyloxymethyl); 4-alkoxyphenyl (e.g. 4-methoxyphenyl); 2,4-di(alkoxy)phenyl (e.g. 2,4-dimethoxyphenyl); 4-alkoxybenzyl (e.g. 4-methoxybenzyl); 2,4-di(alkoxy)benzyl (e.g. 2,4-di(methoxy)benzyl); and alk-1-enyl (e.g. allyl, but-1-enyl and substituted vinyl e.g. 2-phenylvinyl).

Aralkoxymethyl groups may be introduced onto the amide group by reacting the latter group with the appropriate aralkoxymethyl chloride, and removed by catalytic hydrogenation. Alkoxymethyl, tri alkyl/arylsilyl and tri alkyl/silyloxymethyl groups may be introduced by reacting the amide with the appropriate chloride and removing with acid; or in the case of the silyl containing groups, fluoride ions. The alkoxyphenyl and alkoxybenzyl groups are conveniently introduced by arylation or alkylation with an appropriate halide and removed by oxidation with ceric ammonium nitrate. Finally alk-1-enyl groups may be introduced by reacting the amide with the appropriate aldehyde and removed with acid.

The following examples are for illustration purposes and are not intended to limit the scope of this application. Each exemplified compound represents a particular and independent aspect of the invention. In the following non-limiting Examples, unless otherwise stated:
(i) evaporations were carried out by rotary evaporation in vacuo and work-up procedures were carried out after removal of residual solids such as drying agents by filtration;
(ii) operations were carried out at room temperature, that is in the range 18-25° C. and under an atmosphere of an inert gas such as argon or nitrogen;
(iii) yields are given for illustration only and are not necessarily the maximum attainable;
(iv) the structures of the end-products of the Formula (I) were confirmed by nuclear (generally proton) magnetic resonance (NMR) with a field strength (for proton) of 300 MHz (generally using a Varian Gemini 2000) or 400 MHz (generally using a Bruker Avance DPX400), unless otherwise stated, and mass spectral techniques; proton magnetic resonance chemical shift values were measured on the delta scale and peak multiplicities are shown as follows: s, singlet; d, doublet; t, triplet; m, multiplet; br, broad; q, quartet, quip, quintet; any reference to DMSO as a NMR solvent should be taken to mean DMSO-d6;
(v) intermediates were not generally fully characterised and purity was assessed by thin layer chromatography (TLC), high-performance liquid chromatography (HPLC), infra-red (IR) or NMR analysis; and
(vi) Purification by chromatography generally refers to flash column chromatography, on silica unless otherwise stated. Column chromatography was generally carried out using prepacked silica cartridges (from 4 g up to 400 g) such as Redisep™ (available, for example, from Presearch Ltd, Hitchin, Herts, UK) or Biotage (Biotage UK Ltd, Hertford, Herts, UK), eluted using a pump and fraction collector system;
(vii) Mass spectra (MS) data was generated on an LCMS system where the HPLC component comprised generally either a Agilent 1100 or Waters Alliance HT (2790 & 2795) equipment and was run on a Phemonenex Gemini C18 5 μm, 50×2 mm column (or similar) eluting with either acidic eluent (for example, using a gradient between 0-95% water/acetonitrile with 5% of a 1% formic acid in 50:50 water:acetonitrile (v/v) mixture; or using an equivalent solvent system with methanol instead of acetonitrile), or basic eluent (for example, using a gradient between 0-95% water/acetonitrile with 5% of a 0.1% 880 Ammonia in acetonitrile mixture); and the MS component comprised generally a Waters ZQ spectrometer. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is (M+H)+ for positive ion mode and (M−H)⁻ for negative ion mode;
(viii) Suitable microwave reactors include “Smith Creator”, “CEM Explorer”, “Biotage Initiator sixty” and “Biotage Initiator eight”.
(ix) Preparative HPLC separations were run on standard Gilson™ HPLC equipment using a 150×21.2 mm Phenomenex Luna 10 micron C18(2) 100A column, and a standard gradient elution method (5-95% acetonitrile gradient with water as co-solvent and 0.2% trifluoroacetic acid as modifier, 12.5 min gradient with a 2.5 min hold at 95% acetonitrile) run on Unipoint software.

### ABBREVIATIONS

CDI Carbonyl di-imidazole

### DCM Dichloromethane

DIAD Diisopropyl azodicarboxylate
DMA Dimethyl acetamide
DMAP 4-dimethylamino pyridine
EDAC N-ethyl-N′-(3-dimethylaminopropyl)-carbodiimide
EtOAc Ethyl acetate

### NMP N-methylpyrrolidone

MgSO₄ magnesium sulfate
THF tetrahydrofuran
DMSO dimethylsulfoxide
MeOH methanol
EtOH ethanol
DMF dimethylformamide
MTBE methyl-tert-butylether
RT room temperature
All compound names were derived using a computer package such as ACD NAME.

### EXAMPLE 1

### 2-{3-(Benzyloxy)-5-[(1S)-2-methoxy-1-methylethoxy]phenyl}-3H-imidazo[4,5-b]pyridine

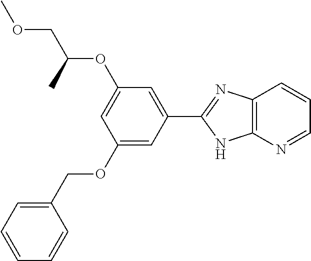

A solution of N-(2-aminopyridin-3-yl)-3-(benzyloxy)-5-[(1S)-2-methoxy-1-methylethoxy]benzamide in a mixture of xylene (27 mL) and glacial acetic acid (9 mL) was stirred at 140-150° C. under argon for 4.5 hrs. The solvent was evaporated to give a solid (˜1 g); this was chromatographed (40 g silica column, eluting with a gradient of DCM containing 0 to 4% MeOH) to give fractions which were combined and crystallised from EtOAC to give the title compound as a colourless amorphous solid (150 mg). ¹H NMR (300 MHz, DMSO) δ 1.20-1.30 (m, 3H), 3.29-3.32 (m, 10H), 3.41-3.59 (m, 2H), 4.64-4.77 (m, 1H), 5.13-5.24 (m, 2H), 6.67-6.78 (m, 1H), 7.18-7.27 (m, 2H), 7.28-7.44 (m, 8H), 7.45-7.53 (m, 6H), 7.82-8.12 (m, 1H), 8.19-8.47 (m, 1H), 12.86-13.60 (m, 1H); m/z 390 (M+H)⁺, 388 (M−H)⁻.

The requisite N-(2-aminopyridin-3-yl)-3-(benzyloxy)-5-[(1S)-2-methoxy-1-methylethoxy]benzamide starting material was prepared as follows:

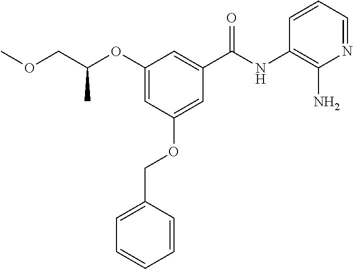

A stirred solution of 3-(benzyloxy)-5-[(1S)-2-methoxy-1-methylethoxy]benzoic acid (3.16 g, 10 mmol) and 2,3 diamino pyridine (1.09 g, 10 mmol) in DMF (20 ml) was treated with EDAC (2.4 g, 12.5 mmol), and the resulting solution stirred for 2 hrs. Water (˜100 ml) was added and the mixture extracted twice with EtOAc; the combined organic extracts were washed twice with water, once with saturated sodium bicarbonate solution and once with brine; the solution was dried (MgSO₄) and evaporated to give ˜3 g of a dark brown oil. This was chromatographed (Biotage 90 g silica column, eluting with gradient of DCM containing 0-5% MeOH) to give the title compound as a colourless foam (1.33 g), ¹H NMR (300 MHz, DMSO) δ 1.21 (d, 3H), 3.28 (s, 3H), 3.40-3.54 (m, 2H), 4.62-4.75 (m, 1H), 5.10-5.19 (m, 2H), 5.64-5.81 (m, 2H), 6.55-6.63 (m, 1H), 6.74-6.80 (m, 1H), 7.09-7.16 (m, 1H), 7.16-7.23 (m, 1H), 7.26-7.54 (m, 6H), 7.80-7.88 (m, 1H), 9.57 (s, 1H); m/z 408 (M+H)⁺, 406 (M−H)⁻.

The requisite 3-(benzyloxy)-5-[(1S)-2-methoxy-1-methylethoxy]benzoic acid was prepared as follows:

### Methyl 3-(benzyloxy)-5-hydroxybenzoate

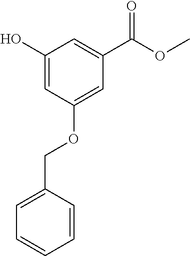

To a stirred solution of methyl 3,5-dihydroxybenzoate (5.95 mol) in DMF (6 L) was added potassium carbonate (9 mol), and the suspension stirred at ambient temperature under argon. To this was added benzyl bromide (8.42 mol) slowly over 1 hour, with a slight exotherm, and the reaction mixture stirred overnight at ambient temperature. The reaction was quenched cautiously with ammonium chloride solution (5 L) followed by water (35 L). The aqueous suspension was extracted with dichloromethane (1×3 L and 2×5 L). The combined extracts were washed with water (10 L) and dried overnight (MgSO₄). The solution was evaporated in vacuo, and the crude product chromatographed in 3 batches (flash column, 3×2 kg silica, eluting with a gradient consisting of hexane containing 10% dichloromethane, to neat dichloromethane, to dichloromethane containing 50% ethyl acetate) to eliminate starting material. The crude eluant was further chromatographed in 175 g batches (5 kg normal-phase silica, eluting with isohexane containing 20% v/v of ethyl acetate) to give the desired compound (21% yield). ¹H NMR δ (d₆-DMSO): 3.8 (s, 3H), 5.1 (s, 2H), 6.65 (m, 1H), 7.0 (m, 1H), 7.05 (m, 1H), 7.3-7.5 (m, 5H), 9.85 (br s, 1H)

### Methyl 3-(benzyloxy)-5-[(1S)-2-methoxy-1-methylethoxy]benzoate

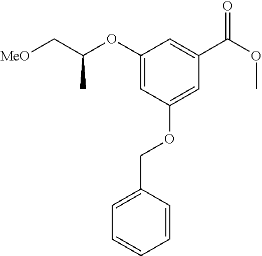

To a solution of methyl 3-(benzyloxy)-5-hydroxybenzoate (77.4 mmol) in THF was added polymer-supported triphenylphosphine (51.7 g of 3 mmol/g loading, 155 mmol) and (R)-(−)-1-methoxy-2-propanol (102 mmol). The stirred solution was blanketed with argon and cooled in an ice bath. A solution of diisopropylazodicarboxylate (116 mmol) was added dropwise by syringe over 10 minutes. The solution was stirred for 20 minutes and filtered, washing the residue with THF (500 ml). The filtrate and washings were combined, and evaporated to give the desired compound which was used without further purification.

¹H NMR δ (d₆-DMSO): 3.26 (s, 3H), 3.44 (m, 2H), 3.82 (s, 3H), 4.63 (m, 1H), 5.14 (s, 2H), 6.85 (s, 1H), 7.05 (s, 1H), 7.11 (s, 1H), 7.30-7.47 (m, 5H)

The ¹H NMR spectrum also contained signals consistent with a small amount of bis(1-methylethyl)hydrazine-1,2-dicarboxylate.

### 3-(Benzyloxy)-5-[(1S)-2-methoxy-1-methylethoxy]benzoic acid

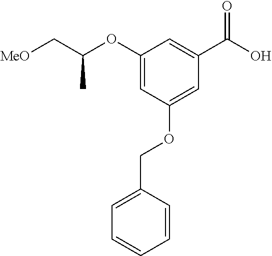

A solution of methyl 3-(benzyloxy)-5-[(1S)-2-methoxy-1-methylethoxy]benzoate (77.4 mmol) in a mixture of THF (232 ml) and methanol (232 ml) was treated with a solution of 2M sodium hydroxide (232 mmol), and the reaction mixture stirred for 4 hours at ambient temperature. The resulting solution was diluted with water (250 mL) and most of the organic solvent removed in vacuo. The resulting suspension was washed with diethyl ether (3×200 mL) and the organic washings discarded. The resulting aqueous solution was acidified to pH4 with 2M hydrochloric acid solution and extracted with ethyl acetate (2×200 mL). The extracts were combined, washed with brine, dried (MgSO₄), and evaporated to give the desired compound (99% yield). ¹H NMR δ (d₆-DMSO): 1.20 (d, 3H), 3.46 (m, 2H), 4.64 (m, 1H), 5.15 (s, 2H), 6.83 (app t, 1H), 7.06 (s, 1H), 7.13 (s, 1H), 7.30-7.49 (m, 5H), 12.67 (br s, 1H).

### EXAMPLE 2

### 8-{3-(Benzyloxy)-5-[(1S)-2-methoxy-1-methylethoxy]phenyl}-9H-purine

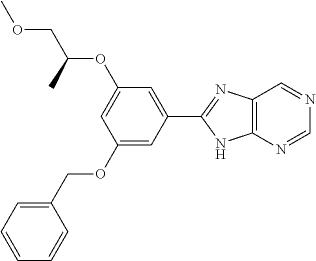

A solution of N-(4-aminopyrimidin-5-yl)-3-(benzyloxy)-5-[(1S)-2-methoxy-1-methylethoxy]benzamide (400 mg, 1 mmol) in butyronitrile (8 ml) was treated with glacial acetic acid (8 drops, catalytic) was heated at 200° C. for 2 hours in a Biotage “Initiator”™ Microwave. The reaction mixture was cooled and the resulting precipitate filtered off, washed sequentially with acetonitrile and ether, and dried to give the title compound as a colourless solid (300 mg). ¹H NMR (400 MHz, DMSO) δ 1.23-1.31 (m, 3H), 3.46-3.58 (m, 2H), 4.74 (dq, 1H), 5.22 (s, 2H), 6.81 (s, 1H), 7.32-7.57 (m, 7H), 8.91 (s, 1H), 9.11 (s, 1H), 13.83 (s, 1H); m/z 389 (M−H)⁻.

### N-(4-aminopyrimidin-5-yl)-3-(benzyloxy)-5-[(1S)-2-methoxy-1-methylethoxy]benzamide

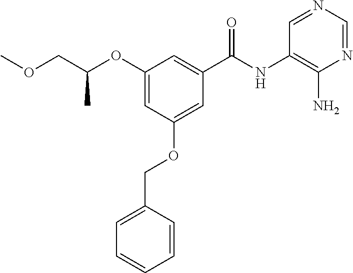

The requisite N-(4-aminopyrimidin-5-yl)-3-(benzyloxy)-5-[(1S)-2-methoxy-1-methylethoxy]benzamide starting material was made by a method essentially similar to that described in Example 1, starting from 3-(benzyloxy)-5-[(1S)-2-methoxy-1-methylethoxy]benzoic acid and 4,5-diaminopyrimidine, ¹H NMR (300 MHz, DMSO) δ 1.18-1.28 (m, 3H), 3.40-3.55 (m, 2H), 4.69 (dt, 1H), 5.15 (s, 2H), 5.74 (d), 6.70-6.90 (m, 3H), 7.12-7.25 (m, 2H), 7.28-7.51 (m, 5H), 8.14 (s, 1H), 8.26 (s, 1H), 9.59 (s, 1H); m/z 409 (M+H)⁺407 (M−H)⁻.

### EXAMPLE 3

### 6-Chloro-2-{3-[(1S)-2-methoxy-1-methylethoxy]-5-[4-(methylsulfonyl)phenoxy]phenyl}-3H-imidazo[4,5-b]pyridine

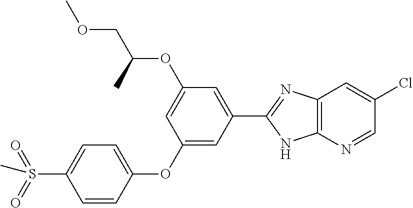

A solution of 3-[(1S)-2-methoxy-1-methylethoxy]-5-[4-(methylsulfonyl)phenoxy]benzoic acid (190 mg, 0.5 mmol) in butyronitrile (4 ml) was treated with CDI (97 mg, 0.60 mmol) and stirred for 15 min. 2,3-Diamino-5-chloropyridine (72 mg, 0.5 mmol) was added and the reaction mixture heated in a microwave (CEM DISCOVER 300W™) at 180° C. for 20 mins. The reaction mixture was purified by chromatography (40 g silica column, eluting with a gradient of EtOAc containing 0-10% methanol over 9 mins to give the title compound (91 mg, 37%) as a pink solid. ¹H NMR (400 MHz, DMSO) δ 1.30 (3H, d), 3.20 (3H, s), 3.35 (3H, s), 3.49-3.61 (2H, m), 4.66-4.77 (1H, m), 6.92 (1H, s), 7.28 (2H, d), 7.56 (1H, s), 7.72 (1H, s), 7.95 (2H, d), 8.11 (1H, s), 8.36 (1H, s), 13.62 (1H, s), M/z: 488, 490 (M+H)⁺.

The following compounds were prepared by the method described for Example 3, starting from the appropriate benzoic acid and diamino heterocycle:

### EXAMPLE 4

8-{3-[(1S)-2-Methoxy-1-methylethoxy]-5-[4-(methylsulfonyl)phenoxy]phenyl}-9H-purine

### EXAMPLE 5

2-{3-Isopropoxy-5-[(1S)-1-methyl-2-phenylethoxy]phenyl}-3H-imidazo[4,5-b]pyridine

### EXAMPLE 6

6-Bromo-2-{3-[(1S)-2-methoxy-1-methylethoxy]-5-[4-(methylsulfonyl)phenoxy]phenyl}-3H-imidazo[4,5-b]pyridine

### EXAMPLE 7

5-Bromo-2-{3-[(1S)-2-methoxy-1-methylethoxy]-5-[4-(methylsulfonyl)phenoxy]phenyl}-1H-imidazo[4,5-b]pyrazine

### EXAMPLE 8

2-{3-(Benzyloxy)-5-[(1S)-2-methoxy-1-methylethoxy]phenyl}-5-bromo-1H-imidazo[4,5-b]pyrazine

| Ex No | Structure | NMR | M/z |
|---|---|---|---|
| 4 | 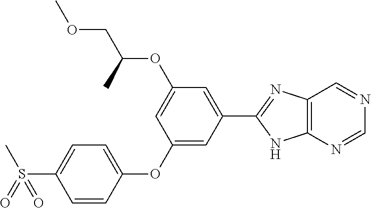 | ¹H NMR (400 MHz, DMSO) δ 1.31 (3H, d), 3.19 (3H, s), 3.35 (3H, s), 3.49-3.61 (2H, m), 4.69-4.78 (1H, m), 6.91-6.95 (1H, m), 7.29 (2H, d), 7.55-7.58 (1H, m), 7.72-7.75 (1H, m), 7.96 (2H, d), 8.90 (1H, s), 9.02 (1H, s), 13.70 (1H, s) | 455 (M + H)⁺ |
| 5 | 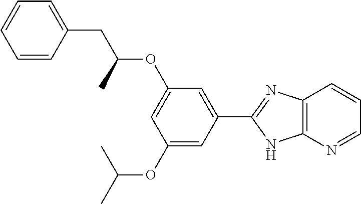 | ¹H NMR (400 MHz, DMSO) δ 1.27-1.36 (9H, m), 2.88-2.97 (1H, m), 3.00-3.09 (1H, m), 4.60-4.71 (1H, m) , 4.74-4.84 (1H, m), 6.52 (1H, s), 7.17-7.24 (2H, m), 7.26-7.33 (4H, m), 7.35-7.43 (2H, m), 7.92 (1H, d), 8.33 (1H, d), 12.90 (1H, d) | 388 (M + H)⁺ |
| 6 | 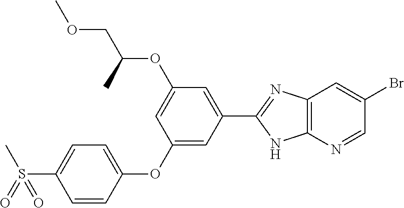 | ¹H NMR (400 MHz, DMSO) δ 1.31 (3H, d), 3.20 (3H, s), 3.35 (3H, s), 3.48-3.61 (2H, m), 4.68-4.77 (1H, m), 6.94 (1H, s), 7.28 (2H, d), 7.57 (1H, s), 7.73 (1H, s), 7.96 (2H, d), 8.27 (1H, s), 8.45 (1H, s), 13.71 (1H, s) | 532, 534 (M + H)⁺ |
| 7 | 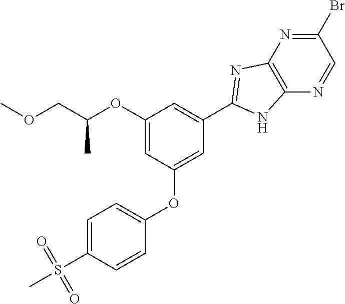 | ¹H NMR (400 MHz, DMSO) δ 1.30 (d, 3H), 3.48-3.60 (m, 2H), 4.74-4.84 (m, 1H), 7.00- 7.05 (m, 1H), 7.28-7.36 (m, 2H), 7.56 (s, 1H), 7.74 (s, 1H), 7.93-8.02 (m, 2H), 8.55 (s, 1H), 14.16 (s, 1H) | 531, 533 (M − H)⁻ |
| 8 | 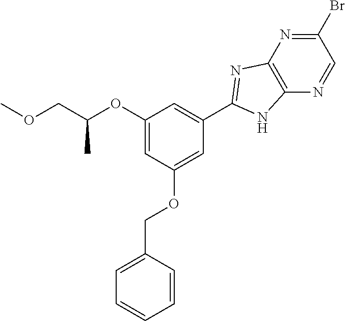 | ¹H NMR (400 MHz, DMSO) δ 1.27 (d, 3H), 3.35 (s, 3H), 3.47-3.58 (m, 2H), 4.69-4.79 (m, 1H), 5.22 (s, 2H), 6.82-6.85 (m, 1H), 7.32-7.56 (m, 7H), 8.54 (s, 1H) | 469, 471 (M + H)⁺ |

NOTE: Examples 7 and 8: The examples prepared from 2,3 diamino 5-bromo pyrazine required 2 hrs microwave heating instead of 15 mins; the requisite 2,3 diamino 5-bromo pyrazine may be prepared as described in: Bioorganic & Medicinal Chemistry 7 (1999) 1059,
New Imidazo[1,2-a]pyrazine Derivatives with Bronchodilatory and Cyclic Nucleotide Phosphodiesterase Inhibitory Activities (Olivier Vitse, Florence Laurent, Tristan M. Pocock, Veronique Benezech, Lahcen Zanik, Keith R. F. Elliott, Guy Subra, Marine Portet, Jacques Bompart, Jean-Pierre Chapat, Roger C. Small, Alain Michel and Pierre-Antoine Bonneta).

### EXAMPLE 4

The requisite 3-[(1S)-2-methoxy-1-methylethoxy]-5-[4-(methylsulfonyl)phenoxy]benzoic acid starting material was prepared as follows:

### Methyl 3-hydroxy-5-[(1S)-2-methoxy-1-methylethoxy]benzoate

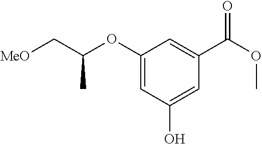

Methyl 3-(benzyloxy)-5-[(1S)-2-methoxy-1-methylethoxy]benzoate (50.0 g; 0.152 mmol) was dissolved in a mixture of THF:ethanol (600 ml) and the flask evacuated and purged with nitrogen (3 times). 10% Palladium on carbon (5.0 g) was added and the flask further evacuated and finally purged with hydrogen gas. The reaction mixture was stirred at ambient temperature for 20 hours until completion. The reaction mixture was evacuated and purged with nitrogen (3 times). The catalyst was filtered off, and the filtrate concentrated in vacuo to give the desired compound (36.7 g), ¹H NMR δ (d₆-DMSO): 1.2 (d, 3H), 3.25 (s, 3H), 3.44 (m, 2H), 3.82 (s, 3H), 4.55 (m, 1H), 6.6 (s, 1H), 6.9 (s, 1H), 6.95 (s, 1H), 9.8 (s, 1H).

### Methyl 3-[(1S)-2-methoxy-1-methylethoxy]-5-[4-(methylsulfonyl)phenoxy]benzoate

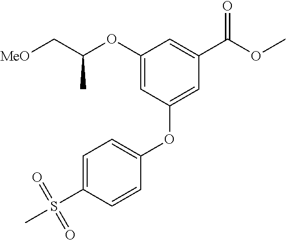

A suspension of methyl 3-hydroxy-5-[(1S)-2-methoxy-(1-methylethyl)oxy]benzoate (154 mmol), boronic acid (1.1 equivalents), copper (II) acetate (1.1 equivalents), triethylamine (5 equivalents) and freshly activated 4 Å molecular sieves (200 g) in DCM (500 ml) was stirred at ambient temperature and under ambient atmosphere for 2 days. The reaction mixture was filtered, the DCM removed in vacuo and the residual oil partitioned between ethyl acetate and 1-2M hydrochloric acid. The ethyl acetate layer was separated, washed with aqueous sodium hydrogen carbonate and brine, dried (MgSO₄), and evaporated to a residue which was chromatographed on silica (with 20-60% ethyl acetate in isohexane as eluant) to give the desired ester (58% yield), ¹H NMR δ (d₆-DMSO): 1.2 (d, 3H), 3.2 (s, 3H), 3.26 (s, 3H), 3.44 (m, 2H), 3.8 (s, 3H), 4.65 (m, 1H), 7.05 (s, 1H), 7.11 (s, 1H), 7.2 (d, 2H), 7.3 (s, 1H), 7.9 (d, 2H)

### 3-[(1S)-2-Methoxy-1-methylethoxy]-5-[4-(methylsulfonyl)phenoxy]benzoic acid

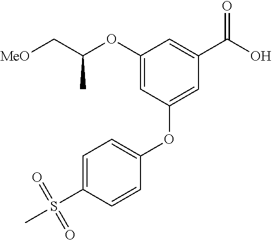

A solution of Methyl 3-[(1S)-2-methoxy-1-methylethoxy]-5-[4-(methylsulfonyl)phenoxy]benzoate (60.9 mmol) in THF (400 mlL) was treated with a solution of 1M sodium hydroxide (125 mmol), and the reaction mixture stirred for 13 hours at ambient temperature. Most of the organic solvent was removed in vacuo, and the remaining solution was diluted with water (150 ml). The resulting aqueous solution was acidified to pH4 with 1M citric acid solution, and extracted with ethyl acetate (2×100 ml). The extracts were combined, washed with brine, dried (MgSO₄), and evaporated to give the desired compound (83% yield).

¹H NMR δ (d₆-DMSO): 1.2 (d, 3H), 3.2 (s, 3H), 3.26 (s, 3H), 3.44 (m, 2H), 4.63 (m, 1H), 7.05 (s, 1H), 7.11 (s, 1H), 7.2 (d, 2H), 7.3 (s, 1H), 7.9 (d, 2H). m/z 479 (M−H)⁻

### EXAMPLE 5

The requisite 3-isopropoxy-5-[(1S)-1-methyl-2-phenylethoxy]benzoic acid starting material was prepared as follows:

### Methyl 3-isopropoxy-5-[(1S)-1-methyl-2-phenylethoxy]benzoate

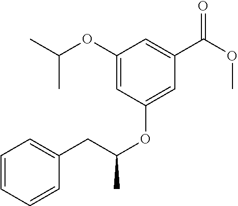

A solution of methyl 3-hydroxy-5-isopropoxybenzoate [CAS No. 752242-26-5; WO 2004/076420] (5 g, 23.8 mmol), triphenyl phosphine (9.35 g, 35.7 mmol) and (2R)-1-phenylpropan-2-ol (5 ml, 35.7 mmol) in DCM (150 ml) was cooled to 0° C. and stirred at this temperature for 30 mins. DIAD (7.02 ml, 35.7 mmol) was added dropwise over 20 mins and the reaction solution then stirred for a further 30 mins at 0° C. and then at ambient temperature overnight. Starting phenol was still present so the solution was recooled to 0° C. and extra triphenyl phosphine (4.7 g, 17.9 mmol) and (2R)-1-phenylpropan-2-ol (2.5 ml, 17.9 mmol) were added, followed by DIAD (2.5 ml, 17.9 mmol) added over 20 mins.

The reaction was stirred for a further 30 mins at 0° C. and then at ambient temperature for 2 hrs. The solvent was evaporated and the residue treated with a mixture of isohexane and diethyl ether (200 ml of 9:1 v/v); the resulting solid precipitate was filtered off and discarded and the filtrate evaporated. The residue was chromatographed on silica eluting with isohexane containing 10% v/v of EtOAc to give the title compound as a golden oil, (7.56 g, 97%), ¹H NMR δ (CDCl₃): 1.3-1.4 (m, 9H), 2.8-2.9 (dd, 1H), 3.0-3.1 (dd, 1H), 3.9 (s, 4.5-4.7 (m, 2H), 6.6 (s, 1H), 7.1-7.4 (m, 7H), m/z 329 (M+H)⁺

### 3-Isopropoxy-5-[(1S)-1-methyl-2-phenylethoxy]benzoic acid

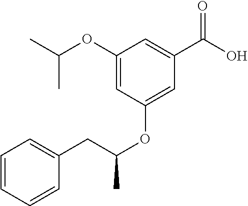

A solution of methyl 3-isopropoxy-5-[(1S)-1-methyl-2-phenylethoxy]benzoate (7.56 g, 23.05 mmol) in methanol (200 ml) was treated with aqueous sodium hydroxide solution (46.5 ml of 1M, 46.5 mmol), and the reaction mixture stirred at ambient temperature overnight. The resulting solution was acidified with 1M hydrochloric acid and extracted with EtOAc (3×150 ml). The combined extracts were dried (MgSO₄) and evaporated to give the title compound as an oil which crystallised on standing (7.0 g, 97%), ¹H NMR δ (CDCl₃): 1.3-1.4 (m, 9H), 2.8-2.9 (dd, 1H), 3.05-3.15 (dd, 1H), 4.5-4.7 (m, 2H), 6.6 (s, 1H), 7.1-7.4 (m, 7H), m/z 315 (M+H)⁺

### EXAMPLE 9

### 2-{3-[(1S)-2-Methoxy-1-methylethoxy]-5-[4-(methylsulfonyl)phenoxy]phenyl}-3H-imidazo[4,5-b]pyridine

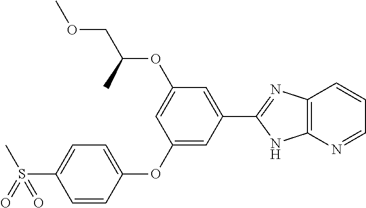

A solution of 3-(3H-imidazo[4,5-b]pyridin-2-yl)-5-[(1S)-2-methoxy-1-methylethoxy]phenol (100 mg, 0.33 mmol) and 4-fluorophenyl sulfone (64 mg, 0.37 mmol) in NMP was treated with potassium carbonate (115 mg, 0.84 mmol) and the resulting suspension heated at 115° C. overnight. The reaction mixture was poured into water and extracted with DCM; drying (Phase-separating cartridge) and high vacuum evaporation yielded a brown oil. This was chromatographed (Optix-10™, 12 g silica column, gradient elution with DCM containing 0-7% methanol). The product was contaminated with NMP and so was taken up in dichloromethane and washed with water (×5), passed through a phase separator and concentrated in vacuo. NMR indicated that NMP was still present, although in reduced quantities; the product was taken up in ethyl acetate and washed with water a further five times, the organics dried over magnesium sulfate and concentrated in vacuo to afford the pure title compound (63 mg). ¹H NMR (400 MHz, CDCl₃) δ 1.38 (d, 1H), 3.10 (s, 3H), 3.47 (s, 3H), 3.59 (d, 2H), 4.70 (dd, 1H), 6.88 (s, 1H), 7.21 (m, 3H), 7.64 (s, 1H), 7.79 (s, 1H), 7.91 (d, 2H), 8.14 (d, 1H), 8.44 (s, 1H), 14.43 (s, 1H); m/z 454 (M+H)⁺.

The requisite 3-(3H-imidazo[4,5-b]pyridin-2-yl)-5-[(1S)-2-methoxy-1-methylethoxy]phenol starting material was prepared as follows:

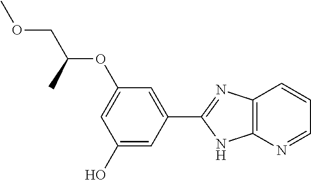

A solution of 2-{3-(benzyloxy)-5-[(1S)-2-methoxy-1-methylethoxy]phenyl}-3H-imidazo[4,5-b]pyridine (Example 1, 5.2 g, 13.4 mmol) in a mixture of MeOH and EtOAc (500 ml, 1:1) was purged with argon; catalyst (600 mg of 10% palladium-on-charcoal) was added, and the resulting suspension was stirred in an atmosphere of hydrogen for 16 hours. TLC indicated that all starting material was consumed. The catalyst was filtered off using a Glass Fibre paper and washed through with more 1:1 MeOH:EtOAc. The filtrate and washings were evaporated to give the title compound as a colourless solid (4 g). ¹H NMR (300 MHz, DMSO) δ 1.25 (d, 3H), 3.30 (s, 3H), 3.42-3.56 (m, 2H), 4.55-4.69 (m, 1H), 6.43-6.49 (m, 1H), 7.16-7.28 (m, 3H), 7.90-8.01 (m, 1H), 8.26-8.34 (m, 1H), 8.58-10.98 (m, HA 11.99-13.96 (m, 1H); m/z 300 (M+H)⁺, 298 N—H)⁻.

### EXAMPLES 10-18

The following compounds were prepared using a method essentially similar to that described in Example 9:

### EXAMPLE 10

2-{3-[4-(Azetidin-1-ylcarbonyl)-2-fluorophenoxy]-5-[(1S)-2-methoxy-1-methylethoxy]phenyl}-3H-imidazo[4,5-b]pyridine

### EXAMPLE 11

3-Chloro-4-{3-(3H-imidazo[4,5-b]pyridin-2-yl)-5-[(1S)-2-methoxy-1-methylethoxy]phenoxy}-N,N-dimethylbenzamide

### EXAMPLE 12

2-{3-[2-Fluoro-4-(pyrrolidin-1-ylcarbonyl)phenoxy]-5-[(1S)-2-methoxy-1-methylethoxy]phenyl}-3H-imidazo[4,5-b]pyridine

### EXAMPLE 13

2-{3-[2-Chloro-4-(pyrrolidin-1-ylcarbonyl)phenoxy]-5-[(1S)-2-methoxy-1-methylethoxy]phenyl}-3,1-imidazo[4,5-b]pyridine

### EXAMPLE 14

2-{3-[2-Fluoro-4-(methylsulfonyl)phenoxy]-5-[(1S)-2-methoxy-1-methylethoxy]phenyl}-3H-imidazo[4,5-b]pyridine

### EXAMPLE 15

3-Fluoro-4-{3-(3H-imidazo[4,5-b]pyridin-2-yl)-5-[(1S)-2-methoxy-1-methylethoxy]phenoxy}-N,N-dimethylbenzamide

### EXAMPLE 16

4-{3-(3H-Imidazo[4,5-b]pyridin-2-yl)-5-[(1S)-2-methoxy-1-methylethoxy]phenoxy}benzonitrile

### EXAMPLE 17

2-{3-(3H-Imidazo[4,5-b]pyridin-2-yl)-5-[(1S)-2-methoxy-1-methylethoxy]phenoxy}benzonitrile

### EXAMPLE 18

2-{3-Isopropoxy-5-[4-(methylsulfonyl)phenoxy]phenyl}-3H-imidazo[4,5-b]pyridine

| Ex No | Structure | NMR | M/z |
|---|---|---|---|
| 10 | 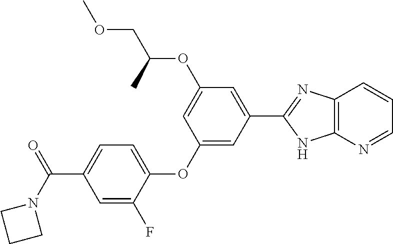 | ¹H NMR (400 MHz, CDCl₃) δ 1.35 (d, 3H), 2.36 (quintet, 2H), 3.44 (s, 3H), 3.53 (dd, 1H), 3.61 (dd, 1H), 4.28 (d, 4H), 4.73 (s, 1H), 6.80 (s, 1H), 7.11 (t, 1H), 7.29 (s, 1H), 7.37 (d, 1H), 7.54 (s, 2H), 7.69 (s, 1H), 8.16 (s, 1H), 8.46 (d, 1H), 14.12 (s, 1H) | 477 (M + H)⁺ |
| 11 | 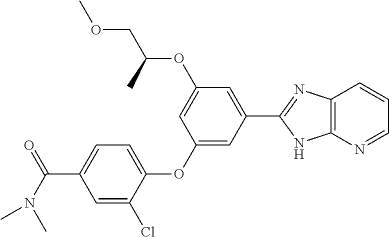 | ¹H NMR (300 MHz, CDCl₃) δ 1.35 (d, 3H), 3.19 (bs, 6H), 3.44 (s, 3H), 3.71 (s, 2H), 4.76 (m, 1H), 6.79 (s,1H), 7.05 (d, 1H), 7.31 (s, 2H), 7.53 (s, 1H), 7.60 (s, 1H), 7.72 (s, 1H), 8.20 (bs, 1H), 8.50 (d, 1H), 14.27 (bs, 1H) | 481 (M + H)⁺ |
| 12 | 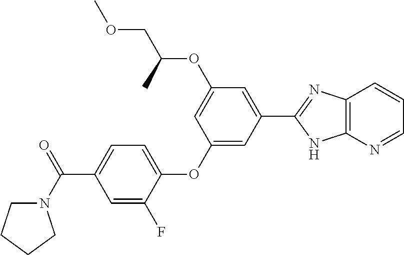 | | 491 (M + H)⁺ |
| 13 | 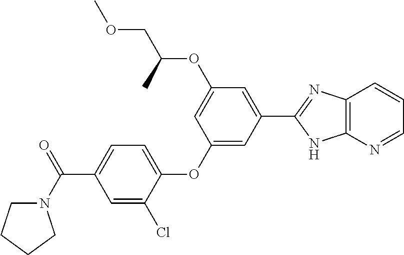 | ¹HNMR (400 MHz, CDCl₃) δ 1.34 (d, 1H), 2.00 (m, 4H), 3.43 (s, 3H), 3.54 (t, 2H), 3.64 (s, 2H), 3.71 (t, 2H), 4.74 (dq, 1H), 6.72 (t, 1H), 7.00 (s, 1H), 7.02 (s, 1H), 7.37 (s, 1H), 7.41 (dd, 1H), 7.49 (dd, 1H), 7.61 (s, 1H), 7.67 (d, 1H), 8.41 (d, 1H), 8.48 (d, 1H) | 507 (M + H)⁺ |
| 14 | 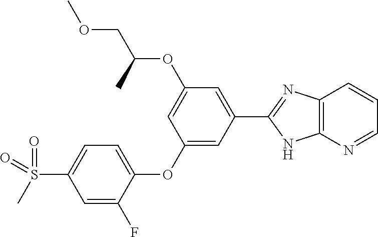 | ¹H NMR (400 MHz, CDCl₃) δ 1.39 (d, 3H), 3.15 (s, 3H), 3.50 (s, 3H), 3.70 (m, 2H), 4.76 (dq, 1H), 6.80 (t, 1H), 7.26 (d, 2H), 7.43 (s, 1H), 7.53 (dd, 2H), 7.73 (d, 2H), 7.81 | 472 (M + H)⁺ |
| 15 | 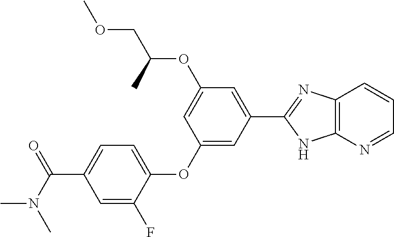 | ¹H NMR (400 MHz, CDCl₃) δ 1.27 (d, 3H), 3.04 (d, 6H), 3.37 (s, 3H), 3.57 (s, 2H), 4.64 (dd, 1H), 6.68 (s, 1H), 7.03 (t, 1H), 7.14 (d, 2H), 7.23 (d, 1H), 7.39 (dd, 1H), 7.49 (s, 1H), 8,24 (d, 1H), 8.39 (d, 2H) | 465 (M + H)⁺ |
| 16 | 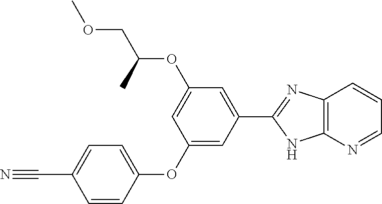 | ¹H NMR (400 MHz, CDCl₃) δ 1.47 (s, 3H), 3.50 (s, 3H), 3.62 (m, 2H), 4.73 (dq, 1H), 6.77 (t, 1H), 7.58 (s, 1H), 7.42 (s, 1H), 7.09 (d, 2H), 7.49 (dd, 1H), 7.67 (d, 2H), 8.35 (d,1H), 8.50 (d, 1H), 11.80 (s, 1H). | 401 (M + H)⁺ |
| 17 | 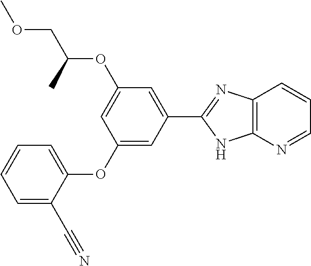 | ¹H NMR (400 MHz, CDCl₃) δ 1.40 (d, 3H) 3.50 (s, 3H), 3.64 (dd, 2H), 4.86 (s,1H), 6.92 (s,1H), 7.12 (d, 1H), 7.32 (m, 4H), 7.51 (s, 1H), 7.60 (m, 2H), 7.75 (d,1H). | 401 (M + H)⁺ |
| 18 | 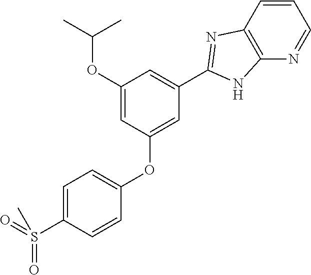 | ¹H NMR (400 MHz, DMSO) δ 1.34 (d, 6H), 3.26 (s, 3H), 4.77 (quintet, 1H), 6.92 (t, 1H), 7.27-7.37 (m, 3H), 7.51-7.55 (m, 1H), 7.66-7.72 (m, 1H), 7.97 (d, 2H), 8.07- 8.14 (m, 1H), 8.38-8.45 (m, 1H) | 424 (M + H)⁺ |

### EXAMPLE 18

This Example was prepared at 200° C. in the microwave (Biotage Initiator EXP60™) using butyronitrile as solvent, and starting from 3-(3H-imidazo[4,5-b]pyridin-2-yl)-5-isopropoxyphenol, the requisite starting material being prepared as follows:

### Methyl 3-(benzyloxy)-5-isopropoxybenzoate

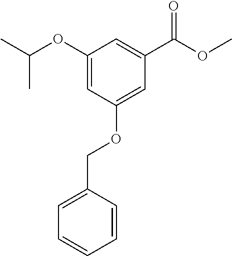

To a solution of methyl 3-hydroxy-5-isopropoxybenzoate (see Example 5) (25 g) in DMF (250 ml) was added anhydrous potassium carbonate (297 mmol), and benzyl bromide (143 mmol). The mixture was stirred at 60° C. for 5 hours, then cooled to room temperature. The solvent was removed in vacuo and the residue partitioned between ethyl acetate and water. The organics were combined and washed with further water, brine, dried (MgSO₄) and concentrated in vacuo to give the desired compound (37 g) which was used without further purification, ¹H NMR δ (d₆-DMSO): 1.26 (d, 6H), 3.84 (s, 3H), 4.61-4.70 (m, 1H), 5.12 (s, 2H), 6.84 (t, 1H), 7.05 (t, 1H), 7.12-7.15 (m, 1H), 7.31-7.37 (m, 1H), 7.40 (t, 2H), 7.46 (d, 2H).

### 3-(Benzyloxy)-5-isopropoxybenzoic acid

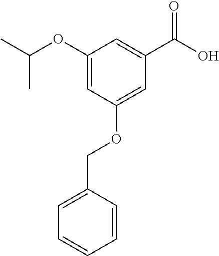

To a solution of methyl 3-(benzyloxy)-5-isopropoxybenzoate (37 g) in a 1:1 mixture of THF:methanol (300 ml) was added 4M sodium hydroxide solution (150 ml). The mixture was refluxed for 45 minutes, following which the organics were removed in vacuo. The aqueous was acidified to pH4 with hydrochloric acid (2M), and extracted with ethyl acetate. The organics were combined, washed with water and brine, dried (MgSO₄) and concentrated in vacuo to give the desired compound (33.45 g), which was used without further purification. ¹H NMR δ (d₆-DMSO): 1.26 (d, 6H), 4.59-4.69 (m, 1H), 5.15 (s, 2H), 6.80 (t, 1H), 7.04 (m, 1H), 7.12 (m, 1H), 7.33 (app t, 1H), 7.40 (t, 2H), 7.46 (d, 2H), 12.95 (s, 1H).

### N-(2-Aminopyridin-3-yl)-3-(benzyloxy)-5-isopropoxybenzamide

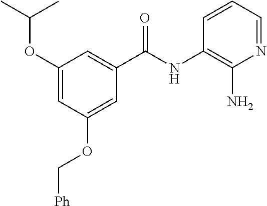

A solution of 3-(benzyloxy)-5-isopropoxybenzoic acid (10.0 g, 34.9 mmol) in anhydrous DMF (70 ml) was treated sequentially with 2,3-diaminopyridine (4.96 g, 45.4 mmol), DMAP (6.40 g, 52.4 mmol) and EDAC methiodide (15.57 g, 52.4 mmol). The brown solution was stirred at ambient temperature overnight and then concentrated to a dark brown oil, which was diluted with EtOAc, washed with sequentially with water (2×), brine, dried (MgSO₄) and concentrated to give a brown gummy solid (18.36 g). This was dissolved in EtOAc and precipitated with a few drops of hexane; the mixture was left to stand overnight and the resulting solid filtered off and washed with hexane to give the title compound as pale brown crystals (4.63 g, 35%). ¹H NMR (400 MHz, DMSO) δ 1.24-1.33 (m, 6H), 4.70 (quintet, 1H), 5.22 (s, 2H), 5.81 (s, 2H), 6.62 (dd, 1H), 6.76 (t, 1H), 7.16 (s, 1H), 7.24 (s, 1H), 7.30-7.38 (m, 1H), 7.41 (t, 2H), 7.47 (d, 2H), 7.54 (d, 1H), 7.82-7.91 (m, 1H), 9.62 (s, 1H); m/z 378 (M+H)⁺

### 2-[3-(Benzyloxy)-5-isopropoxyphenyl]-3H-imidazo[4,5-b]pyridine

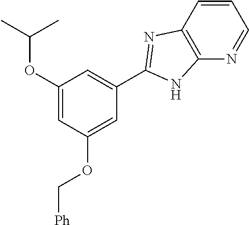

A suspension of N-(2-aminopyridin-3-yl)-3-(benzyloxy)-5-isopropoxybenzamide (2.20 g, 5.83 mmol) in butyronitrile (20 ml) was treated with 7 drops of glacial acetic acid, and the mixture heated in the microwave (Biotage Initiator EXP60™) for 2 hours at 200° C. The reaction mixture was cooled and left to stand at room temperature overnight; the crystalline material was filtered off, washed with cold butyronitrile and dried to give the title compound as an off-white solid (1.88 g, 90%). ¹H NMR (400 MHz, DMSO) δ1.32 (d, 6H), 4.72 (quintet, 1H), 5.25 (s, 2H), 6.67-6.75 (m, 1H), 7.25 (dd, 1H), 7.35 (t, 1H), 7.43 (t, 3H), 7.50 (d, 3H), 8.01 (d, 1H), 8.35 (d, 1H), 13.56 (s, 1H), m/z 360 (M+H)⁺

### 3-(3H-Imidazo[4,5-b]pyridin-2-yl)-5-isopropoxyphenol

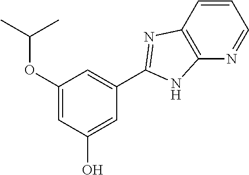

A solution of (2-[3-(benzyloxy)-5-isopropoxyphenyl]-3H-imidazo[4,5-b]pyridine (6.04 g, 16.8 mmol) in EtOAc (300 ml) and MeOH (300 ml) was treated with 10% Palladium on carbon (725 mg), and the resulting suspension stirred vigorously overnight in an atmosphere of hydrogen. The catalyst was filtered off and replaced with a similar amount of fresh catalyst; the suspension was then stirred under hydrogen over the weekend. The product was observed to have come out of solution, giving a grey suspension. The solids were filtered off and the product isolated from the catalyst by slurrying sequentially with methanol, hot ethyl acetate and DMF. The combined filtrates were concentrated to give the title compound as a pale yellow solid (842 mg, 19%). ¹H NMR (400 MHz, DMSO) δ 1.39 (s, 6H), 4.65 (quintet, 1H), 6.40-6.53 (m, 1H), 7.16-7.30 (m, 3H), 7.85-8.10 (m, 1H), 8.25-8.43 (m, 1H), 9.50 (s, 1H), 12.99-13.62 (m, 1H), m/z 270 (M+H)⁺

### EXAMPLE 19

### 3-{3-(3H-Imidazo[4,5-b]pyridin-2-yl)-5-[(1S)-2-methoxy-1-methylethoxy]phenoxy}-N,N-dimethylbenzamide

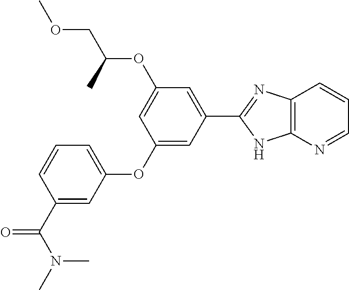

A solution of 3-(3H-imidazo[4,5-b]pyridin-2-yl)-5-[(1S)-2-methoxy-1-methylethoxy]phenol (see Example 9) (100 mg, 0.33 mmol) and 3-iodo-N,N-dimethylbenzamide (184 mg, 0.67 mmol) in DMA (3 ml) was treated with caesium carbonate (272 mg, 0.84 mmol) and bromotris(triphenylphosphine) copper (I) (102 mg, 0.26 mmol) [Synthetic Communications, 31(18), 2865-2879 (2001)], and the resulting suspension heated in a Biotage “Initiator”™ microwave at 150° C. for 2 hrs. The crude reaction mixture was concentrated in vacuo and the residue partitioned between ethyl acetate and water. The organic layer was washed with water (×3) and brine (×2), dried (MgSO₄) and evaporated. The crude material was purified by preparative HPLC to give the title compound. ¹H NMR (400 MHz, CDCl₃) δ 8.78 (s, 1H), 8.38 (d, 1H), 7.62 (s, 1H), 7.52 (m, 2H), 7.23 (s, 1H), 7.19 (d, 1H), 7.14 (s, 1H), 7.12 (s, 1H), 6.79 (s, 1H), 4.77 (m, 1H), 3.67 (m, 2H), 3.47 (s, 3H), 3.21 (s, 3H), 3.11 (s, 3H), 1.36 (d, 3H); m/z: 447 (M+H)⁺.

### EXAMPLE 20

### 2-{3-[(1S)-2-Methoxy-1-methylethoxy]-5-[3-(methylsulfinyl)phenoxy]phenyl}-3H-imidazo[4,5-b]pyridine

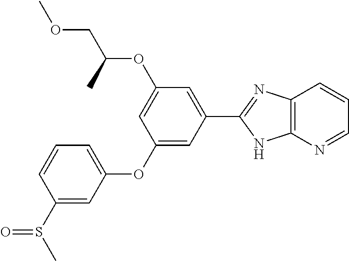

The title compound was prepared by a method essentially similar to that described in Example 19, starting from 1-bromo-3-(methylsulfinyl)benzene; m/z: 438 (M+H)⁺.

### EXAMPLES 21-28

The following Examples were prepared by an alkylation method essentially similar to that described in WO 2004/016611, [Johansson, Henrik; Lawitz, Karolina; Nikitidis, Grigorios; Sjoe, Peter; Storm, Peter; Preparation of imidazopyridines as Itk kinase inhibitors for use against asthma and allergic rhinitis], Examples 41-45, pp 59-60, starting from 3-(3H-imidazo[4,5-b]pyridin-2-yl)-5-[(1S)-2-methoxy-1-methylethoxy]phenol (see Example 9) and the appropriate benzyl or alkyl halide:

### EXAMPLE 21

4-({3-(3H-Imidazo[4,5-b]pyridin-2-yl)-5-[(1S)-2-methoxy-1-methylethoxy]phenoxy}methyl)benzonitrile.

### EXAMPLE 22

2-({3-(3H-Imidazo[4,5-b]pyridin-2-yl)-5-[(1S)-2-methoxy-1-methylethoxy]phenoxy}methyl)benzonitrile

### EXAMPLE 23

2-{3-[(3-Methoxybenzyl)oxy]-5-[(1S)-2-methoxy-1-methylethoxy]phenyl}-3H-imidazo[4,5-b]pyridine

### EXAMPLE 24

2-{3-[(2-Fluorobenzyl)oxy]-5-[(1S)-2-methoxy-1-methylethoxy]phenyl}-3H-imidazo[4,5-b]pyridine

### EXAMPLE 25

2-(3-[(1S)-2-Methoxy-1-methylethoxy]-5-{[4-(methylsulfonyl)benzyl]oxy}phenyl)-3H-imidazo[4,5-b]pyridine

### EXAMPLE 26

4-({3-(3H-Imidazo[4,5-b]pyridin-2-yl)-5-[(1S)-2-methoxy-1-methylethoxy]phenoxy}methyl)-N,N-dimethylbenzamide

| Ex No | Structure | NMR | M/z |
|---|---|---|---|
| 21 | 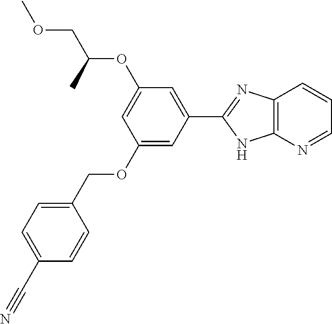 | ¹H NMR (400 MHz, DMSO) δ 1.26 (d, 3H), 3.30 (s, 3H), 3.44-3.57 (m, 2H), 4.67-4.78 (m, 1H), 5.32 (s, 2H), 6.75 (s, 1H), 7.21-7.27 (m, 1H), 7.44 (s, 1H), 7.51 (s, 1H), 7.96-8.07 (m, 1H), 8.29-8.38 (m, 1H), 13.42 (s, 1H), 7.66-7.71 (m, 2H), 7.86-7.91 (m, 2H) | 415 (M + H)⁺ |
| 22 | 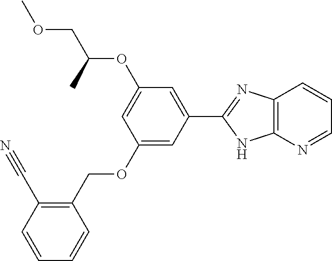 | ¹H NMR (400 MHz, DMSO) δ 1.29 (d, 3H), 3.25-3.35 (s + water, 3H), 3.46-3.60 (m, 2H), 4.70-4.80 (m, 1H), 5.36 (s, 2H), 6.80 (s, 1H), 7.26 (dd, 1H), 7.49 (s, 1H), 7.55 (s, 1H), 7.57-7.64 (m, 1H), 7.76-7.82 (m, 2H), 7.91- 7.97 (m, 1H), 7.97-8.13 (m, 1H), 8.29-8.45 (m, 1H), 12.99-13.58 (m, 1H) | 415 (M + H)⁺ |
| 23 | 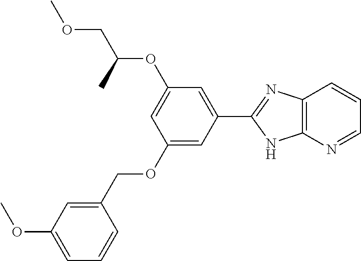 | ¹H NMR (400 MHz, DMSO) δ 1.27 (d, 3H), 3.27-3.33 (m, 3H), 3.46-3.58 (m, 2H), 3.78 (s, 3H), 4.69-4.78 (m, 1H), 5.19 (s, 2H), 6.75 (s, 1H), 6.89-6.94 (m, 1H), 7.04-7.10 (m, 211), 7.22-7.28 (m, 1H), 7.29-7.36 (m, 1H), 7.44 (s, 1H), 7.51 (s, 1H) | 420 (M + H)⁺ |
| 24 | 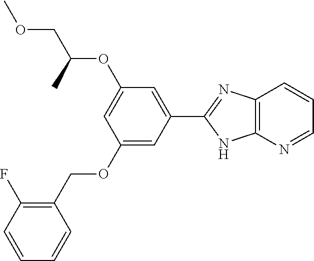 | ¹H NMR (400 MHz, DMSO) δ 1.26 (d, 3H), 3.31 (s, 3H), 3.45-3.56 (m, 2H), 4.68-4.77 (m, 1H), 5.23 (s, 2H), 6.76 (s, 1H), 7.21- 7.31 (m, 3H), 7.39-7.47 (m, 3H), 7.50 (s, 1H), 7.57-7.63 (m, 1H), 7.92-8.10 (m, 1H), 8.29- 8.40 (m, 1H), 13.42 (s) | 408 (M + H)⁺ |
| 25 | 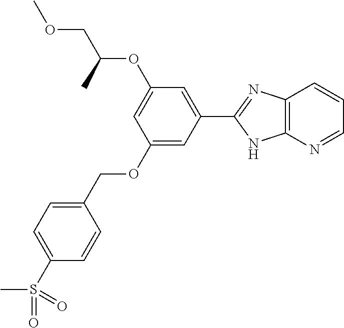 | ¹H NMR (400 MHz, DMSO) δ 1.27 (d, 3H), 3.23 (s, 3H), 3.27-3.37 (s + water, 3H), 3.46- 3.57 (m, 2H), 4.69-4.80 (m, 1H), 5.36 (s, 2H), 6.77 (s, 1H), 7.21-7.31 (m, 1H), 7.47 (s, 1H), 7.53 (s, 1H), 7.77 (d, 2H), 7.98 (d, 2H), 8.01-8.11 (m, 1H), 8.29-8.45 (m, 1H), 13.07- 13.57 (m, 1H) | 468 (M + H)⁺ |
| 26 | 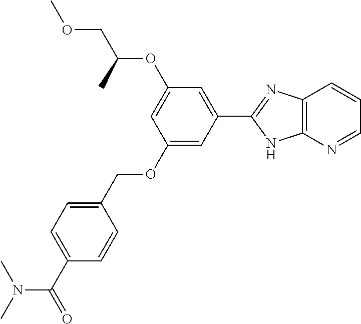 | ¹H NMR (400 MHz, DMSO) δ 1.27 (d, 3H), 2.88-3.05 (m, 4H), 3.27-3.40 (s + water, 3H), 3.47-3.58 (m, 2H), 4.69-4.78 (m, 1H), 5.23- 5.28 (m, 2H), 6.73-6.81 (m, 1H), 7.22-7.29 (m, 1H), 7.41-7.59 (m, 6H), 7.91-8.09 (m, 1H), 8.30-8.43 (m, 1H), 13.07-13.51 (m, 1H) | 461 (M + H)⁺ |

### EXAMPLE 27

### (2S)-2-{3-(3H-Imidazol-4,5-b]pyridin-2-yl)-5-[4-(methylsulfonyl)phenoxy]phenoxy}propan-1-ol

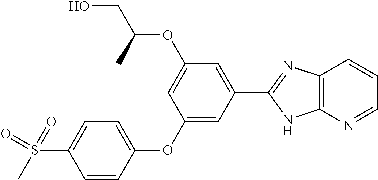

A solution of 2-{3-[(1S)-2-methoxy-1-methylethoxy]-5-[4-(methylsulfonyl)phenoxy]phenyl}-3H-imidazo[4,5-b]pyridine (Example 3; 100 mg, 0.22 mmol) in dry acetonitrile (2 mL) was treated with trimethylsilyl iodide (2.2 ml, 11 mmol) and stirred for 24 h under argon at room temperature. The reaction mixture was poured onto methanol to quench, and the resulting mixture then concentrated in vacuo. The residue was purified by flash column chromatography (CombiFlash Companion™ eluting with DCM containing 0-10% methanol). The product was re-chromatographed by preparative HPLC to give the title product as a clear oil, (62 mg, 64% yield). ¹H NMR (400 MHz, DMSO) δ 8.44 (d, 1H), 8.17 (d, 1H), 7.98 (d, 2H), 7.75 (s, 1H), 7.56 (s, 1H), 7.37 (d, 1H), 7.32 (dt, 2H), 7.00 (s, 1H), 4.62 (m, 1H), 3.57 (dd, 2H), 3.27 (s, 3H), 1.28 (d, 3H), LCMS: ink: 440 (M+H)⁺.

### EXAMPLE 28

### (2S)-2-{3-(6-Bromo-3H-imidazo[4,5-b]pyridin-2-yl)-5-[4-(methylsulfonyl)phenoxy]phenoxy}propan-1-ol

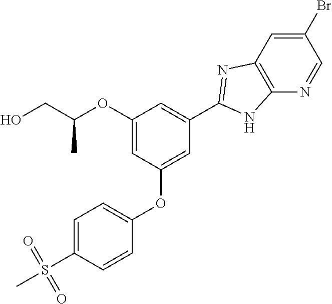

The title compound was prepared by a procedure essentially similar to that described for Example 27 starting from 6-bromo-2-{3-[(1S)-2-methoxy-1-methylethoxy]-5-[4-(methylsulfonyl)phenoxy]phenyl}-3H-imidazo[4,5-b]pyridine (Example 6), ¹H NMR (400 MHz, DMSO) δ 1.28 (d, 3H), 3.23 (s, 3H), 3.50-3.64 (m, 2H), 4.55-4.65 (m, 1H), 6.92-6.96 (m, 1H), 7.31 (dd, 2H), 7.52 (s, 1H), 7.74 (s, 1H), 7.97 (dd, 2H), 8.29 (s, 1H), 8.41-8.46 (m, 1H), m/z: 516, 518 (M−H), Br isotope pattern observed.

The following examples were made according to the method of Example 3, using a Biotage™ Initiator Sixty Microwave heater, heating from 15 mins to 2 hrs, in solvent such as acetonitrile, butyronitrile, DMF, DMA or NMP, and at a temperature from 100° C.-200° C.

| Ex No | Structure | NMR | M/z |
|---|---|---|---|
| 29 | 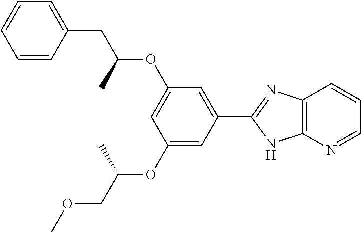 | ¹H NMR (400 MHz, DMSO) δ 1.24-1.34 (6H, m), 2.87-2.98 (1H, m), 3.01-3.09 (1H, m), 3.36 (3H, s), 3.46-3.52 (1H, m), 3.52-3.60 (1H, m), 4.62-4.69 (1H, m), 4.74-4.83 (1H, m), 6.57-6.64 (1H, m), 7.17-7.25 (2H, m), 7.26-7.35 (4H, m), 7.35-7.45 (2H, m), 7.94 (1H, d), 8.35 (1H, d), 12.95 (1H, d) | 418 (M + H)⁺ |
| 30 | 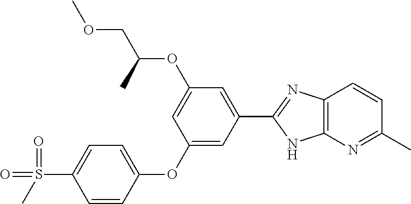 | ¹H NMR (300 MHz, CDCl₃) δ 1.46 (d, 3H), 2.98 (s, 3H), 3.21 (s, 3H), 3.57 (s, 3H), 3.72 (m, 2H), 4.82 (dq, 1H), 6.92 (s, 1H), 7.24 (d, 2H), 7.32 (d, 1H), 7.52 (s, 1H), 7.62 (s, 1H), 8.00 (d, 2H), 8.44 (d, 1H), 10.60 (s, 1H) | 468 (M + H)⁺ |
| 31 | 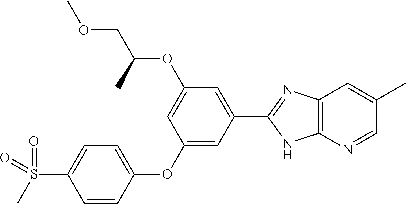 | ¹H NMR (300 MHz, CDCl₃) δ 1.47 (d, 1H), 2.67 (s, 3H), 3.21 (s, 3H), 3.57 (s, 3H), 3.72 (m, 2H), 4.88 (s, 1H), 6.92 (s, 1H), 7.25 (d, 2H), 7.52 (s, 1H), 7.66 (s, 1H), 8.00 (d, 2H), 8.20 (s, 1H), 8.35 (s, 1H) | 468 (M + H)⁺ |
| 32 | 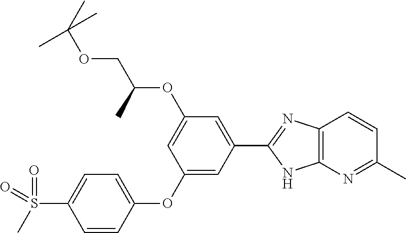 | ¹H NMR (400 MHz, CDCl₃) δ 1.17 (s, 9H), 1.27 (d, 3H), 2.60 (s, 3H), 3.07 (s, 3H), 3.38 (dd, 1H), 3.56 (dd, 1H), 4.46 (quintet, 1H), 6.75 (s, 1H), 7.04 (d, 1H), 7.09 (d, 2H), 7.44 (s, 1H), 7.53 (s, 1H), 7.83 (d, 2H), 7.95 (s, 1H) | 510 (M + H)⁺ |
| 33 | 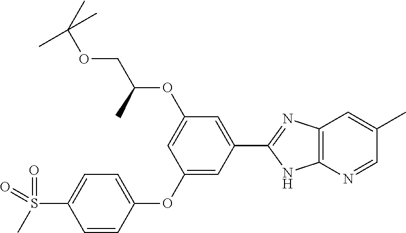 | ¹H NMR (400 MHz, CDCl₃) δ 1.19 (s, 9H), 1.40 (d, 3H), 2.48 (s, 3H), 3.11 (s, 3H), 3.50 (td, 1H), 3.61 (td, 1H), 4.64 (s, 1H), 6.86 (t, 1H), 7.19 (d, 2H), 7.63 (s, 1H), 7.76 (s, 1H), 7.91 (q, 3H), 8.34 (s, 1H) | 510 (M + H)⁺ |
| 34 | 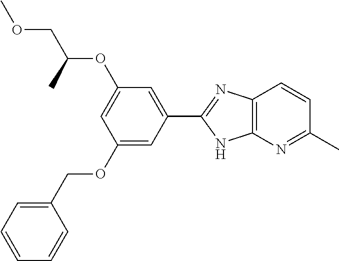 | ¹H NMR (400 MHz, CDCl₃) δ 1.38 (d, 3H), 2.78 (s, 3H), 3.50 (s, 3H), 3.63 (d, 2H), 4.71 (dq, 1H), 5.20 (s, 2H), 6.78 (s, 1H), 7.00 (d, 1H), 7.33 (d, 1H), 7.37 (d, 2H), 7.43 (t, 2H), 7.51 (d, 2H), 8.09 (d, 1H) | 402 (M + H)⁺ |
| 35 (1) | 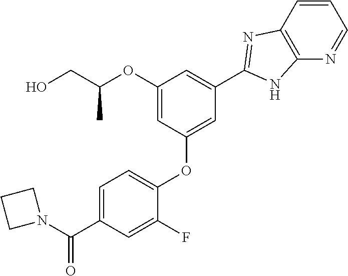 | ¹H NMR (400 MHz, CD₃OD) δ 1.28 (d, 3H), 2.33 (m, 2H), 3.65 (d, 2H), 4.17 (t, 2H), 4.41 (t, 2H), 4.58 (m, 1H), 6.89 (s, 1H), 7.23 (t, 1H), 7.41 (s, 1H), 7.47 (d, 1H), 7.55 (d, 1H), 7.60 (m, 2H), 8.38 (d, 1H), 8.50 (d, 1H) | 463 (M + H)⁺ |
| 36 (1) | 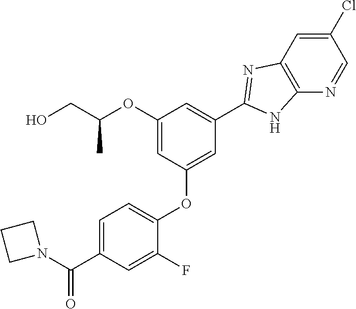 | ¹H NMR (400 MHz, CD₃OD) δ 1.35 (d, 3H), 2.41 (m, 2H), 3.71 (d, 2H), 4.23 (dd, 2H), 4.46 (dd, 2h), 4.63 (m, 1H), 6.91 (s, 1H), 7.27 (t, 1H), 7.42 (s, 1H), 7.52 (d, 1H), 7.61 (m, 2H), 8.11 (s, 1H), 8.47 (s, 1H) | 497 (M + H)⁺ |
| 37 | 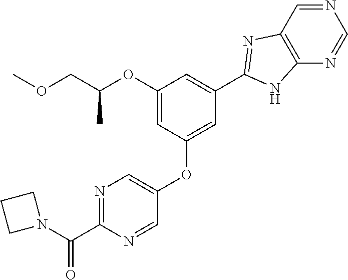 | ¹H NMR (400 MHz, DMSO-d₆) δ 1.30 (d, 3H), 2.29 (quintet, 2H), 3.32 (s, 3H), 3.48- 3.60 (m, 2H), 4.10 (t, 2H), 4.44 (t, 2H), 4.79 (sextet, 1H), 7.08 (s, 1H), 7.57 (s, 1H), 7.74 (s, 1H), 8.81 (s, 2H), 8.91 (s, 1H), 9.12 (s, 1H), 13.90 (s, 1H) | 462 (M + H)⁺ |
| 38 | 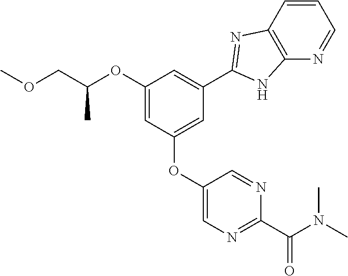 | ¹H NMR (400 MHz, DMSO-d₆) δ 1.30 (d, 3H), 2.86 (s, 3H), 3.04 (s, 3H), 3.33 (s, 3H), 3.47-3.59 (m, 2H), 4.80 (sextet, 1H), 7.06 (t, 1H), 7.34-7.37 (m, 1H), 7.46- 7.58 (m, 1H), 7.68-7.74 (m, 1H), 8.10- 8.23 (m, 1H), 8.39-8.49 (m, 1H), 8.75- 8.83 (m, 2H), 9.88 (s, 1H) | 449 (M + H)⁺ |
| 39 | 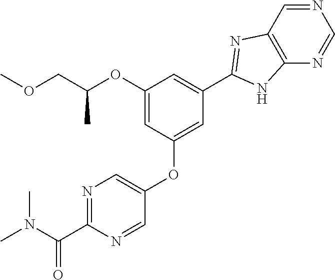 | ¹H NMR (400 MHz, DMSO-d₆) δ 1.30 (d, 3H), 2.86 (s, 3H), 3.04 (s, 3H), 3.32 (s, 3H), 3.50-3.59 (m, 2H), 4.80 (sextet, 1H), 7.08 (t, 1H), 7.58 (s, 1H), 7.73 (s, 1H), 8.80 (s, 2H), 8.91 (s, 1H), 9.12 (s, 1H), 13.91 (s, 1H) | 450 (M + H)⁺ |
| 40 | 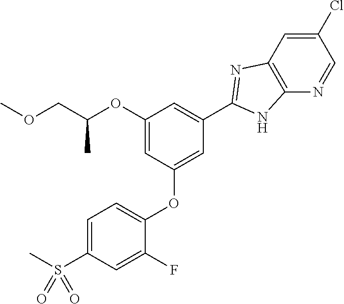 | ¹H NMR (400 MHz, DMSO-d₆) δ 1.29 (d, 3H), 3.30 (s, 3H), 3.32 (s, 3H), 3.48-3.59 (m, 2H), 4.73-4.84 (m, 1H), 6.96 (s, 1H), 7.39-7.53 (m, 2H), 7.70 (s, 1H), 7.81 (d, 1H), 8.03 (d, 1H), 8.18 (s, 1H), 8.37 (s, 1H), 13.38-14.08 (br s, 1H) | 547 (MH + MeCN)⁺ |
| 41 | 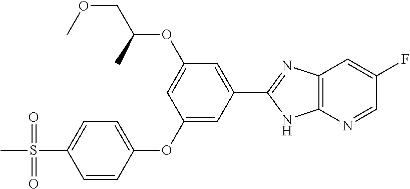 | ¹H NMR (400 MHz, DMSO-d₆) δ 1.30 (3H, d), 3.23 (3H, s), 3.32 (3H, s), 3.50-3.58 (2H, m), 4.76-4.80 (1H, m), 6.95 (1H, t), 7.29-7.33 (2H, m), 7.52 (1H, t), 7.70 (1H, d), 7.96-7.99 (3H, m), 8.37 (1H, t), 13.50 (1H, bs) | 472 (M + H)⁺ |
| 42 | 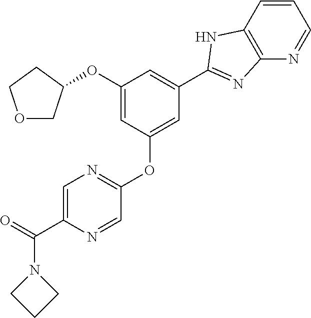 | ¹H NMR (400 MHz, CDCl₃) δ 2.14-2.18 (2H, m), 2.31-2.39 (2H, m), 3.86-4.06 (4H, m), 4.23 (2H, t), 4.64 (2H, t), 4.96 (1H, s), 6.81 (1H, t), 7.14-7.17 (1H, m), 7.68 (1H, s), 7.73 (1H, s), 8.01 (1H, d), 8.25- 8.29 (1H, m), 8.33-8.34 (1H, m), 8.74 (1H, s) | 459 (M + H)⁺ |
| 43 | 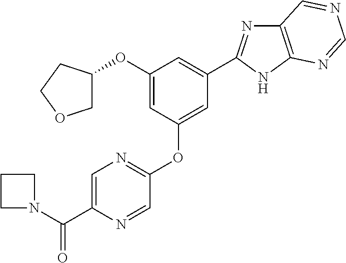 | ¹HNMR (400 MHz, DMSO-d₆) δ 2.10 (m, 1H), 2.36 (m, 3H), 3.85 (m, 1H), 3.93 (m, 2H), 3.99 (m, 1H), 4.16 (t, 2H), 4.63 (t, 2H), 5.23 (m, 1H), 7.19 (t, 1H), 7.76 (m, 1H), 7.79 (m, 1H), 8.68 (d, 1H), 8.76 (d, 1H), 8.98 (s, 1H), 9.18 (s, 1H), 13.71 (s, 1H) | 460 (M + H)⁺ |
| 44 | 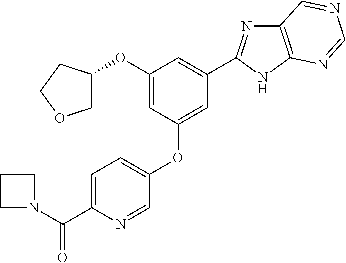 | ¹H NMR (400 MHz, DMSO-d₆) δ 2.10 (m, 1H), 2.35 (m, 3H), 3.85 (m, 1H), 3.93 (m, 2H), 4.00 (m, 1H), 4.14 (t, 2H), 4.66 (t, 2H), 5.25 (m, 1H), 7.05 (s, 1H), 7.59 (s, 1H), 7.71 (m, 1H), 7.74 (s, 1H), 8.09 (d, 1H), 8.55 (d, 1H), 8.97 (s, 1H), 9.18 (s, 1H), 13.14 (s, 1H) | 459 (M + H)⁺ |

(1) These Examples were prepared from the intermediate 3-[4-(Azetidin-1-ylcarbonyl)-2-fluorophenoxy]-5-((1S)-2- {[tert-butyl(dimethyl)silyly]oxy}-1-methylethoxy)benzoic acid; the silyl protecting group was removed thermally under the conditions of the reaction.

### EXAMPLE 29

2-{3-[(1S)-2-methoxy-1-methylethoxy]-5-[(1S)-1-methyl-2-phenylethoxy]phenyl}-3H-imidazo[4,5-b]pyridine

### EXAMPLE 30

2-{3-[(1S)-2-methoxy-1-methylethoxy]-5-[4-(methylsulfonyl)phenoxy]phenyl}-5-methyl-3H-imidazo[4,5-b]pyridine

### EXAMPLE 31

2-{3-[(1S)-2-methoxy-1-methylethoxy]-5-[4-(methylsulfonyl)phenoxy]phenyl}-6-methyl-3H-imidazo[4,5-b]pyridine

### EXAMPLE 32

2-{3-[(1S)-2-tert-butoxy-1-methylethoxy]-5-[4-(methylsulfonyl)phenoxy]phenyl}-5-methyl-3H-imidazo[4,5-b]pyridine

### EXAMPLE 33

2-{3-[(1S)-2-tert-butoxy-1-methylethoxy]-5-[4-(methylsulfonyl)phenoxy]phenyl}-6-methyl-3H-imidazo[4,5-b]pyridine

### EXAMPLE 34

2-{3-(benzyloxy)-5-[(1S)-2-methoxy-1-methylethoxy]phenyl}-5-methyl-3H-imidazo[4,5-b]pyridine

### EXAMPLE 35

(2S)-2-[3-[4-(azetidin-1-ylcarbonyl)-2-fluorophenoxy]-5-(3H-imidazo[4,5-b]pyridin-2-yl)phenoxy]propan-1-ol

### EXAMPLE 36

(2S)-2-[3-[4-(azetidin-1-ylcarbonyl)-2-fluorophenoxy]-5-(6-chloro-3H-imidazo[4,5-b]pyridin-2-yl)phenoxy]propan-1-ol

### EXAMPLE 37

8-{3-{[2-(azetidin-1-ylcarbonyl)pyrimidin-5-yl]oxy}-5-[(1S)-2-methoxy-1-methylethoxy]phenyl}-9H-purine

### EXAMPLE 38

5-{3-(3H-imidazo[4,5-b]pyridin-2-yl)-5-[(1S)-2-methoxy-1-methylethoxy]phenoxy}-N,N-dimethylpyrimidine-2-carboxamide

### EXAMPLE 39

5-[3-[(1S)-2-methoxy-1-methylethoxy]-5-(9H-purin-8-yl)phenoxy]-N,N-dimethylpyrimidine-2-carboxamide

### EXAMPLE 40

6-chloro-2-{3-[2-fluoro-4-(methylsulfonyl)phenoxy]-5-[(1S)-2-methoxy-1-methylethoxy]phenyl}-3H-imidazo[4,5-b]pyridine

### EXAMPLE 41

6-fluoro-2-{3-[(1S)-2-methoxy-1-methylethoxy]-5-[4-(methylsulfonyl)phenoxy]phenyl}-3H-imidazo[4,5-b]pyridine

### EXAMPLE 42

2-{3-{[5-(azetidin-1-ylcarbonyl)pyrazin-2-yl]oxy}-5-[(3S)-tetrahydrofuran-3-yloxy]phenyl}-1H-imidazo[4,5-b]pyridine

### EXAMPLE 43

8-{3-{[5-(azetidin-1-ylcarbonyl)pyrazin-2-yl]oxy}-5-[(3S)-tetrahydrofuran-3-yloxy]phenyl}-9H-purine

### EXAMPLE 44

8-{3-{[6-(azetidin-1-ylcarbonyl)pyridin-3-yl]oxy}-5-[(3S)-tetrahydrofuran-3-yloxy]phenyl}-9H-purine

Preparation of 2,3-diamino-5-methylpyridine is described in: Cai, Sui Xiong et al, Journal of Medicinal Chemistry (1997), 40(22), 3679-3686.

Preparation of 2,3-diamino-6-methylpyridine is described in: Keenan, Richard M. et al, Bioorganic & Medicinal Chemistry Letters (1998), 8(22), 3171-3176.

Preparation of 2,3-diamino-5-fluoropyridine is described in PCT Int. Appl. (2004), WO 2004092166 A2

The requisite methyl 3(S)-(1-methoxypropan-2-yloxy)-5(S)-(1-phenylpropan-2-yloxy)benzoate for Example 29 was prepared as follows:

### Methyl 3(S)-(1-methoxypropan-2-yloxy)-5(S)-(1-phenylpropan-2-yloxy)benzoate

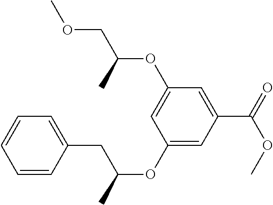

A solution of (S) methyl 3-hydroxy-5-(1-methoxypropan-2-yloxy)benzoate (7.2 g, 30 mmol), triphenyl phosphine (11.8 g, 45 mmol) and (R) 1-phenylpropan-2-ol (6.2 ml, 45 mmol) in THF was cooled in an ice-bath under an argon atmosphere and treated dropwise with a solution of DEAD in toluene (20 ml of a 40% solution, 45 mmol), keeping the temperature of the reaction below 10° C. After stirring for several hours, allowing to warm to ambient temperature, the solution was evaporated and the residue taken up in a hexane/ethyl acetate mixture (80 ml of 1:1). This was allowed to stand overnight and the precipitated material (mostly triphenyl phosphine oxide) removed by filtration. The residue was evaporated and chromatographed (200 g silica cartridge, gradient eluting with hexane containing 5-10% of ethyl acetate) to give the title compound as a colourless oil (9 g).

¹H NMR (400 MHz, DMSO) δ 1.20 (m, 6H), 2.8-3.0 (m, 2H), 3.25 (s, 3H) [NB. this signal was directly superimposed on signal due to HOD], 3.4-3.5 (m, 2H), 3.8 (s, 3H), 4.55-4.65 (m, 1H), 4.7-4.8 (m, 1H), 6.85 (s, 1H), 7.0 (s, 2H), 7.2 (m, 1H), 7.3 (m, 4H).

The requisite 3(S)-(1-methoxypropan-2-yloxy)-5(S)-(1-phenylpropan-2-yloxy)benzoic acid was prepared as follows:

### 3(S)-(1-methoxypropan-2-yloxy)-5(S)-(1-phenylpropan-2-yloxy)benzoic acid

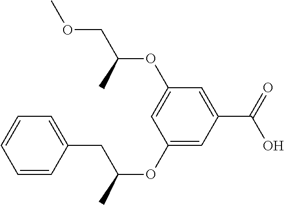

A solution of methyl 3(S)-(1-methoxypropan-2-yloxy)-5(S)-(1-phenylpropan-2-yloxy)benzoate (7.7 g, 21.5 mmol) in methanol/THF (100 ml of a 1:1 mixture) was treated with a solution of sodium hydroxide (2.15 g, 53.75 mmol) in water, and the reaction mixture stirred overnight at ambient temperature. The resulting solution was treated with a slight excess of 1M hydrochloric acid, and most of the organic solvent removed in vacuo; the aqueous residue was extracted twice with ethyl acetate. The combined extracts were washed twice with water, once with brine, dried (MgSO₄) and evaporated to give the title compound, 7.3 g, ¹H NMR (400 MHz, DMSO) δ 1.20 (m, 6H), 2.8-3.0 (m, 2H), 3.4 (m, 2H), 3.8 (s, 3H), 4.55-4.7 (m, 1H), 4.7-4.8 (m, 1H), 6.85 (s, 1H), 7.0 (s, 2H), 7.2 (m, 1H), 7.3 (m, 4H), m/z 343 (M−H), 345 (M+H)⁺.

The requisite methyl 3-hydroxy-5-[(2S)1-methoxypropan-2-yloxy]benzoate starting material can be prepared as described in: WO 2005121110 and WO 2005080359. The intermediate acid used for Examples 30 and 31 was described for Example 4.

The intermediate acid (3-[(1S)-2-tert-butoxy-1-methylethoxy]-5-[4-(methylsulfonyl)phenoxy]benzoic acid) used for Examples 32 and 33 was prepared as follows:

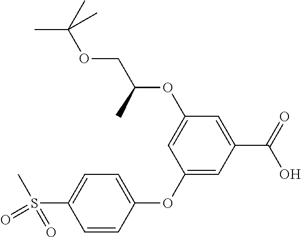

### 3-Fluoro-5-(4-methanesulfonyl-phenoxy)-benzonitrile

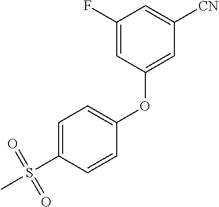

3,5-Difluorobenzonitrile (23.23 mmol; 3.23 g) was added to a 100 ml round bottomed flask followed by anhydrous potassium carbonate (17.42 mmol, 2.43 g), to this was added extra dry NMP (15.5 ml) and extra dry DMF (2 ml). The temperature was raised to 130° C. and the solution stirred the solution was light yellow in colour and after heating the reaction mixture became dark brown in colour. 4-Methanesulfonylphenol (11.61 mmol; 2.00 g) dissolved in NMP (2.5 ml) was added to the 3,5-difluorobenzonitrile by syringe pump over 1 hr and the mixture was stirred at 130° C. for 3 hrs. The reaction mixture was cooled to 60° C. and toluene (20 ml) was added, followed by water (20 ml). The two layers were separated and the aqueous/NMP/DMF layer was re-extracted with toluene (20 ml). The combined toluene extracts were washed with water (3×20 ml) to ensure all DMF and NMP were removed. The organic layer was then cooled from 60° C. to 20° C. over 4 hrs and an impurity was isolated by filtration, the toluene filtrate was distilled down to low volume (˜10 ml) and the residual white slurry was warmed 50° C., iso-hexane (40 ml) was charged and the temperature was reduced to 20° C. over 4 hrs. The product was isolated by filtration in good yield (2.81 g, 82.7%), ¹H NMR (400 MHz, CDCl₃) d 3.09 (s, 3H), 7.04 (d, 1H), 7.13 (s, 1H), 7.20 (m, 3H), 8.00 (d, 2H).

### 3-[(1S)-2-tert-Butoxy-1-methylethoxy]-5-[4-(methylsulfonyl)phenoxy]benzonitrile

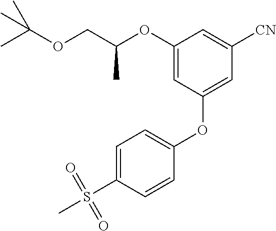

To a 3-necked round-bottomed flask (100 ml with condenser septum thermometer and magnetic follower) was charged sodium hydride (32.96 mmol; 1.32 g). The flask was inserted (vacuum/N₂ purges) and dry NMP (830.4 mmol; 80 ml) was charged. To the resulting suspension was charged (S)-1-tert-butoxy-2-propanol (30.21 mmol; 4.57 ml; 3.99 g) in 0.2 ml aliquots to control H₂ evolution. There was considerable frothing and a 2° C. rise in temperature was observed, the reaction mixture was cooled to 14° C. and then slowly warmed back to room temperature. Once gas evolution had ceased 3-fluoro-5-(4-methanesulfonyl-phenoxy)-benzonitrile (27.46 mmol; 8.0 g) was added in one portion. The reaction mixture was heated to 70° C. for 3 hours. The reaction was cooled to room temperature and toluene (240 ml) was charged followed by water (240 ml). The contents were stirred at room temperature for 30 minutes and then transferred to a separating funnel. The two layers were separated and the aqueous layer was further extracted with toluene (240 ml). The organic extracts were combined and washed once with sodium hydroxide (160 mmol; 160 ml) and then four times with water (4×160 ml). The toluene was removed in vacuo to leave an oil that slowly solidified (9.20 g; 83.02% yield), ¹H NMR (400 MHz, DMSO-d₆) 7.92 (d, 2H) 7.33 (s, 1H) 7.23 (d, 2H) 7.19 (s, 1H) 7.07 (t, 1H) 4.62 (m, 1H) 3.44 (m, 2H) 3.19 (s, 3H) (f) 1.21 (d, 3H) 1.07 (s, 9H)

### 3-[(1S)-2-tert-Butoxy-1-methylethoxy]-5-[4-(methylsulfonyl)phenoxy]benzoic acid

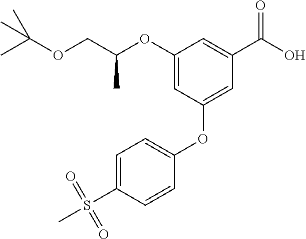

3-[(1S)-2-tert-butoxy-1-methylethoxy]-5-[4-(methylsulfonyl)phenoxy]benzonitrile (23.69 mmol; 9.56 g) was dissolved in ethanol (1.64 mol; 95.60 ml), and transferred to a 250 ml round bottomed flask, the residual solid was washed into the round bottomed flask with ethanol (85.88 mmol; 5.00 ml). Water (6.25 ml) and sodium hydroxide (118.46 mmol; 6.30 mL) was added and the reaction was heated to reflux for 18 hours. The ethanol was removed in vacuo. The residual yellow suspension was re-dissolved in MTBE (162.52 mL) and water (162.52 mL). The two layers were separated, the MTBE layer was discarded and the aqueous layer was acidified with 2M HCl (100 ml). The aqueous layer was extracted twice with MTBE (162.52 ml). The organic extracts were combined and dried with magnesium sulfate, the MTBE was removed in vacuo, to produce 7.0 g of the title product (69.93% yield), ¹H NMR (400 MHz, DMSO-d₆) 7.92 (d, 2H) 7.32 (s, 1H) 7.21 (d, 2H) 7.10 (s, 1H) 7.00 (t, 1H) 4.53 (m, 1H) 3.42 (m, 2H) 3.19 (s, 3H) 1.22 (d, 3H) 1.09 (s, 9H)

The required acid for Example 34 was described in Example 1.

The required acid starting material for Examples 35 and 36 {3-[4-(azetidin-1-ylcarbonyl)-2-fluorophenoxy]-5-((1S)-2-{[tert-butyl(dimethyl)silyl]oxy}-1-methylethoxy)benzoic acid} was prepared as described in WO2005/121110 Example 8.

The requisite acid starting material for Example 37, (S) 3-(2-azetidin-1-ylcarbonylpyrimidin-5-yl)oxy-5-(1-methoxypropan-2-yloxy)benzoic acid was prepared as follows:

### 5-Bromopyrimidine-2-carboxylic acid

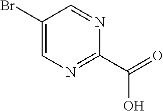

A mixture of 5-bromopyrimidine-2-carbonitrile (5.74 g, 31.20 mmol) and sodium hydroxide (3.75 g, 93.59 mmol) in water (100 ml) was heated at 60° C. for 1 hr. The reaction mixture was then acidified with 1M HCl and the volume of aqueous reduced in vacuo. The residue was extracted into 90:10 DCM:MeOH and the extracts evaporated to give a yellow solid. This was triturated with ethyl acetate was and the resulting yellow solid filtered off and washed well with more ethyl acetate. The filtrate was dried (MgSO₄), filtered, combined with the solid and the solvent removed in vacuo to give 5-bromopyrimidine-2-carboxylic acid as a yellow solid. 4.72 g, ¹H NMR (400 MHz, DMSO-d₆) δ 9.18 (s, 2H), 13.76 (s, 1H); the spectrum also shows signals due to an unidetified impurity, approx. 10 mol %.

### 2-(Azetidin-1-ylcarbonyl)-5-bromopyrimidine

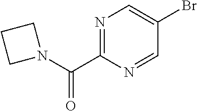

Oxalyl chloride (0.9 ml, 10 mmol) was added dropwise to a stirred suspension of 5-bromopyrimidine-2-carboxylic acid (1.7 g, 8.4 mmol) in DCM (15 ml). DMF (2 drops) was added and some frothing was observed. The yellow suspension was stirred at ambient temperature for 1 hr and then concentrated to a black oil which was re-dissolved in DCM (20 ml). A solution of azetidine hydrochloride (862 mg, 9.2 mmol) and triethylamine (2.6 ml, 18.4 mmol) in DCM (10 ml) was added slowly, and the resulting black reaction mixture was stirred overnight at ambient temperature. The reaction mixture was concentrated to a black oil (˜5 g); this was re-dissolved in DCM, filtered and purified by chromatography (120 g silica cartridge, using gradient elution with ethyl acetate containing 0-20% methanol to give the title product (1.02 g, 50% yield), ¹H NMR (400 MHz, DMSO-d₆) δ 2.33 (quintet, 2H), 4.14 (t, 2H), 4.44 (t, 2H), 9.17 (s, 2H), m/z 241 and 243 (M+H)⁺.

### (S) 3-{[2-(Azetidin-1-ylcarbonyl)pyrimidin-5-yl]oxy}-5-[(1S)-2-methoxy-1-methylethoxy]benzoic acid

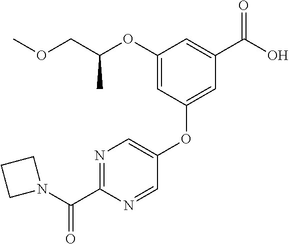

A mixture of 2-(azetidin-1-ylcarbonyl)-5-bromopyrimidine (870 mg, 3.6 mmol), methyl 3-hydroxy-5-[(2S)1-methoxypropan-2-yloxy]benzoate (prepared as described in WO 2005121110 and WO 2005080359—see Example 29) (863 mg, 3.6 mmol, 1 eq), caesium carbonate (2.9 g, 9.0 mmol) and bromotris(triphenylphosphine)copper (1) (1.0 g, 1.08 mmol) in DMA (20 ml) was heated for 2 hrs at 200° C. in the microwave oven. LCMS analysis of the crude reaction mixture showed the methyl ester of the product to have hydrolysed in situ. The reaction mixture was diluted with ethyl acetate and water, the layers separated, and the aqueous portion washed with three times ethyl acetate. The aqueous portion was then acidified with 2M HCl and extracted three times with ethyl acetate. The combined organic washings were washed with brine, dried (MgSO₄), and concentrated to give the title product as a yellow oil (1.02 g, 73%), ¹H NMR (400 MHz, DMSO-d₆) δ 1.23 (d, 3H), 2.27 (quintet, 2H), 3.30 (s, 3H), 3.43-3.52 (m, 2H), 4.09 (t, 2H), 4.42 (t, 2H), 4.71 (sextet, 1H), 7.12 (s, 1H), 7.21 (s, 1H), 7.33 (s, 1H), 8.72 (s, 2H), 13.03 (s, 1H), m/z 388 (M+H)⁺.

The requisite 3-[2-(dimethylcarbamoyl)pyrimidin-5-yl]oxy-5-(1-methoxypropan-2-yloxy)benzoic acid for Examples 38 and 39 was prepared as follows:

### 5-Bromo-N,N-dimethylpyrimidine-2-carboxamide

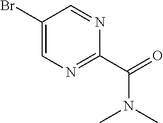

This was prepared using a method essentially similar to that given for 2-(azetidin-1-ylcarbonyl)-5-bromopyrimidine (above), using dimethylamine in place of azetidine, ¹H NMR (400 MHz, DMSO-d₆) δ 2.80 (s, 3H), 3.01 (s, 3H), 9.10 (s, 2H), m/z 230/232 (M+H)⁺.

### 3-({2-[(Dimethylamino)carbonyl]pyrimidin-5-yl}oxy)-5-[(1S)-2-methoxy-1-methylethoxy]benzoic acid

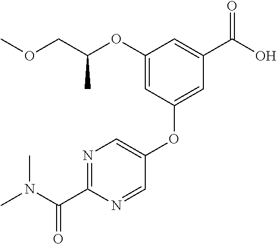

This was prepared using a method essentially similar to that given for 3-{[2-(azetidin-1-ylcarbonyl)pyrimidin-5-yl]oxy}-5-[(1S)-2-methoxy-1-methylethoxy]benzoic acid (above), using 5-bromo-N,N-dimethylpyrimidine-2-carboxamide in place of 2-(azetidin-1-ylcarbonyl)-5-bromopyrimidine, ¹H NMR (400 MHz, DMSO-d₆) δ 1.23 (d, 3H), 2.84 (s, 3H), 3.03 (s, 3H), 3.29 (s, 3H), 3.43-3.53 (m, 2H), 4.67-4.75 (m, 1H), 7.12 (t, 1H), 7.22 (dd, 1H), 7.33 (dd, 1H), 8.71 (s, 2H), 12.97 (s, 1H), m/z 376 (M+H)⁺.

The requisite 3-(2-fluoro-4-methylsulfonyl-phenoxy)-5-(1-methoxypropan-2-yloxy)benzoic acid for Example 40 was prepared as follows:

### 3-(2-Fluoro-4-methylsulfonyl-phenoxy)-5-[(2S)1-methoxypropan-2-yloxy]benzoic acid

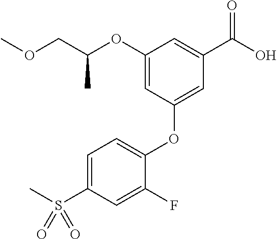

Methyl 3-(2-fluoro-4-methylsulfonyl-phenoxy)-5-[(2S) 1-methoxypropan-2-yloxy]benzoate was hydrolysed using a method essentially similar to that described in Example 4 to give 3-(2-fluoro-4-methylsulfonyl-phenoxy)-5-[(2S)1-methoxypropan-2-yloxy]benzoic acid, ¹H NMR (400 MHz, DMSO-d₆) δ 1.23 (d, 3H), 3.29 (s, 3H), 3.31 (s, 3H), 3.43-3.53 (m, 2H), 4.66-4.77 (m, 1H), 7.08 (d, 2H), 7.28-7.41 (m, 2H), 7.78 (d, 1H), 8.01 (d, 1H), 13.60 (s, 1H), m/z 397 (M−H)⁻.

The requisite methyl 3-(2-fluoro-4-methylsulfonyl-phenoxy)-5-[(2S)1-methoxypropan-2-yloxy]benzoate was prepared using a method essentially similar to that given in Example 4, starting from 1,2-difluoro-4-methylsulfonyl-benzene (commercially available) and methyl 3-hydroxy-5-[(2S)1-methoxypropan-2-yloxy]benzoate (see Example 4), ¹H NMR (400 MHz, DMSO-d₆) δ 1.2 (d, 3H), 3.3 (s, 3H), 3.3 (s, 3H), 3.4-3.5 (m, 2H), 3.85 (s, 3H), 4.7-4.8 (m, 1H), 7.05-7.15 (m, 2H), 7.3-7.4 (m, 2H), 7.75-7.85 (m, 1H), 8.0-8.1 (m, 1H) (the spectrum also contained signals due to ethyl acetate and unreacted difluoro starting material); m/z 435 (M+Na)⁺.

The requisite 3-{[5-(Azetidin-1-ylcarbonyl)pyrazin-2-yl]oxy}-5-[(3S)-tetrahydrofuran-3-yloxy]benzoic acid starting material for Examples 42 and 43 was prepared as follows:

### Methyl 3-[(phenylmethyl)oxy]-5-[(3S)-tetrahydrofuran-3-yloxy]benzoate

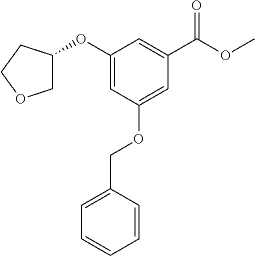

A mixture of methyl 3-hydroxy-5-{[phenylmethyl]oxy}benzoate (18.8 g, 72.75 mmol), (3R)-tetrahydrofuran-3-yl 4-methylbenzenesulfonate (prepared as described in Example 1) (18.5 g, 76.4 mmol) and potassium carbonate (20.08 g, 145.5 mmol) in butyronitrile (250 ml) was heated to 130° C. for 3 hours. The solvent was removed in vacuo and ethyl acetate added. The organics were washed with water (40 ml), 0.5M sodium hydroxide solution (40 ml), brine (40 ml), dried (MgSO₄), filtered and the solvent removed in vacuo. The residue was chromatographed on silica, eluting with a gradient of 0-5% methanol in DCM, to give to the desired compound as a colourless oil (20.1 g), ¹H NMR δ (CDCl₃): 2.08-2.26 (m, 2H), 3.78-4.01 (m, 4H), 3.90 (s, 3H), 4.92-4.96 (m, 1H), 5.08 (s, 2H), 6.69 (t, 1H), 7.15 (t, 1H), 7.29 (t, 1H), 7.34-7.44 (m, 5H); m/z 327 (M+H)⁺.

### Methyl 3-hydroxy-5-[(3S)-tetrahydrofuran-3-yloxy]benzoate

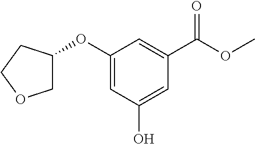

Methyl 3-[(phenylmethyl)oxy]-5-[(3S)-tetrahydrofuran-3-yloxy]benzoate (25.0 g, 76.2 mmol) was dissolved in THF (150 ml) and ethanol (150 ml). 10% Palladium on carbon (30 mg) was added and the mixture placed under a hydrogen atmosphere and left to stir at RT until the reaction was complete. The catalyst was removed by filtration through diatomaceous earth and the filtrate was concentrated in vacuo to give an orange oil which crystallised on standing. The solid was filtered off and washed with diethyl ether to give the desired product as a white solid (13.75 g), ¹H NMR δ (CDCl₃): 2.1-2.3 (2H, m), 3.9 (3H, s), 3.9-3.95 (2H, m), 3.97-4.05 (2H, m), 4.95 (1H, s), 5.6 (1), 6.6 (1H, t), 7.1 (1H, t), 7.13 (1H, t); m/z 237 (M+H)⁺

### Methyl 3-{[5-(azetidin-1-ylcarbonyl)pyrazin-2-yl]oxy}-5-[(3S)-tetrahydrofuran-3-yloxy]benzoate

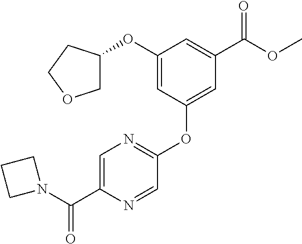

A solution of methyl 3-hydroxy-5-[(3S)-tetrahydrofuran-3-yloxy]benzoate (2.5 g, 10.5 mmol), potassium carbonate (2.9 g, 21.0 mmol) and 2-(azetidin-1-ylcarbonyl)-5-chloropyrazine (2.48 g, 12.6 mmol) in DMA (25 ml) was heated at 120° C. for 2 hours. The solution was diluted with ethyl acetate (150 ml), washed with water (3×50 ml), brine (20 ml), dried (MgSO₄), filtered and the solvent removed in vacuo. The residue was purified by chromatography on silica, eluting with 0-50% ethyl acetate in isohexane, to give the desired compound as a colourless oil (2.8 g), ¹H NMR δ (CDCl₃): 2.13-2.29 (m, 2H), 2.34-2.41 (m, 2H), 3.88-4.04 (m, 4H), 3.90 (s, 3H), 4.25 (t, 2H), 4.68 (t, 2H), 4.96-5.00 (m, 1H), 6.91 (t, 1H), 7.43 (d, 2H), 8.32 (d, 1H), 8.85 (d, 1H); m/z 386 (M+H)⁺, 384 (M−H)⁻

### 3-{[5-(Azetidin-1-ylcarbonyl)pyrazin-2-yl]oxy}-5-[(3S)-tetrahydrofuran-3-yloxy]benzoic acid

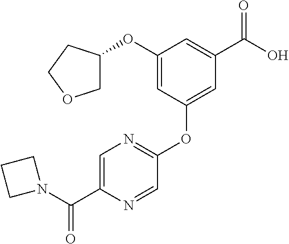

Methyl 3-{[5-(azetidin-1-ylcarbonyl)pyrazin-2-yl]oxy}-5-[(3S)-tetrahydrofuran-3-yloxy]benzoate (7.02 mmol) was dissolved in THF (50 ml), 1N sodium hydroxide (7.0 ml) was added followed by water (50 ml) and the resultant solution stirred at RT for 4 hours. The organics were removed in vacuo, the aqueous solution filtered and extracted with ethyl acetate (30 ml). The aqueous layer was acidified with 2N hydrochloric acid, extracted with ethyl acetate (3×50 ml), and the organic extracts washed with water (10 ml), brine (10 ml) then evaporated to dryness to give the desired material as a colourless foam (2.33 g), ¹H NMR δ (CDCl₃): 2.14-2.30 (m, 2H), 2.34-2.42 (m, 2H), 3.89-3.94 (m, 2H), 3.97-4.02 (m, 2H), 4.28 (t, 2H), 4.69 (t, 2H), 4.97-5.00 (m, 1H), 6.94 (t, 1H), 7.48 (t, 2H), 8.34 (d, 1H), 8.85 (d, 1H); m/z 386 (M+H)⁺, 384 (M−H)⁻

The requisite 2-(azetidin-1-ylcarbonyl)-5-chloropyrazine may be prepared as follows:

### 5-Chloropyrazine-2-carboxylic acid

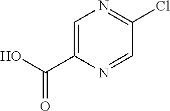

To a solution of methyl-5-chloropyrazine-2-carboxylate (120 mg, 0.70 mmol) in a mixture of acetonitrile (2 ml) and DMF (1 ml) was added lithium chloride (295 mg, 6.95 mmol). The suspension was heated to 160° C. for 5 mins in a Smith creator microwave after which time the reaction was diluted with water (10 ml). Saturated sodium bicarbonate solution (20 ml) was added and the aqueous layered extracted twice with ethyl acetate (30 ml). The combined organics were discarded and the aqueous layer adjusted to pH 4 with 1N hydrochloric acid. The aqueous phase was extracted twice with ethyl acetate (20 ml) and the combined organics washed with water (2×20 mlL), brine (10 ml) and dried (MgSO₄). The volatiles were removed to give the title compound as a colourless solid (68 mg), ¹H NMR δ (CDCl₃): 7.20 (1H, br s), 8.72 (1H, s), 9.21-9.21 (1H, m); m/z 157 (M−H)⁺.

### 2-(Azetidin-1-ylcarbonyl)-5-chloropyrazine

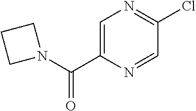

Oxalyl chloride (1.55 ml, 17.48 mmol), followed by DMF (2 drops), was added to a mixture of 5-chloropyrazine-2-carboxylic acid (2.31 g, 14.57 mmol) in DCM (40 ml). The reaction was stirred at RT for 2 hours after which time the volatiles were removed in vacuo. The residue was taken up DCM (40 ml) and azetidine (1.08 mL, 16.03 mmol) and triethylamine (4.46 ml, 32.06 mmol) added. The mixture was stirred at RT for 72 hours. The volatiles were removed in vacuo and ethyl acetate (100 ml) added to the residue. The organics were washed with water (100 ml), citric acid (50 ml), saturated sodium bicarbonate solution (50 ml), brine (50 ml), dried (MgSO₄), filtered and the solvent removed in vacuo to give a yellow solid. The residue was chromatographed on silica, eluting with a gradient of 50-100% ethyl acetate in iso-hexane, to give the desired compound as a yellow solid (2.38 g), ¹H NMR δ (CDCl₃): 2.35-2.42 (2H, m), 4.26 (2H, t), 4.67 (2H, t), 8.52 (1H, d), 9.09 (1H, d); m/z 198 (M+H)⁺.

The requisite 3-{[6-(Azetidin-1-ylcarbonyl)pyridin-3-yl]oxy}-5-[(3S)-tetrahydrofuran-3-yloxy]benzoic acid starting material for Example 44 was prepared as follows:

### 3-{[6-(Azetidin-1-ylcarbonyl)pyridin-3-yl]oxy}-5-[(3S)-tetrahydrofuran-3-yloxy]benzoic acid

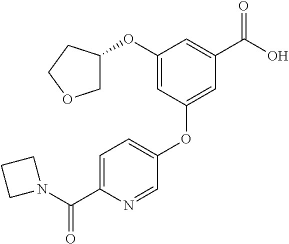

A mixture of methyl 3-hydroxy-5-[(3S)-tetrahydrofuran-3-yloxy]benzoate (500 mg, 2.10 mmol), 2-(azetidin-1-ylcarbonyl)-5-bromopyridine (605 mg, 2.52 mmol), copper (I) iodide (399 mg, 2.10 mmol) and 2,2,6,6 tetramethyl 3,5 heptanedione (1.8 mL, 8.40 mmol, 4.0 eq) and cesium carbonate (2.05 g, 6.30 mmol) in 1-methyl-2-pyrrolidinone (16 mL) was heated with stirring at 160° C. in a Microwave apparatus for 8 hours. LCMS indicated the presence of hydrolysed product with little starting material remaining. The reaction mixture was filtered through celite and washed through with DCM and methanol, and the filtrate and washings concentrated in vacuo. The residue was taken up into water and washed with ethyl acetate (3×30 mL); the aqueous phase was then acidified with 1N HCl and extracted with ethyl acetate (3×40 mL). The organic phase was washed with brine (20 mL), dried (MgSO₄) and concentrated in vacuo to give a brown residue. This was purified by chromatography on silica, eluting with 0-10% methanol in DCM, to give the title compound as a brown oil (330 mg, 41%). m/z 385 (M+H)⁺, RT 1.78 min.

The requisite methyl 3-hydroxy-5-[(3S)-tetrahydrofuran-3-yloxy]benzoate is an intermediate for Examples 42 and 43 and is described above.

The requisite 2-(azetidin-1-ylcarbonyl)-5-bromopyridine is described in WO 2005014571.

The following compounds were prepared using a method essentially similar to that described in Example 27, starting from the appropriate methyl ether (SM=starting material):

| Ex No | Structure | NMR | M/z |
|---|---|---|---|
| 45 SM = Example 3 | 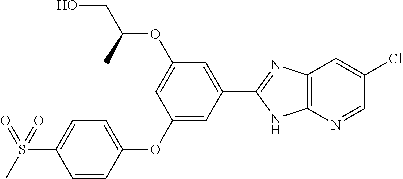 | ¹H NMR (400 MHz, DMSO-d₆) δ 1.28 (d, 3H), 3.23 (s, 3H), 3.64 (s, 2H), 4.60 (q, 1H), 6.94 (t, 1H), 7.31 (dd, 2H), 7.52 (t, 1H), 7.72 (t, 1H), 7.97 (dd, 2H), 8.18 (d, 1H), 8.38 (d, 1H) | 474 (M + H)⁺ |
| 46 SM = Example 30 | 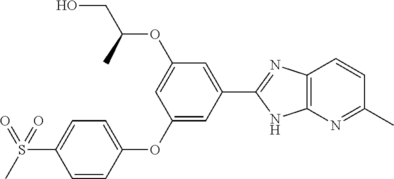 | ¹H NMR (400 MHz, CDCl₃) δ 1.57 (d, 1H), 3.08 (s, 3H), 3.30 (s, 3H), 4.06 (m, 2H), 4.93 (m, 1H), 6.99 (s, 1H), 7.37 (d, 2H), 7.50 (d, 1H), 7.69 (s, 1H), 7.83 (s, 1H), 8.13 (d, 2H), 8.53 (d, 9H) | 454 (M + H)⁺ |
| 48 SM = Example 86 | 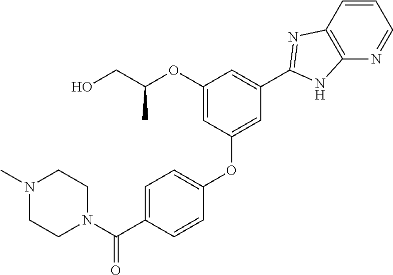 | ¹H NMR (400 MHz, DMSO-d₆) δ 1.28 (3H, d), 2.21 (3H, s), 2.33 (4H, s), 3.18 (3H, d), 3.25 (0H, s), 3.35-3.63 (2H, m), 4.06 (1H, q), 4.58 (1H, q), 4.89 (1H, t), 6.81 (1H, s), 7.14-7.17 (2H, m), 7.23- 7.27 (1H, m), 7.45-7.49 (3H, m), 7.67 (1H, s), 8.02 (1H, s), 8.35 (1H, s) | 488 (M + H)⁺ |
| 49 SM = Example 40 | 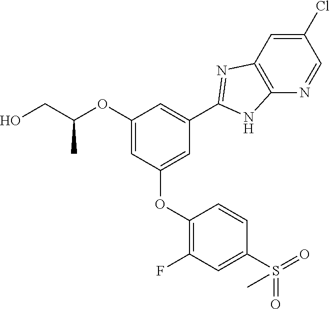 | ¹H NMR (400 MHz, DMSO-d₆) δ 1.19 (d, 3H), 3.21 (s, 3H), 3.42-3.55 (m, 2H), 4.52 (sextet, 1H), 4.79 (s, 1H), 6.85 (s, 1H), 7.35 (t, 1H), 7.39 (s, 1H), 7.62 (s, 1H), 7.72 (d, 1), 7.92-7.97 (m, 1H), 8.09 (s, 1H), 8.28 (d, 1H), 13.70 (s, 1H) | 492 (M + H)⁺ |
| 50 SM = Example 41 | 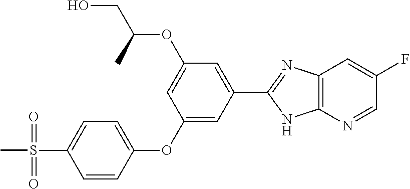 | ¹H NMR (400 MHz, DMSO-d₆) δ 1.29 (3H, d), 3.23 (3H, s), 3.52-3.64 (2H, m), 4.58-4.62 (1H, m), 4.89 (1H, t), 6.93 (1H, t), 7.29-7.33 (2H, m), 7.51 (1H, t), 7.71 (1H, t), 7.96-7.99 (3H, m), 8.37 (1H, t), 13.6 (1H, bs) | 456 (M − H)⁻ |
| 51 SM = Example 39 | 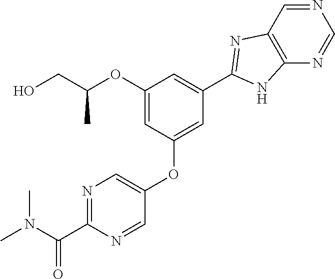 | ¹H NMR (400 MHz, DMSO-d₆) δ 1.29 (d, 3H), 2.86 (s, 3H), 3.04 (s, 3H), 3.51- 3.65 (m, 2H), 4.62 (sextet, 1H), 4.90 (t, 1H), 7.06 (s, 1H), 7.57 (s, 1H), 7.74 (s, 1H), 8.80 (s, 2H), 8.91 (s, 1H), 9.13 (s, 1H), 13.96 (s, 1H) | 436 (M + H)⁺ |
| 52 SM described below | 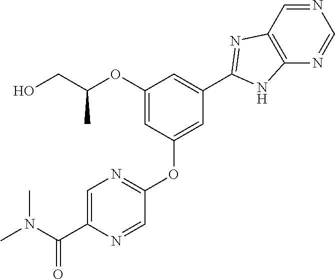 | ¹H NMR (400 MHz, DMSO-d₆) δ 1.29 (d, 3H), 3.05 (d, 6H), 3.50-3.66 (m, 2H), 4.60 (sextet, 1H), 4.91 (t, 1H), 7.10 (t, 1H), 7.69 (t, 1H), 7.78 (s, 1H), 8.46 (d, 1H), 8.60 (d, 1H), 8.90 (s, 1H), 9.10 (s, 1H), 13.76 (bs, 1H) | 436 (M + H)⁺ |

### EXAMPLE 45

(2S)-2-{3-(6-chloro-3H-imidazo[4,5-b]pyridin-2-yl)-5-[4-(methylsulfonyl)phenoxy]phenoxy}propan-1-ol

### EXAMPLE 46

(2S)-2-{3-(5-methyl-3H-imidazo[4,5-b]pyridin-2-yl)-5-[4-(methylsulfonyl)phenoxy]phenoxy}propan-1-ol

### EXAMPLE 47

there is no Example 47.

### EXAMPLE 48

(2S)-2-(3-(3H-imidazo[4,5-b]pyridin-2-yl)-5-{4-[(4-methylpiperazin-1-yl)carbonyl]phenoxy}phenoxy)propan-1-ol

### EXAMPLE 49

(2S)-2-{3-(6-chloro-3H-imidazo[4,5-b]pyridin-2-yl)-5-[2-fluoro-4-(methylsulfonyl)phenoxy]phenoxy}propan-1-ol

### EXAMPLE 50

(2S)-2-{3-(6-fluoro-3H-imidazo[4,5-b]pyridin-2-yl)-5-[4-(methylsulfonyl)phenoxy]phenoxy}propan-1-ol

### EXAMPLE 51

5-[3-[(1S)-2-hydroxy-1-methylethoxy]-5-(9H-purin-8-yl)phenoxy]-N,N-dimethylpyrimidine-2-carboxamide

### EXAMPLE 52

### 5-[3-[(1S)-2-hydroxy-1-methylethoxy]-5-(9H-purin-8-yl)phenoxy]-N,N-dimethylpyrazine-2-carboxamide

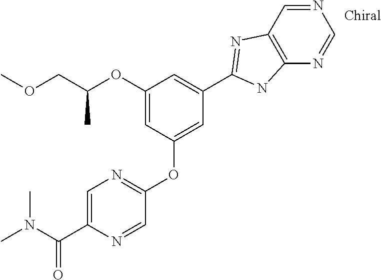

The requisite 5-[3-(1-methoxypropan-2-yloxy)-5-(9H-purin-8-yl)phenoxy]-N,N-dimethyl-pyrazine-2-carboxamide starting material for Example 52 was prepared by a method essentially similar to that described in Example 3, starting from 3-[5-(dimethylcarbamoyl)pyrazin-2-yl]oxy-5-[(2S)-1-methoxypropan-2-yl]oxy-benzoic acid and 4,5 diamino pyrimidine, ¹H NMR (400 MHz, CDCl₃) δ 1.36 (d, 3H), 3.18 (s, 3H), 3.21 (s, 3H), 3.44 (s, 3H), 3.53 (dd, 1H), 3.68 (dd, 1H), 4.75 (bs, 1H), 6.98 (s, 1H), 7.68 (s, 1H), 7.85 (s, 1H), 8.40 (s, 1H), 8.54 (s, 1H), 9.03 (bs, 1H), 9.23 (bs, 1H), 13.95 (s, 1H), m/z 450, (M+H)⁺.

The requisite 3-[5-(dimethylcarbamoyl)pyrazin-2-yl]oxy-5-[(2S)-1-methoxypropan-2-yl]oxy-benzoic acid was prepared as follows:

### 5-chloro-N,N-dimethyl-pyrazine-2-carboxamide

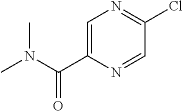

This was prepared in a manner essentially similar to that described for 2-(azetidin-1-ylcarbonyl)-5-chloropyrazine (see Examples 42 and 43), ¹H NMR (400 MHz, CD₃OD) δ 3.34 (s, 3H), 3.38 (s, 3H), 8.90 (s, 1H), 8.92 (s, 1H), m/z 186 (M+H)⁺.

The following compounds were made by an analogous method to Example 9.

| Ex No | Structure | NMR | M/z |
|---|---|---|---|
| 53 | 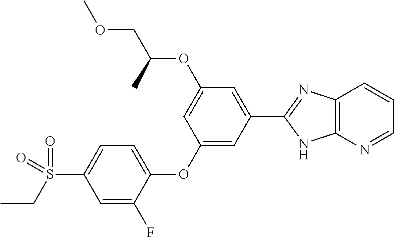 | ¹H NMR (400 MHz, CDCl₃) δ 1.32 (t, 3H), 1.36 (d, 3H), 3.16 (q, 2H), 3.47 (s, 2H), 3.61 (dd, 2H), 4.73 (dq, 1H), 6.74 (t, 1H), 7.22 (t, 1H), 7.43 (s, 1H), 7.49 (dd, 1H), 7.57 (s, 1H), 7.65 (d, 1H), 7.73 (dd, 1H), 8.39 (dd, 1H), 8.50 (dd, 1H) | 486 (M + H)⁺ |
| 54 | 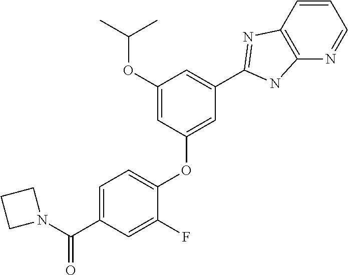 | ¹H NMR (400 MHz, DMSO-d₆) δ 1.34 (d, 6H), 2.28 (t, 2H), 4.06-4.13 (m, 2H), 4.33- 4.44 (m, 2H), 4.76 (quintet, 1H), 6.78 (s, 1H), 7.25 (dd, 1H), 7.30 (t, 1H), 7.40 (s, 1H), 7.53 (d, 1H), 7.59-7.71 (m, 2H), 7.93 (m, 1H), 8.29 (m, 1H) | 447 (M + H)⁺ |
| 55 | 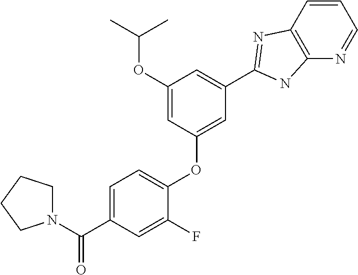 | ¹H NMR (400 MHz, DMSO-d₆) δ 1.34 (d, 6H), 1.77-1.94 (m, 4H), 3.42-3.54 (m, 4H), 4.77 (quintet, 1H), 6.82-6.86 (m, 1H), 7.32 (t, 1H), 7.36-7.47 (m, 3H), 7.59- 7.66 (m, 2H), 8.20 (d, 1H), 8.45 (d, 1H) | 461 (M + H)⁺ |
| 56 | 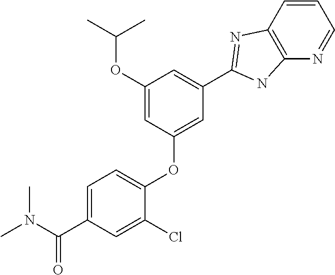 | ¹H NMR (400 MHz, DMSO-d₆) δ 1.34 (d, 6H), 2.99 (s, 611), 4.77 (quintet, 1H), 6.78- 6.82 (m, 1H), 7.27 (d, 1H), 7.34 (dd, 1H), 7.37-7.39 (m, 1H), 7.44-7.48 (m, 1H), 7.62-7.65 (m, 1H), 7.70 (d, 1H), 8.14 (d, 1H), 8.42 (d, 1H) | 451 (M + H)⁺ |

### Methyl 3-[5-(dimethylcarbamoyl)pyrazin-2-yl]oxy-5-[(2S)-1-methoxypropan-2-yl]oxy-benzoate

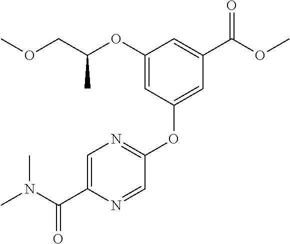

A solution of methyl 3-hydroxy-5-[(2S)1-methoxypropan-2-yloxy]benzoate (prepared as described in WO 2005121110 and WO 2005080359—see Example 29) (2.0 g, 8.32 mmol), caesium carbonate (5.4 g, 16.65 mmol) and 5-chloro-N,N-dimethyl-pyrazine-2-carboxamide (1.5 g, 8.32 mmol) in DMA (118 ml) was heated at 115° C. for 1 hour. The solvent was evaporated in vacuo and the residue purified by chromatography on silica (40 g cartridge), eluting with a gradient of isohexane/ethyl acetate (1:1 by volume) to neat ethyl acetate to give the title compound as a colourless oil (2.7 g), ¹H NMR (400 MHz, CDCl₃) δ 1.36 (d, 3H), 3.18 (s, 3H), 3.21 (s, 3H), 3.44 (s, 3H), 3.53 (dd, 1H), 3.61 (dd, 1H), 3.94 (s, 3H), 4.63 (m, 1H), 6.98 (dd, 1H), 7.43 (dd, 1H), 7.54 (dd, 1H), 8.37 (dd, 1H), 8.55 (dd, 1H), m/z 390 (M+H)⁺.

### 3-[5-(dimethylcarbamoyl)pyrazin-2-yl]oxy-5-[(2S)-1-methoxypropan-2-yl]oxy-benzoic acid

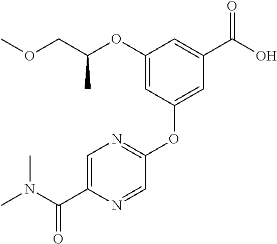

The title compound was prepared by base hydrolysis of methyl 3-[5-(dimethylcarbamoyl)pyrazin-2-yl]oxy-5-[(2S)-1-methoxypropan-2-yl]oxy-benzoate in a manner essentially similar to that described in Example 4, ¹H NMR (300 MHz, CDCl₃) δ 1.42 (d, 3H), 3.26 (s, 3H), 3.27 (s, 3H), 3.52 (s, 3H), 3.60 (dd, 1H), 3.69 (dd, 1H), 4.69 (m, 1H), 7.08 (dd, 1H), 7.54 (dd, 1H), 7.64 (dd, 1H), 8.45 (dd, 1H), 8.63 (dd, 1H), m/z 376 (M+H)⁺.

| | | | |
|---|---|---|---|
| 57 | 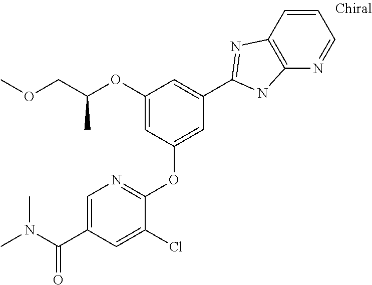 | ¹H NMR (400 MHz, DMSO-d₆) δ 1.30 (d, 3H), 2.99 (s, 6H), 3.33 (s, 3H), 3.49-3.59 (m, 2H), 4.77 (sextet, 1H), 7.02 (s, 1H), 7.23-7.29 (m, 1H), 7.60 (s, 1H), 7.73 (s, 1H), 7.95-8.08 (m, 1H), 8.17-8.23 (m, 2H), 8.31-8.39 (m, 1H), 13.46 (s, 1H) | 482 (M + H)⁺ |

### EXAMPLE 53

2-{3-[4-(ethylsulfonyl)-2-fluorophenoxy]-5-[(1S)-2-methoxy-1-methylethoxy]phenyl}-3H-imidazo[4,5-b]pyridine

### EXAMPLE 54

2-{3-[4-(azetidin-1-ylcarbonyl)-2-fluorophenoxy]-5-isopropoxyphenyl}-3H-imidazo[4,5-b]pyridine

### EXAMPLE 55

2-{3-[2-fluoro-4-(pyrrolidin-1-ylcarbonyl)phenoxy]-5-isopropoxyphenyl}-3H-imidazo[4,5-b]pyridine

### EXAMPLE 56

3-chloro-4-[3-(3H-imidazo[4,5-b]pyridin-2-yl)-5-isopropoxyphenoxy]-N,N-dimethylbenzamide

### EXAMPLE 57

5-chloro-6-{3-(3H-imidazo[4,5-b]pyridin-2-yl)-5-[(1S)-2-methoxy-1-methylethoxy]phenoxy}-N,N-dimethylnicotinamide

The preparations of:

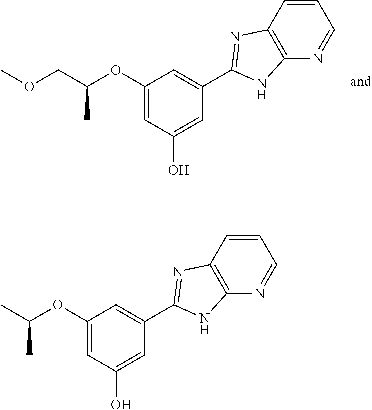

were described earlier.

The preparations of

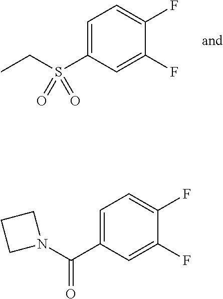

are both described in WO 2005121110 A1 and WO 2005080359 A1.

The preparations of

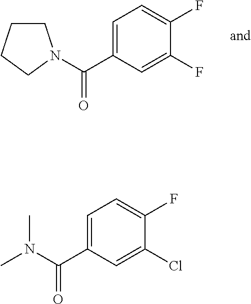

are both described in WO 2005121110 A1.

The requisite 5,6-dichloro-N,N-dimethyl-pyridine-3-carboxamide for Example 57 was prepared as follows:

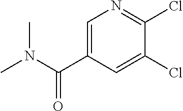

A suspension of 5,6 dichloropyridine-3-carboxylic acid (4.8 g, 25 mmol) in DCM (50 ml) was treated with oxalyl chloride (10.9 ml, 125 mmol) and the mixture stirred at ambient temperature with the addition of DMF (one drop). Complete solution was attained, which was then stirred for approximately 3 hrs. The solvent was removed in vacuo and the residue azeotroped once with more DCM, and then dried under high vacuum. More solvent was added (DCM, 60 ml), followed by dimethylamine (14 ml of a 2M solution in THF, 27.5 ml) and triethylamine 10.5 ml, 75 mmol). The reaction mixture was left to stand overnight and then washed twice with water and once with brine, dried (Phase Separating paper) and evaporated to give 5,6-dichloro-N,N-dimethyl-pyridine-3-carboxamide (4.3 g) as a pale brown solid which contained some triethylamine. The solid was dissolved in ethyl acetate and the solution was washed twice with water and once with brine, dried (MgSO₄) and evaporated to give 5,6-dichloro-N,N-dimethyl-pyridine-3-carboxamide (3.8 g) as a pale brown solid, ¹H NMR (400 MHz, DMSO) δ 2.94 (s, 3H), 3.00 (s, 3H), 8.24 (d, 1H), 8.46 (d, 1H), m/z 217 (M−H)⁻, 219 (M+H+)⁺.

### EXAMPLE 58

### 5-Methoxy-2-{3-[(1S)-2-methoxy-1-methylethoxy]-5-[4-(methylsulfonyl)phenoxy]phenyl}-1H-imidazo[4,5-b]pyrazine

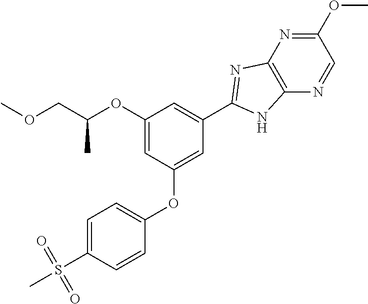

A mixture of 5-bromo-2-{3-[(1S)-2-methoxy-1-methylethoxy]-5-[4-(methylsulfonyl)phenoxy]phenyl}-1H-imidazo[4,5-b]pyrazine (150 mg, 0.28 mmol) and copper (I) iodide (110 mg, 0.56 mmol) in methanol (2 ml) was heated at 140° C. in a Biotage Initiator™ microwave for 5 mins. To the resulting solution sodium metal (46 mg, 2 mmol) was added portionwise and allowed to stir for 20 mins after addition. The mixture was then heated in the microwave for a further 10 mins. The resulting mixture was quenched with aqueous 2M HCl and the solvent removed in vacuo and the solid thus obtained was digested with ethyl acetate. The ethyl acetate extracts were filtered and the solvent removed from the filtrate. The red/brown gummy residue was purified by chromatography to give the title compound, 36 mg.

¹H NMR (400 MHz, DMSO-d₆) δ1.30 (3H, d), 3.23 (3H, s), 3.32 (3H, s), 3.50-3.58 (2H, m), 3.96 (3H, s), 4.74-4.81 (1H, m), 6.93 (1H, s), 7.29-7.33 (2H, m), 7.50 (1H, s), 7.68 (1H, s), 7.96-7.99 (2H, m), 8.0-8.1 (1H, m), 13.78 (1H, s), m/z 485 (M+H)⁺, 483 (M−H)⁻

The requisite 5-bromo-2-{3-[(1S)-2-methoxy-1-methylethoxy]-5-[4-(methylsulfonyl)phenoxy]phenyl}-1H-imidazo[4,5-b]pyrazine starting material was prepared as described in Example 7.

### EXAMPLE 59

### 2-{3-[(1S)-2-Methoxy-1-methylethoxy]-5-[4-(1,2,4-oxadiazol-3-yl)phenoxy]phenyl}-3H-imidazo[4,5-b]pyridine

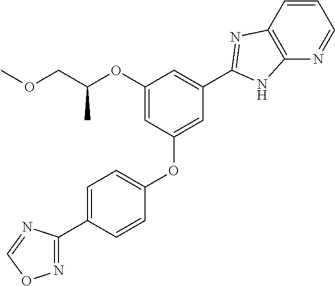

A solution of hydroxylamine (0.66 ml of 50% w/w) was added to a solution of 4-{3-(3H-imidazo[4,5-b]pyridin-2-yl)-5-[(1S)-2-methoxy-1-methylethoxy]phenoxy}benzonitrile (200 mg, 0.5 mmol) in ethanol (2.0 ml) and the resulting brown solution was stirred at ambient temperature over the weekend. The reaction mixture was then concentrated in vacuo and the residue redissolved in trimethyl orthoformate (2.0 ml). This solution was treated with boron trifluoride-diethyletherate (2 drops, catalytic) and heated at 60° C. for 80 mins in Biotage Initiator EXP60™ microwave. The reaction mixture was concentrated in vacuo and purified by reverse phase preparative HPLC to give the title compound, 21 mg (9%), ¹H NMR (400 MHz, DMSO-d₆) δ 1.23 (d, 3H), 3.25 (s, 3H), 3.42-3.52 (m, 2H), 4.67-4.74 (m, 1H), 6.84-6.88 (m, 1H), 7.21-7.29 (m, 3H), 7.43-7.46 (m, 1H), 7.61-7.63 (m, 1H), 8.01-8.07 (m, 3H), 8.32-8.36 (m, 1H), 9.63 (s, 1H), m/z 444 (M+H)⁺.

The requisite 4-{3-(3H-imidazo[4,5-b]pyridin-2-yl)-5-[(1S)-2-methoxy-1-methylethoxy]phenoxy}benzonitrile was prepared as described in Examples 16 or 62.

### EXAMPLE 60

### 2-{3-[(1S)-2-Methoxy-1-methylethoxy]-5-[4-(methylsulfonyl)phenoxy]phenyl}-3H-imidazo[4,5-b]pyridine-6-carbonitrile

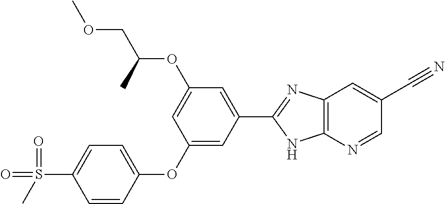

2-{3-[(1S)-2-methoxy-1-methylethoxy]-5-[4-(methylsulfonyl)phenoxy]phenyl}-3H-imidazo[4,5-b]pyridine-6-carboxamide (193 mg, 0.39 mmol) was dissolved in dioxane under an argon atmosphere. The solution was cooled to 0° C. and treated sequentially with trifluoroacetic anhydride (143 μl, 1.01 mmol) and pyridine (189 μl, 2.33 mmol). The reaction mixture allowed to come to room temperature overnight and was then concentrated in vacuo. The residue was purified by preparative HPLC to give the title compound, ¹H NMR (400 MHz, CDCl₃) δ 1.37 (d, 3H), 3.10 (s, 3H), 3.44 (s, 3H), 3.68 (m, 2H), 4.75 (m, 1H), 6.89 (s, 1H), 7.16 (d, 2H), 7.45 (s, 1H), 7.58 (s, 1H), 7.90 (d, 2H), 8.46 (s, 1H), 8.72 (s, 1H), m/z 477 (M−H)⁻.

The requisite 2-{3-[(1S)-2-methoxy-1-methylethoxy]-5-[4-(methylsulfonyl)phenoxy]phenyl}-3H-imidazo[4,5-b]pyridine-6-carboxamide starting material was made by a method essentially similar to that described for Example 3, starting with 3-[(1S)-2-methoxy-1-methylethoxy]-5-[4-(methylsulfonyl)phenoxy]benzoic acid [preparation described under Example 4] and 5,6-diaminonicotinamide, ¹H NMR (400 MHz, DMSO-d₆) δ 1.29 (d, 3H), 3.25 (s, 3H), 3.32 (s, 3H), 3.61 (m, 2H), 4.85 (m, 1H), 6.97 (s, 1H), 7.31 (d, 2H), 7.49 (s, 1H), 7.55 (d, 1H), 7.74 (s, 1H), 7.98 (d, 2H), 8.12 (s, 1H), 8.46 (d, 1H), 8.90 (d, 1H), m/z 497, (M+H)⁺.

### EXAMPLE 61

### N-[2-(Dimethylamino)ethyl]-6-{3-(3H-imidazo[4,5-b]pyridin-2-yl)-5-[(1S)-2-methoxy-1-methylethoxy]phenoxy}nicotinamide

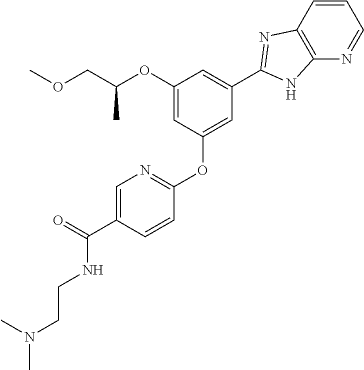

A solution of 5-chloro-N-[2-(dimethylamino)ethyl]-6-{3-(3H-imidazo[4,5-b]pyridin-2-yl)-5-[(1S)-2-methoxy-1-methylethoxy]phenoxy}nicotinamide (187 mg, 0.36 mmol) in an ethanol/methanol mixture (14 ml of 1:1) was treated with palladium-on-charcoal catalyst (94 mg of 10%), and the reaction mixture stirred overnight under an atmosphere of hydrogen (balloon).

The catalyst was filtered off, replaced with fresh, and the reaction mixture stirred at ambient temperature under hydrogen for a further 2 days. The reaction mixture was then filtered (glass fibre paper) and concentrated to a colourless oil (59 mg). This was purified by reverse phase HPLC the fractions treated with MP-Carbonate to neutralise the TFA, and then concentrated in vacuo to give the title compound, 25 mg (14%), ¹H NMR (400 MHz, DMSO-d₆) δ 1.30 (d, 3H), 2.54 (s, 6H), 3.33 (s, 3H), 3.43-3.61 (m, 6H), 4.77 (sextet, 1H), 6.94 (s, 1H), 7.21-7.28 (dd, 2H), 7.58 (s, 1H), 7.72 (s, 1H), 8.05 (d, 1H), 8.31 (d, 2H), 8.63 (s, 1H), 8.67 (s, 1H), 13.11-13.57 (m, 1H), m/z 491 (M+H)⁺.

The requisite 5-chloro-N-[2-(dimethylamino)ethyl]-6-{3-(3H-imidazo[4,5-b]pyridin-2-yl)-5-[(1S)-2-methoxy-1-methylethoxy]phenoxy}nicotinamide starting material was prepared in a manner essentially similar to that described in Example 9, starting from 3-(3H-imidazo[4,5-b]pyridin-2-yl)-5-[(1S)-2-methoxy-1-methylethoxy]phenol and 5,6-dichloro-N-[2-(dimethylamino)ethyl]nicotinamide, ¹H NMR (400 MHz, DMSO-d₆) δ 1.20 (d, 3H), 2.76 (d, 6H), 3.17 (q, 2H), 3.22 (s, 3H), 3.39-3.49 (m, 2H), 3.52 (q, 2H), 4.68 (sextet, 1H), 6.93 (t, 1H), 7.20 (dd, 1H), 7.49 (t, 1H), 7.64 (t, 1H), 7.96 (dd, 1H), 8.29 (dd, 1H), 8.37 (d, 1H), 8.46 (d, 1H), 8.75 (t, 1H), 9.24 (s, 1H), m/z 525 (M+H)⁺ for ³⁵Cl isotope.

### EXAMPLE 62

### 4-[{3-(3H-Imidazol-4,5-b]pyridin-2-yl)-5-[(1S)-2-methoxy-1-methylethoxy]phenoxy}benzoic acid

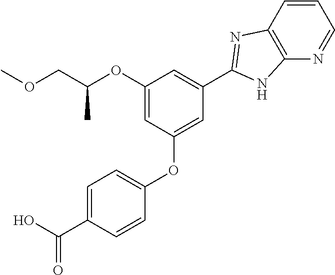

A solution of sodium hydroxide (150 mg, 3.75 mmol) in water (1 ml) and ethanol (5 ml) was added to 4-{3-(3H-imidazo[4,5-b]pyridin-2-yl)-5-[(1S)-2-methoxy-1-methylethoxy]phenoxy}benzonitrile (500 mg, 1.25 mmol), and the resulting brown solution was heated at 80° C. for 20 hours. The reaction mixture was cooled to ambient temperature and acidified with 2M HCl; ethyl acetate was added and the layers separated. The aqueous fraction was washed with three portions of ethyl acetate and the combined organics washed with brine, dried (MgSO₄) and concentrated in vacuo to give the title compound (461 mg, 88%), ¹H NMR (400 MHz, DMSO) δ 1.30 (d, 3H), 3.35 (s, 3H), 3.43-3.60 (m, 2H), 4.63-4.83 (m, 1H), 6.92 (s, 1H), 7.06-7.32 (m, 3H), 7.53 (s, 1H), 7.73 (s, 1H), 7.88-8.09 (m, 3H), 8.44 (s, 1H), 12.96 (s, 1H), 13.02-13.76 (m, 1H); LCMS M/z 420 (M+H)⁺.

The requisite 4-{3-(3H-imidazo[4,5-b]pyridin-2-yl)-5-[(1S)-2-methoxy-1-methylethoxy]phenoxy}benzonitrile was prepared as follows:

### Methyl 3-(4-cyanophenoxy)-5-hydroxybenzoate

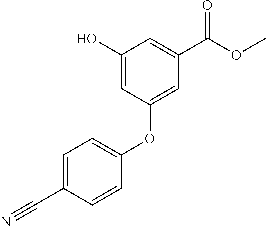

A solution of methyl 3,5 dihydroxy benzoate (50.4 g, 300 mmol) in DMA (220 ml) was treated with 4-fluorobenzonitrile (12.1 g, 100 mmol) and caesium carbonate (32.5 g, 100 mmol), and the resulting suspension heated at 120° C. under an argon atmosphere for 2 hrs. Most of the solvent was removed in vacuo, and the residue treated with water (500 ml); the aqueous mixture was washed twice with ethyl acetate, the washings combined, dried (MgSO₄) and evaporated to give ˜60 g of a brown pasty solid. This was redissolved in ethyl acetate and washed sequentially with 5×140 ml of 1M potassium carbonate solution, and the organic solution then dried (MgSO₄) and evaporated. The residue was purified by chromatography (400 g silica column, Biotage Flash 75™, gradient eluting with DCM containing 0%-20% ethyl acetate) to give the title compound (7.96 g, 88%). ¹H NMR (400 MHz, DMSO) δ, 3.86 (s, 3H), 6.77 (t, 1H), 6.99-7.05 (m, 1H), 7.12-7.20 (m, 2H), 7.22-7.27 (m, 1H), 7.82-7.91 (m, 2H), 10.35 (s, 1H), m/z 268 (M−H)⁻.

### Methyl 3-(4-cyanophenoxy)-5-[(1S)-2-methoxy-1-methylethoxy]benzoate

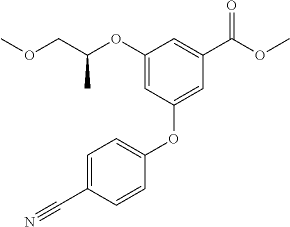

A solution of methyl 3-(4-cyanophenoxy)-5-hydroxybenzoate (5.0 g, 18.6 mmol), (R)-1-methoxy-2-propanol (2.72 ml, 27.9 mmol) and triphenyl phosphine (7.30 g, 27.9 mmol) in anhydrous THF (300 ml) was treated in an argon atmosphere with diethyl azodicarboxylate (DEAD, 4.39 ml, 27.9 mmol added dropwise). The temperature rose from 20° C. to 30° C.; the pale yellow solution was stirred for 2½ hours and then concentrated to a yellow oil. This was stirred in EtOAc/hexane (300 ml of a 1:1 mixture) over the weekend and then filtered and the filtrate concentrated to a yellow oil. This was purified by chromatography (400 g silica column, Biotage Flash 75™, gradient eluting with hexane containing 0%-30% ethyl acetate) to give the title compound (6.35 g, 100%). ¹H NMR (400 MHz, DMSO) δ 1.23 (d, 3H), 3.33 (s, 3H), 3.42-3.54 (m, 2H), 3.86 (s, 3H), 4.66-4.77 (m, 1H), 7.07 (t, 1H), 7.12-7.16 (m, 1H), 7.15-7.21 (m, 2H), 7.30-7.37 (m, 1H), 7.84-7.91 (m, 2H), m/z 340 (M−H)⁻.

### 3-(4-cyanophenoxy)-5-[(1S)-2-methoxy-1-methylethoxy]benzoic acid

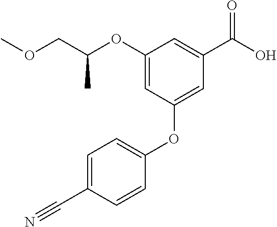

A solution of sodium hydroxide (1.5 g, 37.2 mmol) in water (100 ml) was added dropwise over 20 mins to a solution of methyl 3-(4-cyanophenoxy)-5-[(1S)-2-methoxy-1-methylethoxy]benzoate (6.35 g, 18.6 mmol) in THF (100 ml) and methanol (100 ml). The pale yellow solution was stirred at ambient temperature for 2 hours and then acidified using 2M HCl (excess). The organic solvent was evaporated in vacuo and the resulting watery oil was extracted with ethyl acetate (×3); the combined extracts were washed with brine, dried (MgSO₄) and concentrated to give the title compound as a pale yellow oil, ¹H NMR (400 MHz, DMSO) δ 1.23 (d, 3H), 3.33 (s, 3H), 3.43-3.53 (m, 2H), 4.65-4.74 (m, 1H), 7.02 (t, 1H), 7.10-7.12 (m, 1H), 7.15-7.21 (m, 2H), 7.31-7.35 (m, 1H), 7.83-7.91 (m, 2H), 13.41 (s, 1H), m/z 326, (M−H)⁻.

### 4-{3-(3H-imidazo[4,5-b]pyridin-2-yl)-5-[(1S)-2-methoxy-1-methylethoxy]phenoxy}benzonitrile

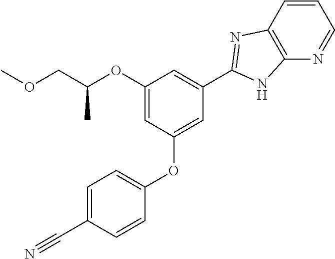

This was prepared in a manner essentially similar to that described for Example 3, starting from 3-(4-cyanophenoxy)-5-[(1S)-2-methoxy-1-methylethoxy]benzoic acid and 2,3 diamino pyridine, to give the title compound, ¹H NMR (400 MHz, DMSO) δ 1.29 (d, 3H), 3.30 (s, 3H), 3.54 (t, 2H), 4.73-4.83 (m, 1H), 6.95-6.98 (m, 1H), 7.26 (d, 2H), 7.31-7.38 (m, 1H), 7.49-7.54 (m, 1H), 7.71-7.75 (m, 1H), 7.91 (d, 2H), 8.13 (d, 1H), 8.42 (d, 1H), m/z 401, (M+H)⁺.

### EXAMPLE 63

### 2-{3-[(1S)-2-methoxy-1-methylethoxy]-5-[4-(pyrrolidin-1-ylcarbonyl)phenoxy]phenyl}-3H-imidazo[4,5-b]pyridine

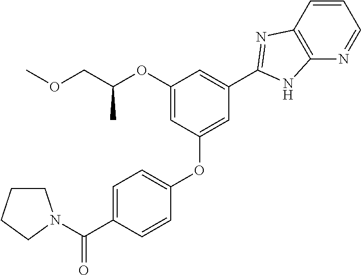

A solution of pyrrolidine (48 μL, 0.57 mmol), DIPEA (249 μL, 1.43 mmol) and 4-{3-(3H-imidazo[4,5-b]pyridin-2-yl)-5-[(1S)-2-methoxy-1-methylethoxy]phenoxy}benzoic acid (Example 62; 200 mg, 0.48 mmol) in anhydrous DMF (2 ml) was stirred at room temperature. for 5 mins. HATU (181 mg, 0.48 mmol) was then added and the orange solution stirred at ambient temperature for 2 days. The reaction mixture was concentrated and the resulting brown oil was dissolved in DCM and eluted sequentially through a 1 g Isolute™ SCX-2 acid scavenger column followed by a 1 g Isolute™ NH2 basic scavenger column. The resulting eluate was concentrated to give product (125 mg, approx. 90% purity).

This was purified by reverse phase preparative HPLC to give the title compound (66 mg, 29%). ¹H NMR (400 MHz, DMSO) δ 1.28 (d, 3H), 1.79-1.93 (m, 4H), 3.32 (s, 3H), 3.42-3.56 (m, 6H), 4.70 (sextet, 1H), 6.67 (t, 1H), 6.95 (dd, 1H), 7.11 (d, 2H), 7.50 (s, 1H), 7.59 (d, 2H), 7.69 (s, 1H), 7.77 (d, 1H), 8.12 (d, 1H), m/z 473 (M+H)⁺.

The following examples were made by a method essentially similar to that described for Example 63:

| Ex No | Structure | NMR | M/Z |
|---|---|---|---|
| 64 | 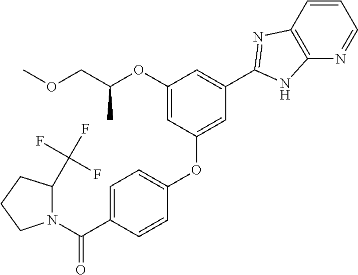 | ¹H NMR (500 MHz, DMSO-d₆) δ 1.33 (3H, d), 1.85-1.93 (1H, m), 1.94-2.08 (2H, m), 2.18-2.28 (1H, m), 3.34 (3H, s), , 3.52- 3.61 (4H, m), 4.71-4.74 (1H, m), 5.05 (1H, d), 6.81 (1H, t), 7.17-7.19 (2H, m), 7.19-7.23 (1H, m), 7.54-7.55 (1H, m), 7.62-7.64 (2H, m), 7.68-7.69 (1H, m), 7.95-7.97 (1H, m), 8.33-8.35 (1H, m); the spectrum also contains signals due to DMF (~30 mol %) | 541 (M + H)⁺ |
| 65 | 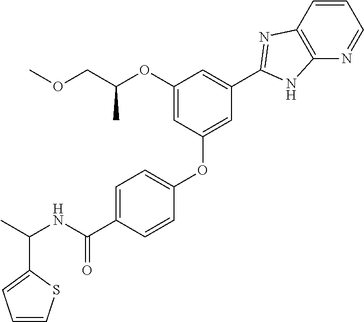 | ¹H NMR (500 MHz, DMSO-d₆) δ 1.33 (3H, d), 1.63 (3H, d), 3.34 (3H, s), 3.51-3.54 (1H, m), 3.57-3.61 (1H, m), 4.70-4.74 (1H, m), 5.48 (1H, t), 6.80 (1H, t), 6.97- 6.99 (1H, m), 7.04-7.05 (1H, m), 7.16- 7.19 (2H, m), 7.21-7.24 (1H, m), 7.32- 7.34 (1H, m), 7.51-7.52 (1H, m), 7.66- 7.67 (1H, m), 7.96-7.98 (3H, m), 8.34- 8.35 (1H, m), 8.51 (1H, d) | 529 (M + H)⁺ |
| 66 | 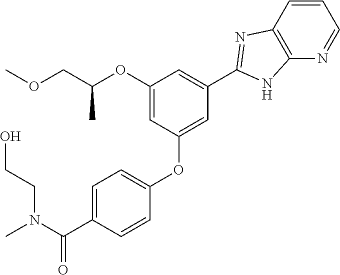 | ¹H NMR (500 MHz, DMSO-d₆) δ 1.33 (3H, d), 3.03 (3H, s), 3.47 (2H, t), 3.51-3.54 (1H, m), 3.58 (1H, d), 3.34 (3H, s), 3.63 (2H, t), 4.26-4.47 (1H, bs), 4.70-4.73 (1H, m), 6.80 (1H, t), 7.13-7.16 (2H, m), 7.21- 7.24 (1H, m), 7.47-7.50 (2H, m), 7.54 (1H, t), 7.67 (1H, t), 7.96-7.98 (1H, m), 8.34-8.35 (1H, m); the spectrum also contains signals due to DMF (~100 mol %) | 477 (M + H)⁺ |
| 67 | 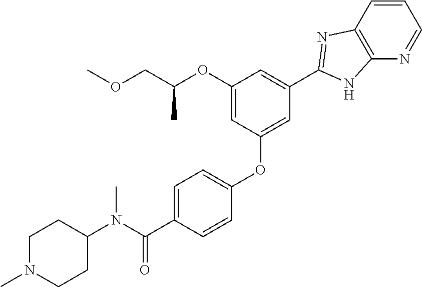 | ¹H NMR (500 MHz, DMSO-d₆) δ 1.33 (3H, d), 1.92 (2H, d), 2.12 (2H, d), 2.79 (3H, s), 2.89 (3H, s), 3.07 (2H, d), 3.33 (3H, s), 3.46 (2H, d), 3.52-3.55 (1H, m), 3.58- 3.61 (1H, m), 4.14-4.28 (1H, m), 4.71- 4.74 (1H, m), 6.81 (1H, t), 7.17-7.18 (2H, m), 7.23-7.25 (1H, m), 7.47-7.49 (2H, m), 7.53 (1H, s), 7.67 (1H, s), 7.97 (1H, d), 8.35 (1H, d) | 530 (M + H)⁺ |
| 68 | 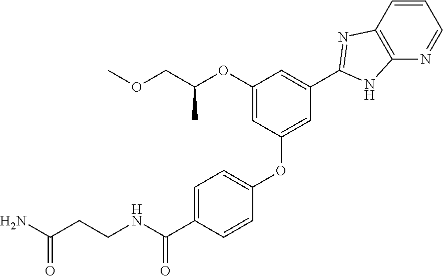 | ¹H NMR (500 MHz, DMSO-d₆) δ 1.33 (3H, d), 2.42 (2H, t), 3.34 (3H, s), 3.50-3.54 (3H, m), 3.57-3.61 (1H, m), 4.70-4.74 (1H, m), 6.55-6.77 (2H, br s, integrates low — assigned as —NH₂) 6.80 (1H, t), 7.15- 7.18 (211, m), 7.22-7.24 (1H, m), 7.51 (1H, t), 7.67 (1H, t), 7.89-7.92 (2H, m), 7.97 (1H, d), 8.08 (1H, s), 8.34-8.36 (1H, m) | 490 (M + H)⁺ |
| 69 | 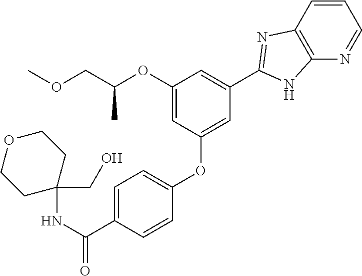 | ¹H NMR (500 MHz, DMSO-d₆) δ 1.33 (3H, d), 1.69-1.75 (2H, m), 2.23-2.25 (2H, m), 3.34 (3H, s), 3.52-3.55 (1H, m), 3.58- 3.61 (1H, m), 3.65-3.70 (6H, m), 4.34- 4.54 (1H, BS), 4.70-4.74 (1H, m), 6.79 (1H, t), 7.15-7.17 (211, m), 7.16-7.21 (1H, m), 7.26 (1H, s), 7.49 (114, s), 7.66- 7.67 (1H, m), 7.90-7.98 (311, m), 8.32- 8.34 (1H, m); the spectrum also contains signals due to DMF (~100 mol %) | 533 (M + H)⁺ |

MS and retention time (RT) data for the following examples were generated using the following method:

### Liquid Chromatography-Mass Spectrometry (LC-MS) Accurate Mass Method.

Liquid Chromatography-Mass Spectrometry analysis was carried out on a ‘time-of-flight’ mass spectrometer system (Waters LCT MS) fitted with a High Performance Liquid Chromatography (HPLC) system consisting of a binary pump, an autosampler and a diode array detector (DAD) (Agilent HP1100). Both systems were controlled by Water's MassLynx software, version 4.0 (Waters Ltd, Manchester, UK). The Liquid Chromatography column was a Gemini 50 mm, 2.1 mm i.d. with 5 um particles (Phenomenex, Macclesfield, UK). The sample was dissolved in 50:50 (v/v) water/acetonitrile at ˜0.1 μg/ml concentration and 50 injected. The mobile phase was set at a flow rate of 0.5 ml/min. Mobile phase A was 0.05% aqueous formic acid and mobile phase B was 0.05% formic acid in acetonitrile. The following HPLC gradient was used:

| Time/min | % A | % B |
|---|---|---|
| 0.00 | 95 | 5 |
| 2.00 | 95 | 5 |
| 13.0 | 0 | 100 |
| 15.8 | 0 | 100 |
| 16.0 | 95 | 5 |
| 20.0 | 95 | 5 |

The DAD scanned from 200 to 300 nm at 2 nm steps in 0.5 secs.

The mass spectrometer used electrospray ionisation (ESI) in either positive or negative ion mode. The capillary voltage was 600 V and 3500 V in positive and negative ion respectively. The desolvation gas flow was set to 1 L/hr, the source temperature to 120° C. and the desolvation temperature to 350° C. A lock mass was used to ensure accurate mass measurement. This was m/z 409.1854 in positive ion and m/z 406.1667 in negative ion. The lock mass sample was infused via a separate electrospray probe and sampled every 5 sec. All data was collected as centroided spectra.

For LC-MS mass spectra the instrument scanned the mass range 100 to 1000 amu at a scan speed at 0.5 sec/scan with a 0.1 sec scan interval. The cone voltage was set to 25 eV.

For LC-MS fragment ion spectra the instrument scanned the mass range 100 to 1000 amu at a scan speed at 0.5 sec/scan with a 0.1 sec scan interval using sequential source cone voltages of 25, 50 and 80 eV.

| | | M/z | |
|---|---|---|---|
| Ex No | Structure | (M + H)⁺ | RT (min) |
| 70 | 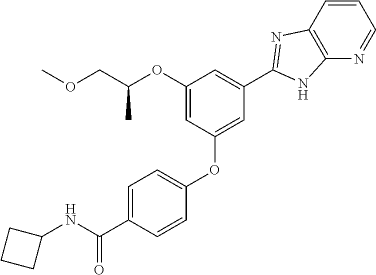 | 473 | 7.68 |
| 71 | 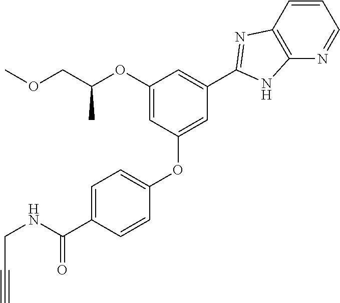 | 457 | 7.2 |
| 72 | 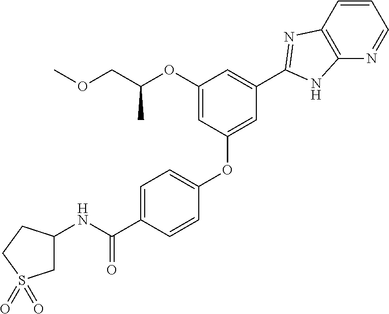 | 537 | 6.92 |
| 73 | 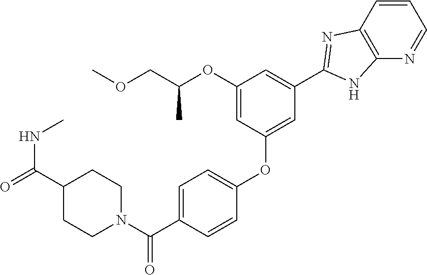 | 544 | 6.54 |
| 74 | 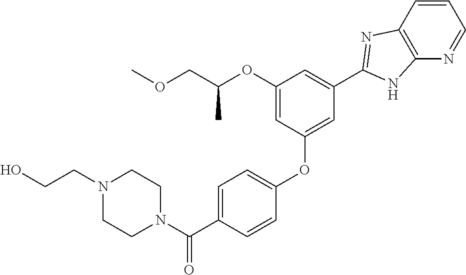 | 532 | 5.21 |
| 75 | 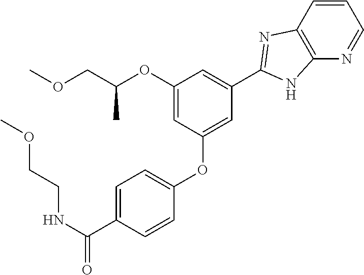 | 477 | 6.87 |
| 76 | 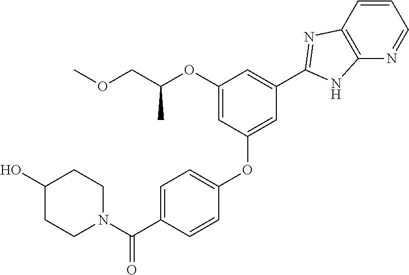 | 503 | 6.53 |
| 77 | 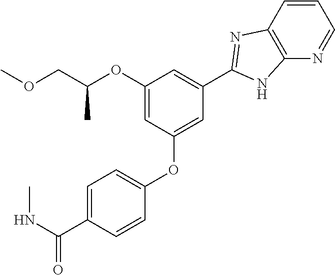 | 433 | 6.66 |
| 78 | 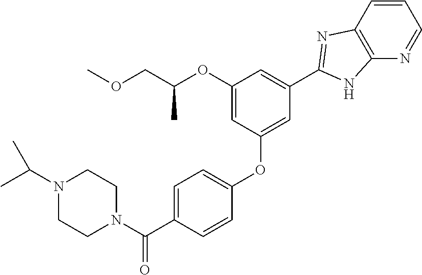 | 530 | 5.42 |
| 79 | 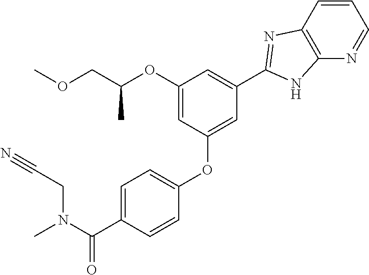 | 472 | 7.22 |
| 80 | 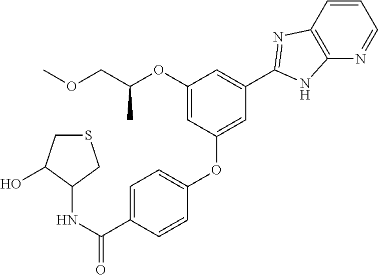 | 521 | 7.01 |
| 81 | 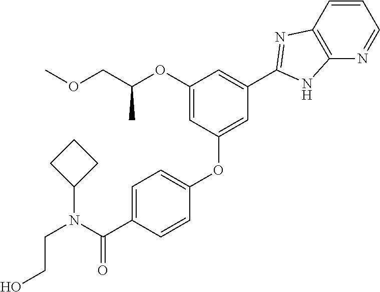 | 517 | 7.33 |
| 82 | 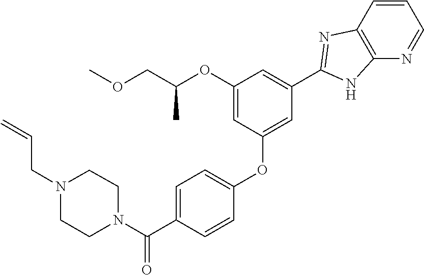 | 528 | 5.46 |
| 83 | 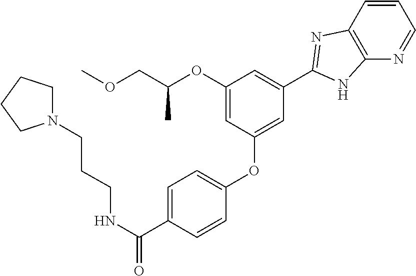 | 530 | 5.53 |
| 84 | 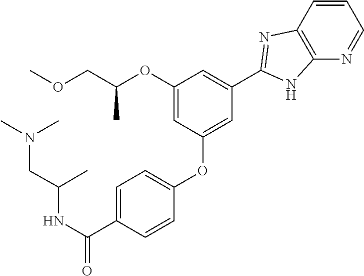 | 504 | 5.48 |

### EXAMPLE 64

2-[3-[(1S)-2-methoxy-1-methylethoxy]-5-(4-{[2-(trifluoromethyl)pyrrolidin-1-yl]carbonyl}phenoxy)phenyl]-3H-imidazo[4,5-b]pyridine;

### EXAMPLE 65

4-{3-(3H-imidazo[4,5-b]pyridin-2-yl)-5-[(1S)-2-methoxy-1-methylethoxy]phenoxy}-N-[1-(2-thielayl)ethyl]benzamide;

### EXAMPLE 66

N-(2-hydroxyethyl)-4-{3-(3H-imidazo[4,5-b]pyridin-2-yl)-5-[(1S)-2-methoxy-1-methylethoxy]phenoxy}-N-methylbenzamide;

### EXAMPLE 67

4-{3-(3H-imidazo[4,5-b]pyridin-2-yl)-5-[(1S)-2-methoxy-1-methylethoxy]phenoxy}-N-methyl-N-(1-methylpiperidin-4-yl)benzamide;

### EXAMPLE 68

N-(3-amino-3-oxopropyl)-4-{3-(3H-imidazo[4,5-b]pyridin-2-yl)-5-[(1S)-2-methoxy-1-methylethoxy]phenoxy}benzamide;

### EXAMPLE 69

N-[4-(hydroxymethyl)tetrahydro-2H-pyran-4-yl]-4-{3-(3H-imidazo[4,5-b]pyridin-2-yl)-5-[(1S)-2-methoxy-1-methylethoxy]phenoxy}benzamide;

### EXAMPLE 70

N-cyclobutyl-4-{3-(3H-imidazo[4,5-b]pyridin-2-yl)-5-[(1S)-2-methoxy-1-methylethoxy]phenoxy}benzamide;

### EXAMPLE 71

4-{3-(3H-imidazo[4,5-b]pyridin-2-yl)-5-[(1S)-2-methoxy-1-methylethoxy]phenoxy}-N-prop-2-yn-1-ylbenzamide;

### EXAMPLE 72

N-(1,1-dioxidotetrahydro-3-thienyl)-4-{3-(3H-imidazo[4,5-b]pyridin-2-yl)-5-[(1S)-2-methoxy-1-methylethoxy]phenoxy}benzamide;

### EXAMPLE 73

1-(4-{3-(3H-imidazo[4,5-b]pyridin-2-yl)-5-[(1S)-2-methoxy-1-methylethoxy]phenoxy}benzoyl)-N-methylpiperidine-4-carboxamide;

### EXAMPLE 74

2-[4-(4-{3-(3H-imidazo[4,5-b]pyridin-2-yl)-5-[(1S)-2-methoxy-1-methylethoxy]phenoxy}benzoyl)piperazin-1-yl]ethanol;

### EXAMPLE 75

4-{3-(3H-imidazo[4,5-b]pyridin-2-yl)-5-[(1S)-2-methoxy-1-methylethoxy]phenoxy}-N-(2-methoxyethyl)benzamide;

### EXAMPLE 76

1-(4-{3-(3H-imidazo[4,5-b]pyridin-2-yl)-5-[(1S)-2-methoxy-1-methylethoxy]phenoxy}benzoyl)piperidin-4-ol;

### EXAMPLE 77

4-{3-(3H-imidazo[4,5-b]pyridin-2-yl)-5-[(1S)-2-methoxy-1-methylethoxy]phenoxy}-N-methylbenzamide;

### EXAMPLE 78

2-{3-{4-[(4-isopropylpiperazin-1-yl)carbonyl]phenoxy}-5-[(1S)-2-methoxy-1-methylethoxy]phenyl}-3H-imidazo[4,5-b]pyridine;

### EXAMPLE 79

N-(cyanomethyl)-4-{3-(3H-imidazo[4,5-b]pyridin-2-yl)-5-[(1S)-2-methoxy-1-methylethoxy]phenoxy}-N-methylbenzamide;

### EXAMPLE 80

N-(4-hydroxytetrahydro-3-thienyl)-4-{3-(3H-imidazo[4,5-b]pyridin-2-yl)-5-[(1S)-2-methoxy-1-methylethoxy]phenoxy}benzamide;

### EXAMPLE 81

N-cyclobutyl-N-(2-hydroxyethyl)-4-{3-(3H-imidazo[4,5-b]pyridin-2-yl)-5-[(1S)-2-methoxy-1-methylethoxy]phenoxy}benzamide;

### EXAMPLE 82

2-{3-{4-[(4-allylpiperazin-1-yl)carbonyl]phenoxy}-5-[(1S)-2-methoxy-1-methylethoxy]phenyl}-3H-imidazo[4,5-b]pyridine;

### EXAMPLE 83

4-{3-(3H-imidazo[4,5-b]pyridin-2-yl)-5-[(1S)-2-methoxy-1-methylethoxy]phenoxy}-N-(3-pyrrolidin-1-ylpropyl)benzamide;

### EXAMPLE 84

N-[2-(dimethylamino)-1-methylethyl]-4-{3-(3H-imidazo[4,5-b]pyridin-2-yl)-5-[(1S)-2-methoxy-1-methylethoxy]phenoxy}benzamide.

The following examples were made by a similar method to Examples 64-69:

| Ex No | Structure | NMR | M/Z |
|---|---|---|---|
| 85 | 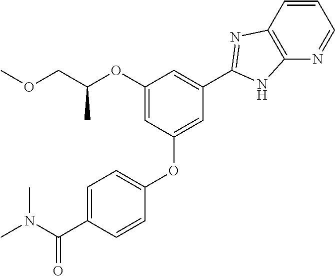 | ¹H NMR (400 MHz, DMSO-d₆₊CD₃CO₂D) δ 1.29 (3H, d), 2.98 (6H, s), 3.31 (6H, s) 3.48-3.57 (2H, m), 4.73-4.77 (1H, m), 6.82 (1H, t), 7.13-7.16 (2H, m), 7.22- 7.26 (1H, m), 7.47-7.50 (3H, m), 7.65 (1H, t), 8.00 (1H, s), 8.34-8.35 (1H, m), 11.91 (5H, s) | 447 (M + H)⁺ |
| 86 | 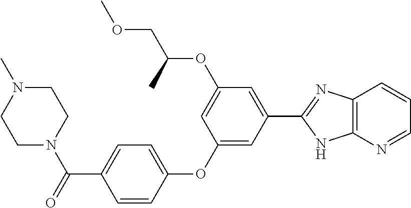 | ¹H NMR (400 MHz, DMSO-d₆ + CD₃CO₂D) δ 1.29 (3H, d), 2.33 (3H, s), 2.50-2.56 (4H, m), 3.31 (3H, s), 3.48- 3.58 (6H, m), 4.73-4.77 (1H, m), 6.83 (1H, t), 7.14-7.17 (2H, m), 7.23-7.26 (1H, m), 7.47-7.50 (3H, m), 7.66 (1H, t), 8.00 (1H, d), 8.34-8.35 (1H, m) | 500 (M − H)⁻ 502 (M + H)⁺ |

### EXAMPLE 85

4-{3-(3H-imidazo[4,5-b]pyridin-2-yl)-5-[(1S)-2-methoxy-1-methylethoxy]phenoxy}-N,N-dimethylbenzamide

### EXAMPLE 86

2-(3-[(1S)-2-methoxy-1-methylethoxy]-5-{4-[(4-methylpiperazin-1-yl)carbonyl]phenoxy}phenyl)-3H-imidazo[4,5-b]pyridine

### EXAMPLE 87

### 2-{3-[(1S)-2-methoxy-1-methylethoxy]-5-[4-(methylsulfonyl)phenoxy]phenyl}-1H-imidazo[4,5-b]pyrazine

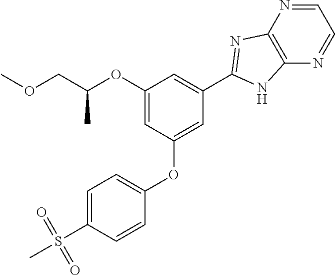

A solution of 5-bromo-2-{3-[(1S)-2-methoxy-1-methylethoxy]-5-[4-(methylsulfonyl)phenoxy]phenyl}-1H-imidazo[4,5-b]pyrazine (100 mg, 0.19 mmol) in a mixture of EtOH and MeOH (7.5 ml of 1:1) was treated with a catalytic amount of palladium on charcoal (50 mg of 10% Pd)) under an atmosphere of hydrogen overnight. The reaction mixture was filtered and the filtrate evaporated in vacuo. The resultant solid was then purified by preparative HPLC to give the title compound as a beige solid, 31 mg, ¹H NMR (400 MHz, DMSO) δ 1.30 (d, 3H), 3.23 (s, 3H) 3.26-3.38 (s, 3H+H₂O), 3.46-3.60 (m, 2H), 4.76-4.85 (m, 1H), 6.98-7.03 (m, 1H), 7.29-7.36 (m, 2H), 7.58-7.61 (m, 1H), 7.75-7.80 (m, 1H), 7.95-8.01 (m, 2H), 8.33-8.52 (m, 2H), 13.94 (s, 1H), m/z 453 (M−H)⁻.

### EXAMPLE 88

### 2-{3-{[6-(Azetidin-1-ylcarbonyl)pyridin-3-yl]oxy}-5-[(3S)-tetrahydrofuran-3-yloxy]phenyl}-6-fluoro-3H-imidazo[4,5-b]pyridine

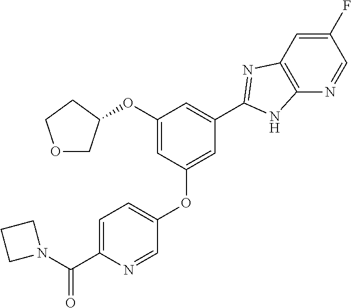

1,1-Carbonyldiimide (214 mg, 1.32 mmol) was added to a solution of 3-{[6-(azetidin-1-ylcarbonyl)pyridin-3-yl]oxy}-5-[(3S)-tetrahydrofuran-3-yloxy]benzoic acid (423 mg, 1.00 mmol) in butyronitrile (5.0 mL). The solution was stirred at RT for 5 minutes before the 2,3-diamino-5-fluoropyridine (140 mg, 1.00 mmol) was added. The reaction was heated in a microwave reactor for 2 hours at 185° C., the black residue dissolved in a little DCM and MeOH and chromatographed on alumina, eluting with 0-50% ethylacetate in isohexane followed by elution with 0-10% MeOH in DCM, to give the desired compound as a white solid (35 mg). ¹H NMR (400 MHz, DMSO) δ 2.35 (m, 4H), 3.85 (m, 1H), 3.93 (m, 2H), 3.99 (m, 1H), 4.14 (t, 2H), 4.66 (t, 2H), 5.23 (s, 1H), 6.99 (m, 1H), 7.56 (s, 1H), 7.70 (m, 2H), 8.09 (s, 2H), 8.41 (m, 1H), 8.53 (d, 1H), 13.78 (s, 1H); m/z 474 (M−H)⁻

The preparation of 3-{[6-(azetidin-1-ylcarbonyl)pyridin-3-yl]oxy}-5-[(3S)-tetrahydrofuran-3-yloxy]benzoic acid was described earlier.

### EXAMPLE 89

### N,N-Dimethyl-5-({3-(9H-purin-8-yl)-5-[(3S)-tetrahydrofuran-3-yloxy]phenyl}oxy)pyrazine-2-carboxamide

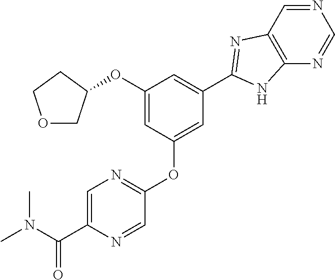

A mixture of 3-(9H-purin-8-yl)-5-[(3S)-tetrahydrofuran-3-yloxy]phenol (100 mg, 0.34 mmol), 5-chloro-N,N-dimethylpyrazine-2-carboxamide (75 mg, 0.40 mmol) and potassium carbonate (93 mg, 0.67 mmol) was heated for 2 hours at 120° C. in microwave reactor. The mixture was filtered and concentrated in vacuo. The residue was dissolved in the minimum DCM and chromatographed on silica, eluting with 0-10% MeOH in ethylacetate, then re-chromatographed on alumina to give the desired compound as a pale yellow solid (60 mg). ¹N NMR (400 MHz, DMSO) δ 2.07-2.15 (m, 1H), 2.32-2.42 (m, 1H), 3.10 (s, 3H), 3.10 (s, 3H), 3.82-3.89 (m, 1H), 3.91-3.98 (m, 2H), 3.98-4.03 (m, 1H), 5.24 (m, 1H), 7.18 (s, 1H), 7.78 (t, 2H), 8.51 (d, 1H), 8.66 (d, 1H), 8.97 (s, 1H), 9.18 (s, 1H), 14.03 (s, 1H); m/z 448 (M+H)⁺

The preparation of 5-chloro-N,N-dimethylpyrazine-2-carboxamide was described earlier.

The preparation of 3-(9H-purin-8-yl)-5-[(3S)-tetrahydrofuran-3-yloxy]phenol is described below.

### 3-(9H-Purin-8-yl)-5-[(3S)-tetrahydrofuran-3-yloxy]phenol

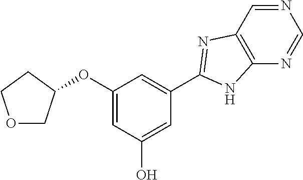

Palladium on charcoal (10% by wt, 107 mg) was added to a mixture of 8-{3-[(phenylmethyl)oxy]-5-[(3S)-tetrahydrofuran-3-yloxy]phenyl}-9H-purine (500 mg, 1.29 mmol) in MeOH (25 mL) and the mixture stirred at RT under a hydrogen atmosphere for 3 days. The catalyst removed by filtration and fresh catalyst added. The mixture was stirred for a further 16 hours under a hydrogen atmosphere then the catalyst removed by filtration and the filtrate concentrated in vacuo. The residue was triturated with ethanol to give the desired compound as a cream coloured solid (231 mg). m/z 299 (M+H)⁺

### 8-{3-[(Phenylmethyl)oxy]-5-[(3S)-tetrahydrofuran-3-yloxy]phenyl}-9H-purine

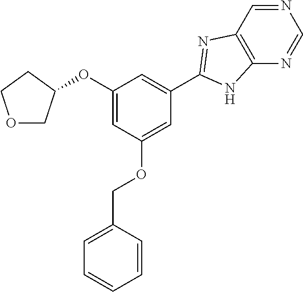

1,1-Carbodiimide (620 mg, 3.82 mmol) was added to a solution of 3-[(phenylmethyl)oxy]-5-[(3S)-tetrahydrofuran-3-yloxy]benzoic acid (1000 mg, 3.18 mmol) in butyronitrile (10 mL). The solution was stirred at RT then 4,5-diaminopyrimidine (351 mg, 3.18 mmol) added and the mixture stirred at 200° C. for 2 hours in microwave reactor. The mixture was reduced in vacuo, the residue triturated with MeOH (20 mL) and filtered to give the desired compound as a cream coloured solid (548 mg). ¹H NMR (400 MHz, DMSO) δ 1.95-2.05 (m, 1H), 2.20-2.35 (m, 1H), 3.70-4.00 (m, 4H), 5.15 (m, 1H), 5.22 (s, 2H), 6.79 (s, 1H), 7.30-7.60 (m, 7H), 8.91 (s, 1H), 9.11 (s, 1H), 13.82 (brs, 1H); m/z 389 (M+H)⁺, 387 (M−H)⁻

### 3-[(Phenylmethyl)oxy]-5-[(3S)-tetrahydrofuran-3-yloxy]benzoic acid

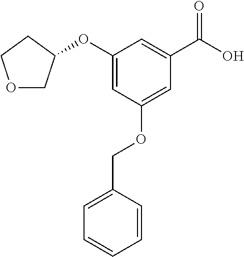

Lithium hydroxide monohydrate (1.94 g, 46.14 mmol) in water (80 mL) was added to methyl 3-[(phenylmethyl)oxy]-5-[(3S)-tetrahydrofuran-3-yloxy]benzoate (10.1 g, 30.76 mmol) in THF (160 mL) and the mixture stirred at RT for 72 hours. The organics were removed in vacuo and the aqueous residue adjusted to pH3 with citric acid. The product was extracted into ethyl actetate and the organic phase washed with water (30 mL), brine (30 mL), dried (MgSO₄), filtered and the solvent removed in vacuo to give the desired compound as a white solid (8 g). ¹H NMR (400 MHz, DMSO) δ 1.92-1.98 (m, 1H), 2.17-2.26 (m, 1H), 3.72-3.89 (m, 4H), 5.07-5.09 (m, 1H), 5.15 (s, 2H), 6.81 (t, 1H), 7.03 (s, 1H), 7.15 (s, 1H), 7.32-7.47 (m, 5H), 13.07 (s, 1H); m/z 315 (M+H)⁺

The preparation of methyl 3-[(phenylmethyl)oxy]-5-[(3S)-tetrahydrofuran-3-yloxy]benzoate was described earlier.

### Biological

### Tests:

The biological effects of the compounds of formula (I) may be tested in the following way:

### (1) Enzymatic Activity

Enzymatic activity of recombinant human pancreatic GLK may be measured by incubating GLK, ATP and glucose. The rate of product formation may be determined by coupling the assay to a G-6-P dehydrogenase, NADP/NADPH system and measuring the linear increase with time of optical density at 340 nm (Matschinsky et al 1993). Activation of GLK by compounds can be assessed using this assay in the presence or absence of GLKRP as described in Brocklehurst et al (Diabetes 2004, 53, 535-541).

### Production of Recombinant GLK and GLKRP:

Human GLK and GLKRP cDNA was obtained by PCR from human pancreatic and hepatic mRNA respectively, using established techniques described in Sambrook J, Fritsch EF & Maniatis T, 1989. PCR primers were designed according to the GLK and GLKRP cDNA sequences shown in Tanizawa et al 1991 and Bonthron, D. T. et al 1994 (later corrected in Warner, J. P. 1995).

### Cloning in Bluescript II Vectors

GLK and GLKRP cDNA was cloned in E. coli using pBluescript II, (Short et al 1998) a recombinant cloning vector system similar to that employed by Yanisch-Perron C et al (1985), comprising a colEI-based replicon bearing a polylinker DNA fragment containing multiple unique restriction sites, flanked by bacteriophage T3 and T7 promoter sequences; a filamentous phage origin of replication and an ampicillin drug resistance marker gene.

### Transformations

E. Coli transformations were generally carried out by electroporation. 400 mL cultures of strains DH5a or BL21(DE3) were grown in L-broth to an OD 600 of 0.5 and harvested by centrifugation at 2,000 g. The cells were washed twice in ice-cold deionised water, resuspended in 1 mL 10% glycerol and stored in aliquots at −70° C. Ligation mixes were desalted using Millipore V Series™ membranes (0.0025 mm) pore size). 40 mL of cells were incubated with 1 mL of ligation mix or plasmid DNA on ice for 10 minutes in 0.2 cm electroporation cuvettes, and then pulsed using a Gene Pulser™ apparatus (BioRad) at 0.5 kVcm⁻¹, 250 mF. Transformants were selected on L-agar supplemented with tetracyline at 10 mg/mL or ampicillin at 100 mg/mL.

### Expression

GLK was expressed from the vector pTB375NBSE in E. coli BL21 cells, producing a recombinant protein containing a 6-His tag immediately adjacent to the N-terminal methionine. Alternatively, another suitable vector is pET21(+)DNA, Novagen, Cat number 697703. The 6-His tag was used to allow purification of the recombinant protein on a column packed with nickel-nitrilotriacetic acid agarose purchased from Qiagen (cat no 30250).

GLKRP was expressed from the vector pFLAG CTC (IBI Kodak) in E. coli BL21 cells, producing a recombinant protein containing a C-terminal FLAG tag. The protein was purified initially by DEAE Sepharose ion exchange followed by utilisation of the FLAG tag for final purification on an M2 anti-FLAG immunoaffinity column purchased from Sigma-Aldrich (cat no. Al205).

Compounds of the invention generally have an activating activity for glucokinase with an EC₅₀ of less than about 30 μM, particularly less than about 10 μM, preferably less than about 1 μM, more preferably less than about 0.1 μM. For example, Example 1 has an EC₅₀ of 0.6 μM

### REFERENCES

1 Printz, R. L., Magnuson, M. A. and Granner, D. K. (1993) Annual Review of Nutrition 13, 463-96
2 DeFronzo, R. A. (1988) Diabetes 37, 667-87
3 Froguel, P., Zouali, H., Vionnet, N., Velho, G., Vaxillaire, M., Sun, F., Lesage, S., Stoffel, M., Takeda, J. and Passa, P. (1993) New England Journal of Medicine 328, 697-702
4 Bell, G. I., Pilkis, S. J., Weber, I. T. and Polonsky, K. S. (1996) Annual Review of Physiology 58, 171-86
5 Velho, G., Petersen, K. F., Perseghin, G., Hwang, J. H., Rothman, D. L., Pueyo, M. E., Cline, G. W., Froguel, P. and Shulman, G. I. (1996) Journal of Clinical Investigation 98, 1755-61
6 Christesen, H. B., Jacobsen, B. B., Odili, S., Buettger, C., Cuesta-Munoz, A., Hansen, T., Brusgaard, K., Massa, O., Magnuson, M. A., Shiota, C., Matschinsky, F. M. and Barbetti, F. (2002) Diabetes 51, 1240-6
6a Gloyn, A. L., Noordam, K., Willemsen, M. A. A. P., Ellard, S., Lam, W. W. K., Campbell, I. W., Midgley, P., Shiota, C., Buettger, C., Magnuson, M. A., Matschinsky, F. M., and Hattersley, A. T.; Diabetes 52: 2433-2440
7 Glaser, B., Kesavan, P., Heyman, M., Davis, E., Cuesta, A., Buchs, A., Stanley, C. A., Thornton, P. S., Permutt, M. A., Matschinsky, F. M. and Herold, K. C. (1998) New England Journal of Medicine 338, 226-30
8 Caro, J. F., Triester, S., Patel, V. K., Tapscott, E. B., Frazier, N. L. and Dohm, G. L. (1995) Hormone & Metabolic Research 27, 19-22
9 Desai, U. J., Slosberg, E. D., Boettcher, B. R., Caplan, S. L., Fanelli, B., Stephan, Z., Gunther, V. J., Kaleko, M. and Connelly, S. (2001) Diabetes 50, 2287-95
10 Shiota, M., Postic, C., Fujimoto, Y., Jetton, T. L., Dixon, K., Pan, D., Grimsby, J., Grippo, J. F., Magnuson, M. A. and Chemington, A. D. (2001) Diabetes 50, 622-9
11 Ferre, T., Pujol, A., Riu, E., Bosch, F. and Valera, A. (1996) Proceedings of the National Academy of Sciences of the United States of America 93, 7225-30
12 Seoane, J., Barbera, A., Telemaque-Potts, S., Newgard, C. B. and Guinovart, J. J. (1999) Journal of Biological Chemistry 274, 31833-8
13 Moore, M. C., Davis, S, N., Mann, S. L. and Chemington, A. D. (2001) Diabetes Care 24, 1882-7
14 Alvarez, E., Roncero, I., Chowen, J. A., Vazquez, P. and Blazquez, E. (2002) Journal of Neurochemistry 80, 45-53
15 Lynch, R. M., Tompkins, L. S., Brooks, H. L., Dunn-Meynell, A. A. and Levin, B. E. (2000) Diabetes 49, 693-700
16 Roncero, I., Alvarez, E., Vazquez, P. and Blazquez, E. (2000) Journal of Neurochemistry 74, 1848-57
17 Yang, X. J., Kow, L. M., Funabashi, T. and Mobbs, C. V. (1999) Diabetes 48, 1763-1772
18 Schuit, F. C., Huypens, P., Heimberg, H. and Pipeleers, D. G. (2001) Diabetes 50, 1-11
19 Levin, B. E. (2001) International Journal of Obesity 25, supplement 5, S68-S72.
20 Alvarez, E., Roncero, I., Chowen, J. A., Thorens, B. and Blazquez, E. (1996) Journal of Neurochemistry 66, 920-7
21 Mobbs, C. V., Kow, L. M. and Yang, X. J. (2001) American Journal of Physiology—Endocrinology & Metabolism 281, E649-54
22 Levin, B. E., Dunn-Meynell, A. A. and Routh, V. H. (1999) American Journal of Physiology 276, R1223-31
23 Spanswick, D., Smith, M. A., Groppi, V. E., Logan, S. D. and Ashford, M. L. (1997) Nature 390, 521-5
24 Spanswick, D., Smith, M. A., Mirshamsi, S., Routh, V. H. and Ashford, M. L. (2000) Nature Neuroscience 3, 757-8
25 Levin, B. E. and Dunn-Meynell, A. A. (1997) Brain Research 776, 146-53
26 Levin, B. E., Govek, E. K. and Dunn-Meynell, A. A. (1998) Brain Research 808, 317-9
27 Levin, B. E., Brown, K. L. and Dunn-Meynell, A. A. (1996) Brain Research 739, 293-300
28 Rowe, I. C., Boden, P. R. and Ashford, M. L. (1996) Journal of Physiology 497, 365-77
29 Fujimoto, K., Sokoto, T., Arase, K., Kurata, K., Okabe, Y. and Shiraishi, T. (1985) Life Sciences 37, 2475-82
30 Kurata, K., Fujimoto, K. and Sakata, T. (1989) Metabolism: Clinical & Experimental 38, 46-51
31 Kurata, K., Fujimoto, K., Sakata, T., Etou, H. and Fukagawa, K. (1986) Physiology & Behavior 37, 615-20
32 Jetton T. L., Liang Y., Pettepher C. C., Zimmerman E. C., Cox F. G., Horvath K., Matschinsky F. M., and Magnuson M. A., J. Biol. Chem., February 1994; 269: 3641-3654
33 Reimann F. and Gribble F. M., Diabetes 2002 51: 2757-2763
34 Cheung A. T., Dayanandan B., Lewis J. T., Korbutt G. S., Rajotte R. V., Bryer-Ash M., Boylan M. O., Wolfe M. M., Kieffer T. J., Science, Vol 290, Issue 5498, 1959-1962, 8 December 2000

1. A compound of Formula (I): 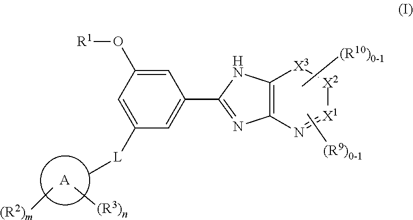
wherein:
Ring A is selected from phenyl and HET-1;
X¹, X² and X³ are each independently CH or N, with the proviso that only one of X¹, X² and X³ may be N;
L is a linker selected from —O— and -(1-3C)alkylO- wherein the oxygen is directly attached to the benzene ring which is substituted by —OR;
R¹ is selected from (1-6C)alkyl, (2-6C)alkenyl, (2-6C)alkynyl, (3-6C)cycloalkyl, (3-6C)cycloalkyl(1-6C)alkyl, aryl(1-6C)alkyl, HET-1 and HET-1-(1-6C)alkyl;
wherein any alkyl, alkenyl, alkynyl, cycloalkyl, aryl or HET-1 group in any definition of R¹ may optionally be substituted on an available carbon atom with a substituent selected from hydroxy, (1-4C)alkoxy, halo, (1-6C)alkylamino, di(1-6C)alkylamino, (CₙH₂ₙ₊₂₋ₐFₐ)—O— (wherein n=1 to 4 and a=1 to 3), (1-6C)alkylsulfonyl, (1-6C)alkylsulfonylamino, (1-6C)alkylsulfonyl-N-[(1-6C)alkyl]amino, (1-6C)alkylaminosulfonyl, di(1-6C)alkylaminosulfonyl, (1-6C)alkylcarbonylamino, (1-6C)alkylcarbonyl-N-[(1-6C)alkyl]amino, (1-6C)alkylaminocarbonyl, di(1-6C)alkylaminocarbonyl, carboxy and cyano; and/or substituted on an available nitrogen atom with a substituent selected from (1-6C)alkylsulfonyl, (1-6C)alkylaminosulfonyl, di(1-6C)alkylaminosulfonyl, (1-6C)alkylaminocarbonyl and di(1-6C)alkylaminocarbonyl;
HET-1 is a 4-, 5- or 6-membered, C- or N-linked saturated, partially or fully unsaturated heterocyclyl ring containing 1, 2, 3 or 4 heteroatoms independently selected from O, N and S, wherein a —CH₁— group can optionally be replaced by a —C(O)—, and wherein a sulphur atom in the heterocyclic ring may optionally be oxidised to a S(O) or S(O)₂ group;
R² is selected from —C(O)NR⁴R⁵, —SO₂NR⁴R⁵, —S(O)ₚR⁴ and HET-2;
HET-2 is a 4-, 5- or 6-membered, C- or N-linked saturated, partially or fully unsaturated heterocyclyl ring containing 1, 2, 3 or 4 heteroatoms independently selected from O, N and S, wherein a —CH₁— group can optionally be replaced by a —C(O)—, and wherein a sulphur atom in the heterocyclic ring may optionally be oxidised to a S(O) or S(O)₂ group, which ring is optionally substituted on an available nitrogen atom by a substituent selected from R⁶ and/or on an available carbon atom by 1 or 2 substituents independently selected from R⁷;
each R³ is independently selected from halo, fluoromethyl, difluoromethyl, trifluoromethyl, methyl, (1-4C)alkoxy, carboxy and cyano;
R⁴ is selected from hydrogen, (1-4C)alkyl (optionally substituted by 1 or 2 substituents independently selected from HET-2, —OR⁵, —SO₂R⁵, (3-6C)cycloalkyl (optionally substituted with 1 group selected from R⁷), cyano, —NR^{4′}R^{5′} and —C(O)NR⁵R⁵, (3-6C)cycloalkyl (optionally substituted with 1 group selected from R⁷), (2-4C)alkenyl (optionally substituted with 1 group selected from R⁷), (2-4C)alkynyl (optionally substituted with 1 group selected from R⁷), and HET-2;
R⁵ is independently at each occurrence selected from hydrogen, (1-4C)alkyl and (3-6C)cycloalkyl;
or R⁴ and R⁵ together with the nitrogen atom to which they are attached may form a heterocyclyl ring system as defined by HET-3;
R⁴′ and R^{5′} are independently selected from hydrogen and (1-4C)alkyl; or
R^{4′} and R^{5′} together with the nitrogen atom to which they are attached may form a 4- to 6-membered saturated ring;
R⁶ is selected from (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
HET-3 is an N-linked, 4- to 7-membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸;
R⁸ is selected from —OR⁵, (1-4C)alkyl, (2-4C)alkenyl, (2-4C)alkynyl, trifluoromethyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;
R⁹ is selected from (1-4C)alkyl, halo, cyano, hydroxy(1-4C)alkyl, dihydroxy(2-4C)alkyl, (1-4C)alkoxy(1-4C)alkyl, di(1-4C)alkoxy(2-4C)alkyl, (1-4C)alkylS(O)p(1-4C)alkyl, amino(1-4C)alkyl, (1-4C)alkylamino(1-4C)alkyl, di(1-4C)alkylamino(1-4C)alkyl, (1-4C)alkylcarbonylamino, (1-4C)alkylcarbonyl-N-[(1-4C)alkyl]amino, (1-4C)alkylaminocarbonyl, and di(1-4C)alkylaminocarbonyl;
R¹⁰ is selected from methoxy, methyl and halo;
p is (independently at each occurrence) 0, 1 or 2;
m is 0 or 1;
n is 0, 1 or 2;
or a salt or an in-vivo hydrolysable ester thereof;
with the proviso that:
i) neither R⁹ nor R¹⁰ is a substituent on X³;
ii) when R¹ is unsubstituted (1-6C)alkyl then L is —O—.

2. A compound of formula (I) as claimed in claim 1, or a salt or an in-vivo hydrolysable ester thereof, which is a compound of formula (IA)

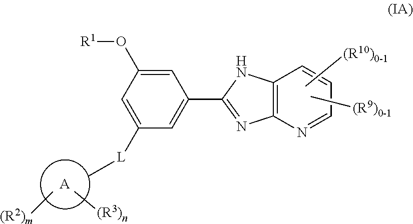

3. A compound of formula (I) as claimed in claim 1, or a salt or an in-vivo hydrolysable ester thereof, which is a compound of formula (IB)

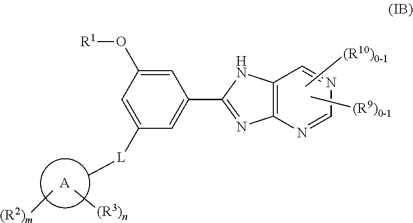

4. A compound of formula (I) as claimed in claim 1, or a salt or an in-vivo hydrolysable ester thereof, which is a compound of formula (IC)

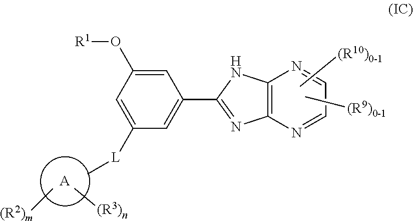

5. A compound of formula (I) as claimed in claim 1, or a salt or an in-vivo hydrolysable ester thereof wherein ring A is phenyl or HET-1 and HET-1 is fully unsaturated (aromatic) heterocyclic ring.

6. A compound of formula (I) as claimed in claim 5, or a salt or an in-vivo hydrolysable ester thereof wherein HET-1 is selected from pyridyl and pyrazinyl.

7. A compound of formula (I) as claimed in claim 1, or a salt or an in-vivo hydrolysable ester thereof wherein R¹ is selected from (1-4C)alkyl (optionally substituted by hydroxy or (1-4C)alkoxy) and HET-1 wherein HET-1 is a saturated 5- or 6-membered heterocyclic ring.

8. A compound of formula (I) as claimed in claim 1, or a salt or an in-vivo hydrolysable ester thereof wherein:
Ring A is selected from phenyl, pyridyl and pyrazinyl;
L is —O— or —CH₂O—;
R¹ is selected from hydroxyisopropyl, methoxyisopropyl, isopropyl, tert-butoxyisopropyl and tetrahydrofuran-2-yl;
R² is selected from methylsulfinyl, methylsulfonyl, ethylsulfonyl, prop-1-yn-3-ylaminocarbonyl, methylaminocarbonyl, dimethylaminocarbonyl, N-(cyanomethyl)-N-methylaminocarbonyl, cyclobutylaminocarbonyl, 1-(dimethylamino)ethylaminocarbonyl, methoxyethylaminocarbonyl, N-(hydroxyethyl)-N-methylaminocarbonyl, N-(N-methylpiperidin-4-yl)-N-(methyl)aminocarbonyl, aminocarbonylethylaminocarbonyl, 1-(dimethylamino)prop-2-ylaminocarbonyl, N-(hydroxyethyl)-N-(cyclobutyl)aminocarbonyl, pyrrolidinopropylamino, (2-hydroxytetrahydrothien-3-yl)aminocarbonyl, [4-(hydroxymethyptetrahydropyran-4-yl]aminocarbonyl, azetidinylcarbonyl, pyrrolidinylcarbonyl, (4-hydroxypiperidin-1-yl)carbonyl, (trifluoromethylpyrrolidinyl)carbonyl, N-methylpiperazinocarbonyl, 4-(methylaminocarbonyl)piperidin-1-ylcarbonyl, 4-(hydroxyethyl)piperazin-1-ylcarbonyl, 4-(isopropyl)piperazin-1-ylcarbonyl, (4-allyl)piperazin-1-ylcarbonyl, 1,1-dioxotetrahydrothien-3-ylaminocarbonyl, 1-(thien-2-yl)ethylaminocarbonyl, oxadiazolyl,
R³ is selected from fluoro, chloro, cyano, methoxy and carboxy;
R⁹ is selected from halo and cyano;
R¹⁰ is methoxy or halo;
m is 0 or 1;
n is 0 or 1.

9. A compound of formula (I) as claimed in claim 1, or a salt or an in-vivo hydrolysable ester thereof wherein:
Ring A is selected from phenyl, pyridyl and pyrazinyl;
L is —O— or —CH₂O—;
R¹ is selected from hydroxyisopropyl, methoxyisopropyl, isopropyl, tert-butoxyisopropyl and tetrahydrofuran-2-yl;
R² is selected from methylsulfinyl, methylsulfonyl, ethylsulfonyl, prop-1-yn-3-ylaminocarbonyl, methylaminocarbonyl, dimethylaminocarbonyl, N-(cyanomethyl)-N-methylaminocarbonyl, cyclobutylaminocarbonyl, 1-(dimethylamino)ethylaminocarbonyl, methoxyethylaminocarbonyl, N-(hydroxyethyl)-N-methylaminocarbonyl, N-(N-methylpiperidin-4-yl)-N-(methyl)aminocarbonyl, aminocarbonylethylaminocarbonyl, 1-(dimethylamino)prop-2-ylaminocarbonyl, N-(hydroxyethyl)-N-(cyclobutyl)aminocarbonyl, pyrrolidinopropylamino, (2-hydroxytetrahydrothien-3-yl)aminocarbonyl, [4-(hydroxymethyptetrahydropyran-4-yl]aminocarbonyl, azetidinylcarbonyl, pyrrolidinylcarbonyl, (4-hydroxypiperidin-1-yl)carbonyl, (trifluoromethylpyrrolidinyl)carbonyl, N-methylpiperazinocarbonyl, 4-(methylaminocarbonyl)piperidin-1-ylcarbonyl, 4-(hydroxyethyl)piperazin-1-ylcarbonyl, 4-(isopropyl)piperazin-1-ylcarbonyl, (4-allyl)piperazin-1-ylcarbonyl, 1,1-dioxotetrahydrothien-3-ylaminocarbonyl, 1-(thien-2-yl)ethylaminocarbonyl, oxadiazolyl,
R³ is selected from fluoro, chloro, cyano, methoxy and carboxy;
R⁹ is fluoro;
R¹⁰ is absent;
m is 0 or 1;
n is 0 or 1.

10. A compound of formula (I) as claimed in claim 1, or a salt or an in-vivo hydrolysable ester thereof which is a compound selected from:
2-{3-(benzyloxy)-5-[(1S)-2-methoxy-1-methylethoxy]phenyl}-3H-imidazo[4,5-b]pyridine;
8-{3-(benzyloxy)-5-[(1S)-2-methoxy-1-methylethoxy]phenyl}-9H-purine;
6-chloro-2-{3-[(1S)-2-methoxy-1-methylethoxy]-5-[4-(methylsulfonyl)phenoxy]phenyl}-3H-imidazo[4,5-b]pyridine;
8-{3-[(1S)-2-methoxy-1-methylethoxy]-5-[4-(methylsulfonyl)phenoxy]phenyl}-9H-purine;
2-{3-isopropoxy-5-[(1S)-1-methyl-2-phenylethoxy]phenyl}-3H-imidazo[4,5-b]pyridine;
6-bromo-2-{3-[(1S)-2-methoxy-1-methylethoxy]-5-[4-(methylsulfonyl)phenoxy]phenyl}-3H-imidazo[4,5-b]pyridine;
6-bromo-2-{3-[(1S)-2-methoxy-1-methylethoxy]-5-[4-(methylsulfonyl)phenoxy]phenyl}-3H-imidazo[4,5-b]pyridine;
2-{3-(benzyloxy)-5-[(1S)-2-methoxy-1-methylethoxy]phenyl}-5-bromo-1H-imidazo[4,5-b]pyrazine;
2-{3-[(1S)-2-methoxy-1-methylethoxy]-5-[4-(methylsulfonyl)phenoxy]phenyl}-3H-imidazo[4,5-b]pyridine;
2-{3-[4-(azetidin-1-ylcarbonyl)-2-fluorophenoxy]-5-[(1S)-2-methoxy-1-methylethoxy]phenyl}-3H-imidazo[4,5-b]pyridine;
3-chloro-4-{3-(3H-imidazo[4,5-b]pyridin-2-yl)-5-[(1S)-2-methoxy-1-methylethoxy]phenoxy}-N,N-dimethylbenzamide;
2-{3-[2-fluoro-4-(pyrrolidin-1-ylcarbonyl)phenoxy]-5-[(1S)-2-methoxy-1-methylethoxy]phenyl}-3H-imidazo[4,5-b]pyridine;
2-{3-[2-chloro-4-(pyrrolidin-1-ylcarbonyl)phenoxy]-5-[(1S)-2-methoxy-1-methylethoxy]phenyl}-3H-imidazo[4,5-b]pyridine;
2-{3-[2-fluoro-4-(methylsulfonyl)phenoxy]-5-[(1S)-2-methoxy-1-methylethoxy]phenyl}-3H-imidazo[4,5-b]pyridine;
3-fluoro-4-{3-(3H-imidazo[4,5-b]pyridin-2-yl)-5-[(1S)-2-methoxy-1-methylethoxy]phenoxy}-N,N-dimethylbenzamide;
4-{3-(3H-imidazo[4,5-b]pyridin-2-yl)-5-[(1S)-2-methoxy-1-methylethoxy]phenoxy}benzonitrile;
2-{3-(3H-imidazo[4,5-b]pyridin-2-yl)-5-[(1S)-2-methoxy-1-methylethoxy]phenoxy}benzonitrile;
2-{3-isopropoxy-5-[4-(methylsulfonyl)phenoxy]phenyl}-3H-imidazo[4,5-b]pyridine;
3-{3-(3H-imidazo[4,5-b]pyridin-2-yl)-5-[(1S)-2-methoxy-1-methylethoxy]phenoxy}-N,N-dimethylbenzamide;
2-{3-[(1S)-2-methoxy-1-methylethoxy]-5-[3-(methylsulfinyl)phenoxy]phenyl}-3H-imidazo[4,5-b]pyridine;
4-({3-(3H-imidazo[4,5-b]pyridin-2-yl)-5-[(1S)-2-methoxy-1-methylethoxy]phenoxy}methyl)benzonitrile;
2-({3-(3H-imidazo[4,5-b]pyridin-2-yl)-5-[(1S)-2-methoxy-1-methylethoxy]phenoxy}methyl)benzonitrile;
2-{3-[(3-methoxybenzyl)oxy]-5-[(1S)-2-methoxy-1-methylethoxy]phenyl}-3H-imidazo[4,5-b]pyridine;
2-{3-[(2-fluorobenzyl)oxy]-5-[(1S)-2-methoxy-1-methylethoxy]phenyl}-3H-imidazo[4,5-b]pyridine;
2-(3-[(1S)-2-methoxy-1-methylethoxy]-5-{[4-(methylsulfonyl)benzyl]oxy}phenyl)-3H-imidazo[4,5-b]pyridine;
4-({3-(3H-imidazo[4,5-b]pyridin-2-yl)-5-[(1S)-2-methoxy-1-methylethoxy]phenoxy}methyl)-N,N-dimethylbenzamide;
(2S)-2-{3-(3H-imidazo[4,5-b]pyridin-2-yl)-5-[4-(methylsulfonyl)phenoxy]phenoxy}propan-1-ol; and
(2S)-2-{3-(6-bromo-3H-imidazo[4,5-b]pyridin-2-yl)-5-[4-(methylsulfonyl)phenoxy]phenoxy}propan-1-ol;
2-{3-[(1S)-2-methoxy-1-methylethoxy]-5-[(1S)-1-methyl-2-phenylethoxy]phenyl}-3H-imidazo[4,5-b]pyridine;
2-{3-[(1S)-2-methoxy-1-methylethoxy]-5-[4-(methylsulfonyl)phenoxy]phenyl}-5-methyl-3H-imidazo[4,5-b]pyridine;
2-{3-[(1S)-2-methoxy-1-methylethoxy]-5-[4-(methylsulfonyl)phenoxy]phenyl}-6-methyl-3H-imidazo[4,5-b]pyridine;
2-{3-[(1S)-2-tert-butoxy-1-methylethoxy]-5-[4-(methylsulfonyl)phenoxy]phenyl}-5-methyl-3H-imidazo[4,5-b]pyridine;
2-{3-[(1S)-2-tert-butoxy-1-methylethoxy]-5-[4-(methylsulfonyl)phenoxy]phenyl}-6-methyl-3H-imidazo[4,5-b]pyridine;
2-{3-(benzyloxy)-5-[(1S)-2-methoxy-1-methylethoxy]phenyl}-5-methyl-3H-imidazo[4,5-b]pyridine;
(2S)-2-[3-[4-(azetidin-1-ylcarbonyl)-2-fluorophenoxy]-5-(3H-imidazo[4,5-b]pyridin-2-yl)phenoxy]propan-1-ol;
(2S)-2-[3-[4-(azetidin-1-ylcarbonyl)-2-fluorophenoxy]-5-(6-chloro-3H-imidazo[4,5-b]pyridin-2-yl)phenoxy]propan-1-ol;
8-{3-{[2-(azetidin-1-ylcarbonyl)pyrimidin-5-yl]oxy}-5-[(1S)-2-methoxy-1-methylethoxy]phenyl}-9H-purine;
5-{3-(3H-imidazo[4,5-b]pyridin-2-yl)-5-[(1S)-2-methoxy-1-methylethoxy]phenoxy}-N,N-dimethylpyrimidine-2-carboxamide;
5-[3-[(1S)-2-methoxy-1-methylethoxy]-5-(9H-purin-8-yl)phenoxy]-N,N-dimethylpyrimidine-2-carboxamide;
6-chloro-2-{3-[2-fluoro-4-(methylsulfonyl)phenoxy]-5-[(1S)-2-methoxy-1-methylethoxy]phenyl}-3H-imidazo[4,5-b]pyridine;
6-fluoro-2-{3-[(1S)-2-methoxy-1-methylethoxy]-5-[4-(methylsulfonyl)phenoxy]phenyl}-3H-imidazo[4,5-b]pyridine;
2-{3-{[5-(azetidin-1-ylcarbonyl)pyrazin-2-yl]oxy}-5-[(3S)-tetrahydrofuran-3-yloxy]phenyl}-1H-imidazo[4,5-b]pyridine;
8-{3-{[5-(azetidin-1-ylcarbonyl)pyrazin-2-yl]oxy}-5-[(3S)-tetrahydrofuran-3-yloxy]phenyl}-9H-purine;
8-{3-{[6-(azetidin-1-ylcarbonyl)pyridin-3-yl]oxy}-5-[(3S)-tetrahydrofuran-3-yloxy]phenyl}-9H-purine;
(2S)-2-{3-(6-chloro-3H-imidazo[4,5-b]pyridin-2-yl)-5-[4-(methylsulfonyl)phenoxy]phenoxy}propan-1-ol;
(2S)-2-{3-(5-methyl-3H-imidazo[4,5-b]pyridin-2-yl)-5-[4-(methylsulfonyl)phenoxy]phenoxy}propan-1-ol;
(2S)-2-(3-(3H-imidazo[4,5-b]pyridin-2-yl)-5-{4-[(4-methylpiperazin-1-yl)carbonyl]phenoxy}phenoxy)propan-1-ol;
(2S)-2-{3-(6-chloro-3H-imidazo[4,5-b]pyridin-2-yl)-5-[2-fluoro-4-(methylsulfonyl)phenoxy]phenoxy}propan-1-ol;
(2S)-2-{3-(6-fluoro-3H-imidazo[4,5-b]pyridin-2-yl)-5-[4-(methylsulfonyl)phenoxy]phenoxy}propan-1-ol;
5-[3-[(1S)-2-hydroxy-1-methylethoxy]-5-(9H-purin-8-yl)phenoxy]-N,N-dimethylpyrimidine-2-carboxamide;
5-[3-[(1S)-2-hydroxy-1-methylethoxy]-5-(9H-purin-8-yl)phenoxy]-N,N-dimethylpyrazine-2-carboxamide;
2-{3-[4-(ethylsulfonyl)-2-fluorophenoxy]-5-[(1S)-2-methoxy-1-methylethoxy]phenyl}-3H-imidazo[4,5-b]pyridine;
2-{3-[4-(azetidin-1-ylcarbonyl)-2-fluorophenoxy]-5-isopropoxyphenyl}-3H-imidazo[4,5-b]pyridine;
2-{3-[2-fluoro-4-(pyrrolidin-1-ylcarbonyl)phenoxy]-5-isopropoxyphenyl}-3H-imidazo[4,5-b]pyridine;
3-chloro-4-[3-(3H-imidazo[4,5-b]pyridin-2-yl)-5-isopropoxyphenoxy]-N,N-dimethylbenzamide;
5-chloro-6-{3-(3H-imidazo[4,5-b]pyridin-2-yl)-5-[(1S)-2-methoxy-1-methylethoxy]phenoxy}-N,N-dimethylnicotinamide;
5-methoxy-2-{3-[(1S)-2-methoxy-1-methylethoxy]-5-[4-(methylsulfonyl)phenoxy]phenyl}-1H-imidazo[4,5-b]pyrazine;
2-{3-[(1S)-2-methoxy-1-methylethoxy]-5-[4-(1,2,4-oxadiazol-3-yl)phenoxy]phenyl}-3H-imidazo[4,5-b]pyridine;
2-{3-[(1S)-2-methoxy-1-methylethoxy]-5-[4-(methylsulfonyl)phenoxy]phenyl}-3H-imidazo[4,5-b]pyridine-6-carbonitrile;
N-[2-(dimethylamino)ethyl]-6-{3-(3H-imidazo[4,5-b]pyridin-2-yl)-5-[(1S)-2-methoxy-1-methylethoxy]phenoxy}nicotinamide;
4-{3-(3H-imidazo[4,5-b]pyridin-2-yl)-5-[(1S)-2-methoxy-1-methylethoxy]phenoxy}benzoic acid;
2-{3-[(1S)-2-methoxy-1-methylethoxy]-5-[4-(pyrrolidin-1-ylcarbonyl)phenoxy]phenyl}-3H-imidazo[4,5-b]pyridine;
2-[3-[(1S)-2-methoxy-1-methylethoxy]-5-(4-{[2-(trifluoromethyl)pyrrolidin-1-yl]carbonyl}phenoxy)phenyl]-3H-imidazo[4,5-b]pyridine;
4-{3-(3H-imidazo[4,5-b]pyridin-2-yl)-5-[(1S)-2-methoxy-1-methylethoxy]phenoxy}-N-[1-(2-thienyl)ethyl]benzamide;
N-(2-hydroxyethyl)-4-{3-(3H-imidazo[4,5-b]pyridin-2-yl)-5-[(1S)-2-methoxy-1-methylethoxy]phenoxy}-N-methylbenzamide;
4-{3-(3H-imidazo[4,5-b]pyridin-2-yl)-5-[(1S)-2-methoxy-1-methylethoxy]phenoxy}-N-methyl-N-(1-methylpiperidin-4-yl)benzamide;
N-(3-amino-3-oxopropyl)-4-{3-(3H-imidazo[4,5-b]pyridin-2-yl)-5-[(1S)-2-methoxy-1-methylethoxy]phenoxy}benzamide;
N-[4-(hydroxymethyl)tetrahydro-2H-pyran-4-yl]-4-{3-(3H-imidazo[4,5-b]pyridin-2-yl)-5-[(1S)-2-methoxy-1-methylethoxy]phenoxy}benzamide;
N-cyclobutyl-4-{3-(3H-imidazo[4,5-b]pyridin-2-yl)-5-[(1S)-2-methoxy-1-methylethoxy]phenoxy}benzamide;
4-{3-(3H-imidazo[4,5-b]pyridin-2-yl)-5-[(1S)-2-methoxy-1-methylethoxy]phenoxy}-N-prop-2-yn-1-ylbenzamide;
N-(1,1-dioxidotetrahydro-3-thienyl)-4-{3-(3H-imidazo[4,5-b]pyridin-2-yl)-5-[(1S)-2-methoxy-1-methylethoxy]phenoxy}benzamide;
1-(4-{3-(3H-imidazo[4,5-b]pyridin-2-yl)-5-[(1S)-2-methoxy-1-methylethoxy]phenoxy}benzoyl)-N-methylpiperidine-4-carboxamide;
2-[4-(4-{3-(3H-imidazo[4,5-b]pyridin-2-yl)-5-[(1S)-2-methoxy-1-methylethoxy]phenoxy}benzoyl)piperazin-1-yl]ethanol;
4-{3-(3H-imidazo[4,5-b]pyridin-2-yl)-5-[(1S)-2-methoxy-1-methylethoxy]phenoxy}-N-(2-methoxyethyl)benzamide;
1-(4-{3-(3H-imidazo[4,5-b]pyridin-2-yl)-5-[(1S)-2-methoxy-1-methylethoxy]phenoxy}benzoyl)piperidin-4-ol;
4-{3-(3H-imidazo[4,5-b]pyridin-2-yl)-5-[(1S)-2-methoxy-1-methylethoxy]phenoxy}-N-methylbenzamide;
2-{3-(4-[(4-isopropylpiperazin-1-yl)carbonyl]phenoxy}-5-[(1S)-2-methoxy-1-methylethoxy]phenyl}-3H-imidazo[4,5-b]pyridine;
N-(cyanomethyl)-4-{3-(3H-imidazo[4,5-b]pyridin-2-yl)-5-[(1S)-2-methoxy-1-methylethoxy]phenoxy}-N-methylbenzamide;
N-(4-hydroxytetrahydro-3-thienyl)-4-{3-(3H-imidazo[4,5-b]pyridin-2-yl)-5-[(1S)-2-methoxy-1-methylethoxy]phenoxy}benzamide;
N-cyclobutyl-N-(2-hydroxyethyl)-4-{3-(3H-imidazo[4,5-b]pyridin-2-yl)-5-[(1S)-2-methoxy-1-methylethoxy]phenoxy}benzamide;
2-{3-(4-[(4-allylpiperazin-1-yl)carbonyl]phenoxy}-5-[(1S)-2-methoxy-1-methylethoxy]phenyl)-3H-imidazo[4,5-b]pyridine;
4-{3-(3H-imidazo[4,5-b]pyridin-2-yl)-5-[(1S)-2-methoxy-1-methylethoxy]phenoxy}-N-(3-pyrrolidin-1-ylpropyl)benzamide;
N-[2-(dimethylamino)-1-methylethyl]-4-{3-(3H-imidazo[4,5-b]pyridin-2-yl)-5-[(1S)-2-methoxy-1-methylethoxy]phenoxy}benzamide;
4-{3-(3H-imidazo[4,5-b]pyridin-2-yl)-5-[(1S)-2-methoxy-1-methylethoxy]phenoxy}-N,N-dimethylbenzamide;
2-(3-[(1S)-2-methoxy-1-methylethoxy]-5-{4-[(4-methylpiperazin-1-yl)carbonyl]phenoxy}phenyl)-3H-imidazo[4,5-b]pyridine; and
2-{3-[(1S)-2-methoxy-1-methylethoxy]-5-[4-(methylsulfonyl)phenoxy]phenyl}-1H-imidazo[4,5-b]pyrazine;
2-{3-{[6-(azetidin-1-ylcarbonyl)pyridin-3-yl]oxy}-5-[(3S)-tetrahydrofuran-3-yloxy]phenyl}-6-fluoro-3H-imidazo[4,5-b]pyridine; and
:N,N-dimethyl-5-({3-(9H-purin-8-yl)-5-[(3S)-tetrahydrofuran-3-yloxy]phenyl}oxy)pyrazine-2-carboxamide.

11. A pharmaceutical composition comprising a compound according to claim 1, or a pharmaceutically-acceptable salt or an in-vivo hydrolysable ester thereof, together with a pharmaceutically acceptable diluent or carrier.

12.-16. (canceled)

17. A method of treating GLK mediated diseases by administering an effective amount of a compound of Formula (I) as claimed in claim 1 or a pharmaceutically-acceptable salt or an in-vivo hydrolysable ester thereof, to a mammal in need of such treatment.

18. The method of claim 17 wherein the GLK mediated disease is type 2 diabetes.

19. A process for preparing a compound of Formula (I), which comprises a process a) to 0 (wherein the variables are as defined in claim 1 unless otherwise defined):
(a) reacting a compound of Formula (III) with a compound of Formula (IV), 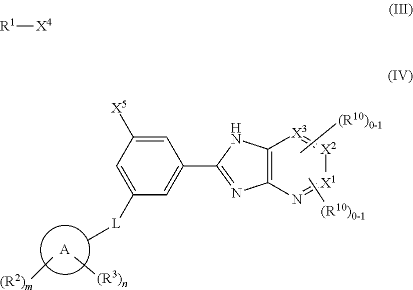
wherein X⁴ is a leaving group or an organometallic reagent and X⁵ is a hydroxyl group, or X⁴ is a hydroxyl group and X⁵ is a leaving group or an organometallic reagent, and wherein R¹ is as defined for a compound of formula (I), or is a protected version thereof;
or
(b) reacting a compound of Formula (V) with a compound of Formula (VI) 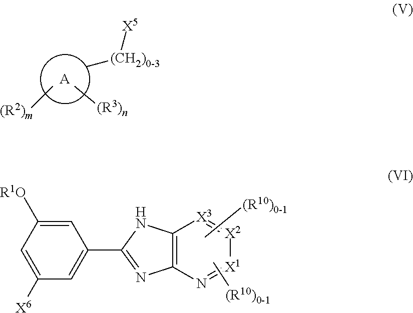
wherein X⁶ is a leaving group or an organometallic reagent and X⁷ is a hydroxyl group, or X⁶ is a hydroxyl group and X⁷ is a leaving group or an organometallic reagent, and wherein R¹ is as defined for a compound of formula (I), or a protected version thereof;
or
(c) reacting a compound of Formula (VII) with a compound of Formula (VIII), 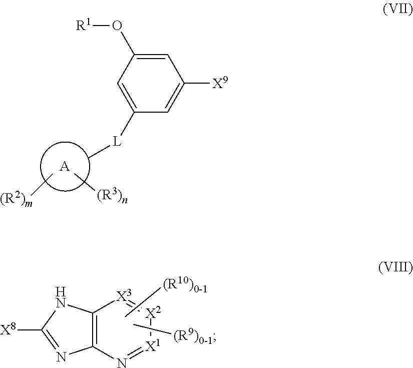
wherein X⁸ is a leaving group and X⁹ is an organometallic agent, or X⁸ is a leaving group and X⁹ is an organometallic agent; and wherein R¹ is as defined for a compound of formula (I) or a protected version thereof;
or
(d) reacting a compound of formula (IX) with a compound of formula (X) and cyclisation in a one or two step reaction; 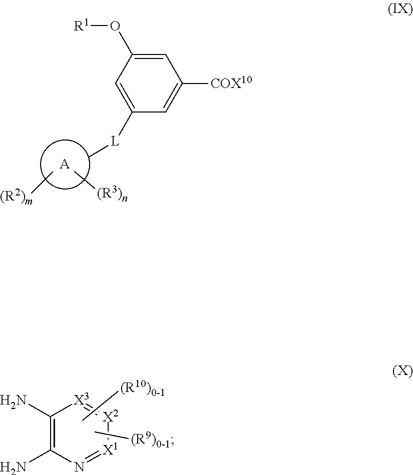
wherein X¹⁰ is hydrogen, a hydroxyl group, a halogen, or other leaving group, and wherein R¹ is as defined for a compound of formula (I) or a protected version thereof;
or
e) reaction a compound of formula (XI) with a compound of formula (XII) and cyclisation in a one step reaction, 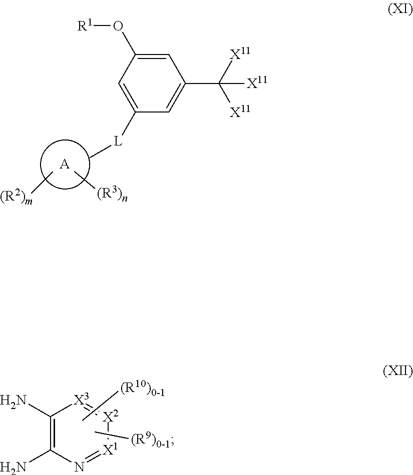
wherein each X¹¹ is a leaving group, and wherein R¹ is as defined for a compound of formula (I) or a protected version thereof;
or
f) reacting a compound of formula (XIII) with a compound of formula (XIV) and cyclisation in a one or two step reaction, 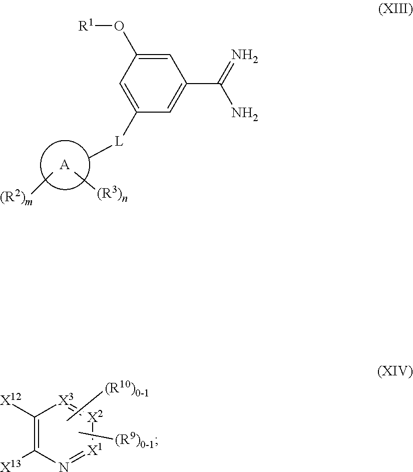
wherein X¹² and X¹³ are independently halogen or other leaving group, and wherein R¹ is as defined for a compound of formula (I) or a protected version thereof;
and optionally thereafter:
i) converting a compound of Formula (I) into another compound of Formula (I);
ii) removing any protecting groups; and/or
iii) forming a salt or an in-vivo hydrolysable ester thereof.
